SEQUENCE LISTING (473 sequences)

SEQ ID NO: 1 - DNA, 20 bp, synthetic construct
  misc_feature 1..20: note = Synthetic
  source 1..20: mol_type = other DNA
tcctcccacttggcgtccag

SEQ ID NO: 2 - DNA, 20 bp, synthetic construct
  misc_feature 1..20: note = Synthetic
  source 1..20: mol_type = other DNA
agaagcagagcgggaggccc

SEQ ID NO: 3 - DNA, 20 bp, synthetic construct
  misc_feature 1..20: note = Synthetic
  source 1..20: mol_type = other DNA
ttccccacagctgtggtctg

SEQ ID NO: 4 - DNA, 20 bp, synthetic construct
  misc_feature 1..20: note = Synthetic
  source 1..20: mol_type = other DNA
gtggaggcttcctctactcg

SEQ ID NO: 5 - DNA, 20 bp, synthetic construct
  misc_feature 1..20: note = Synthetic
  source 1..20: mol_type = other DNA
aggaggaaagagtggaggtg

SEQ ID NO: 6 - DNA, 20 bp, synthetic construct
  misc_feature 1..20: note = Synthetic
  source 1..20: mol_type = other DNA
ggtgctgtctggcggttgag

SEQ ID NO: 7 - DNA, 20 bp, synthetic construct
  misc_feature 1..20: note = Synthetic
  source 1..20: mol_type = other DNA
ttcaaagctagcctctggct

SEQ ID NO: 8 - DNA, 20 bp, synthetic construct
  misc_feature 1..20: note = Synthetic
  source 1..20: mol_type = other DNA
ttagggccccccactgatga

SEQ ID NO: 9 - DNA, 20 bp, synthetic construct
  misc_feature 1..20: note = Synthetic
  source 1..20: mol_type = other DNA
ggcaggttcggtgtcacatg

SEQ ID NO: 10 - DNA, 20 bp, synthetic construct
  misc_feature 1..20: note = Synthetic
  source 1..20: mol_type = other DNA
agaggctgatgcattgttga

SEQ ID NO: 11 - DNA, 20 bp, synthetic construct
  misc_feature 1..20: note = Synthetic
  source 1..20: mol_type = other DNA
tggttgggcccccaggccag

SEQ ID NO: 12 - DNA, 20 bp, synthetic construct
  misc_feature 1..20: note = Synthetic
  source 1..20: mol_type = other DNA
gcagatgaccaggtagtggt

SEQ ID NO: 13 - DNA, 20 bp, synthetic construct
  misc_feature 1..20: note = Synthetic
  source 1..20: mol_type = other DNA
ggagagggcagatgaccagg

SEQ ID NO: 14 - DNA, 20 bp, synthetic construct
  misc_feature 1..20: note = Synthetic
  source 1..20: mol_type = other DNA
ggtgaaggagccagagttgg

SEQ ID NO: 15 - DNA, 20 bp, synthetic construct
  misc_feature 1..20: note = Synthetic
  source 1..20: mol_type = other DNA
gagacagagaggcttgttgg

SEQ ID NO: 16 - DNA, 20 bp, synthetic construct
  misc_feature 1..20: note = Synthetic
  source 1..20: mol_type = other DNA
attggagacagagaggcttg

SEQ ID NO: 17 - DNA, 20 bp, synthetic construct
  misc_feature 1..20: note = Synthetic
  source 1..20: mol_type = other DNA
ctgattggagacagagaggc

SEQ ID NO: 18 - DNA, 20 bp, synthetic construct
  misc_feature 1..20: note = Synthetic
  source 1..20: mol_type = other DNA
ggctgattggagacagagag

SEQ ID NO: 19 - DNA, 20 bp, synthetic construct
  misc_feature 1..20: note = Synthetic
  source 1..20: mol_type = other DNA
agagaaggctgagtatactt

SEQ ID NO: 20 - DNA, 20 bp, synthetic construct
  misc_feature 1..20: note = Synthetic
  source 1..20: mol_type = other DNA
accctggctccacacagcct

SEQ ID NO: 21 - DNA, 20 bp, synthetic construct
  misc_feature 1..20: note = Synthetic
  source 1..20: mol_type = other DNA
gcattgctgttggctaagag

SEQ ID NO: 22 - DNA, 20 bp, synthetic construct
  misc_feature 1..20: note = Synthetic
  source 1..20: mol_type = other DNA
ctgttgctgttgctggtggt

SEQ ID NO: 23 - DNA, 20 bp, synthetic construct
  misc_feature 1..20: note = Synthetic
  source 1..20: mol_type = other DNA
ggcccagagtttccgtgatg

SEQ ID NO: 24 - DNA, 20 bp, synthetic construct
  misc_feature 1..20: note = Synthetic
  source 1..20: mol_type = other DNA
ggagctaccgccctccagtg

SEQ ID NO: 25 - DNA, 20 bp, synthetic construct
  misc_feature 1..20: note = Synthetic
  source 1..20: mol_type = other DNA
catggcgtaagggtgtgcgt

SEQ ID NO: 26 - DNA, 20 bp, synthetic construct
  misc_feature 1..20: note = Synthetic
  source 1..20: mol_type = other DNA
aggacacctggctgtgaggt

SEQ ID NO: 27 - DNA, 20 bp, synthetic construct
  misc_feature 1..20: note = Synthetic
  source 1..20: mol_type = other DNA
gcctgcttggctgtaggaca

SEQ ID NO: 28 - DNA, 20 bp, synthetic construct
  misc_feature 1..20: note = Synthetic
  source 1..20: mol_type = other DNA
gaagagactggagggccatt

SEQ ID NO: 29 - DNA, 20 bp, synthetic construct
  misc_feature 1..20: note = Synthetic
  source 1..20: mol_type = other DNA
ccacggtggcaatgaccgtg

SEQ ID NO: 30 - DNA, 20 bp, synthetic construct
  misc_feature 1..20: note = Synthetic
  source 1..20: mol_type = other DNA
gaggccgttttgtagcctgc

SEQ ID NO: 31 - DNA, 20 bp, synthetic construct
  misc_feature 1..20: note = Synthetic
  source 1..20: mol_type = other DNA
ggctctctttccgtacggtg

SEQ ID NO: 32 - DNA, 20 bp, synthetic construct
  misc_feature 1..20: note = Synthetic
  source 1..20: mol_type = other DNA
gcgacccgggtttgtatccg

SEQ ID NO: 33 - DNA, 20 bp, synthetic construct
  misc_feature 1..20: note = Synthetic
  source 1..20: mol_type = other DNA
cgaggttcctcgatagcccg

SEQ ID NO: 34 - DNA, 20 bp, synthetic construct
  misc_feature 1..20: note = Synthetic
  source 1..20: mol_type = other DNA
tcccacggtgggcgagcccg

SEQ ID NO: 35 - DNA, 20 bp, synthetic construct
  misc_feature 1..20: note = Synthetic
  source 1..20: mol_type = other DNA
ggtggtggcagcgatggcag

SEQ ID NO: 36 - DNA, 20 bp, synthetic construct
  misc_feature 1..20: note = Synthetic
  source 1..20: mol_type = other DNA
cagcttggagccctccagtg

SEQ ID NO: 37 - DNA, 20 bp, synthetic construct
  misc_feature 1..20: note = Synthetic
  source 1..20: mol_type = other DNA
ctgctcggcctcgcgccgca

SEQ ID NO: 38 - DNA, 20 bp, synthetic construct
  misc_feature 1..20: note = Synthetic
  source 1..20: mol_type = other DNA
tcttcgcgcgcgcgctgctc

SEQ ID NO: 39 - DNA, 20 bp, synthetic construct
  misc_feature 1..20: note = Synthetic
  source 1..20: mol_type = other DNA
tctttctcgcgttcccgctc

SEQ ID NO: 40 - DNA, 20 bp, synthetic construct
  misc_feature 1..20: note = Synthetic
  source 1..20: mol_type = other DNA
tcaagctcgcgctccttctc

SEQ ID NO: 41 - DNA, 20 bp, synthetic construct
  misc_feature 1..20: note = Synthetic
  source 1..20: mol_type = other DNA
acgctgcgttcaagctcgcg

SEQ ID NO: 42 - DNA, 20 bp, synthetic construct
  misc_feature 1..20: note = Synthetic
  source 1..20: mol_type = other DNA
gcattccaccggagcacggc

SEQ ID NO: 43 - DNA, 20 bp, synthetic construct
  misc_feature 1..20: note = Synthetic
  source 1..20: mol_type = other DNA
cgcactgcccggttcaaatg

SEQ ID NO: 44 - DNA, 20 bp, synthetic construct
  misc_feature 1..20: note = Synthetic
  source 1..20: mol_type = other DNA
gcttcccgttccctctcccg

SEQ ID NO: 45 - DNA, 20 bp, synthetic construct
  misc_feature 1..20: note = Synthetic
  source 1..20: mol_type = other DNA
cacggaggtctcgttcacgg

SEQ ID NO: 46 - DNA, 20 bp, synthetic construct
  misc_feature 1..20: note = Synthetic
  source 1..20: mol_type = other DNA
gaaagggatccaagcccggc

SEQ ID NO: 47 - DNA, 20 bp, synthetic construct
  misc_feature 1..20: note = Synthetic
  source 1..20: mol_type = other DNA
gccaggctgcagagccaggc

SEQ ID NO: 48 - DNA, 20 bp, synthetic construct
  misc_feature 1..20: note = Synthetic
  source 1..20: mol_type = other DNA
ctagacgttctcgctccagg

SEQ ID NO: 49 - DNA, 20 bp, synthetic construct
  misc_feature 1..20: note = Synthetic
  source 1..20: mol_type = other DNA
gctggcccagctgccagcgc

SEQ ID NO: 50 - DNA, 20 bp, synthetic construct
  misc_feature 1..20: note = Synthetic
  source 1..20: mol_type = other DNA
gtgcaggtgcgagtggatgt

SEQ ID NO: 51 - DNA, 20 bp, synthetic construct
  misc_feature 1..20: note = Synthetic
  source 1..20: mol_type = other DNA
agaacttcgttctcgtgcag

SEQ ID NO: 52 - DNA, 20 bp, synthetic construct
  misc_feature 1..20: note = Synthetic
  source 1..20: mol_type = other DNA
gaggccggcaggtcccggta

SEQ ID NO: 53 - DNA, 20 bp, synthetic construct
  misc_feature 1..20: note = Synthetic
  source 1..20: mol_type = other DNA
gcagaaagggaggccggcag

SEQ ID NO: 54 - DNA, 20 bp, synthetic construct
  misc_feature 1..20: note = Synthetic
  source 1..20: mol_type = other DNA
gtgcatggcctgcagctgat

SEQ ID NO: 55 - DNA, 20 bp, synthetic construct
  misc_feature 1..20: note = Synthetic
  source 1..20: mol_type = other DNA
catgcagccactgctgctgt

SEQ ID NO: 56 - DNA, 20 bp, synthetic construct
  misc_feature 1..20: note = Synthetic
  source 1..20: mol_type = other DNA
gcagcggcacactgtgcagc

SEQ ID NO: 57 - DNA, 20 bp, synthetic construct
  misc_feature 1..20: note = Synthetic
  source 1..20: mol_type = other DNA
ccctccctcccccactctct

SEQ ID NO: 58 - DNA, 20 bp, synthetic construct
  misc_feature 1..20: note = Synthetic
  source 1..20: mol_type = other DNA
gccacctccccacctctgca

SEQ ID NO: 59 - DNA, 20 bp, synthetic construct
  misc_feature 1..20: note = Synthetic
  source 1..20: mol_type = other DNA
gcaggggacagccttgaagt

SEQ ID NO: 60 - DNA, 20 bp, synthetic construct
  misc_feature 1..20: note = Synthetic
  source 1..20: mol_type = other DNA
ctcgggccttcttggctgtc

SEQ ID NO: 61 - DNA, 20 bp, synthetic construct
  misc_feature 1..20: note = Synthetic
  source 1..20: mol_type = other DNA
ctcactttcactctctgaga

SEQ ID NO: 62 - DNA, 20 bp, synthetic construct
  misc_feature 1..20: note = Synthetic
  source 1..20: mol_type = other DNA
ggtctcctcactttcactct

SEQ ID NO: 63 - DNA, 20 bp, synthetic construct
  misc_feature 1..20: note = Synthetic
  source 1..20: mol_type = other DNA
ggtcgctgctgccatcatca

SEQ ID NO: 64 - DNA, 20 bp, synthetic construct
  misc_feature 1..20: note = Synthetic
  source 1..20: mol_type = other DNA
tagggtcgctgctgccatca

SEQ ID NO: 65 - DNA, 20 bp, synthetic construct
  misc_feature 1..20: note = Synthetic
  source 1..20: mol_type = other DNA
gggacgtgcttcggttgtcc

SEQ ID NO: 66 - DNA, 20 bp, synthetic construct
  misc_feature 1..20: note = Synthetic
  source 1..20: mol_type = other DNA
ctgtcgaggggtgctgtctg

SEQ ID NO: 67 - DNA, 20 bp, synthetic construct
  misc_feature 1..20: note = Synthetic
  source 1..20: mol_type = other DNA
tggctgtcgaggggtgctgt

SEQ ID NO: 68 - DNA, 20 bp, synthetic construct
  misc_feature 1..20: note = Synthetic
  source 1..20: mol_type = other DNA
aagctagcctctggctgtcg

SEQ ID NO: 69 - DNA, 20 bp, synthetic construct
  misc_feature 1..20: note = Synthetic
  source 1..20: mol_type = other DNA
gttcaaagctagcctctggc

SEQ ID NO: 70 - DNA, 20 bp, synthetic construct
  misc_feature 1..20: note = Synthetic
  source 1..20: mol_type = other DNA
ggggttcaaagctagcctct

SEQ ID NO: 71 - DNA, 20 bp, synthetic construct
  misc_feature 1..20: note = Synthetic
  source 1..20: mol_type = other DNA
atatccagtgggtgtcacag

SEQ ID NO: 72 - DNA, 20 bp, synthetic construct
  misc_feature 1..20: note = Synthetic
  source 1..20: mol_type = other DNA
ggaggagcctggaacattcg

SEQ ID NO: 73 - DNA, 20 bp, synthetic construct
  misc_feature 1..20: note = Synthetic
  source 1..20: mol_type = other DNA
ggtctcagggcaggtggggg

SEQ ID NO: 74 - DNA, 20 bp, synthetic construct
  misc_feature 1..20: note = Synthetic
  source 1..20: mol_type = other DNA
aggggtctcagggcaggtgg

SEQ ID NO: 75 - DNA, 20 bp, synthetic construct
  misc_feature 1..20: note = Synthetic
  source 1..20: mol_type = other DNA
ttgaggggtctcagggcagg

SEQ ID NO: 76 - DNA, 20 bp, synthetic construct
  misc_feature 1..20: note = Synthetic
  source 1..20: mol_type = other DNA
gcattgttgaggggtctcag

SEQ ID NO: 77 - DNA, 20 bp, synthetic construct
  misc_feature 1..20: note = Synthetic
  source 1..20: mol_type = other DNA
atgcattgttgaggggtctc

SEQ ID NO: 78 - DNA, 20 bp, synthetic construct
  misc_feature 1..20: note = Synthetic
  source 1..20: mol_type = other DNA
gggagaggctgatgcattgt

SEQ ID NO: 79 - DNA, 20 bp, synthetic construct
  misc_feature 1..20: note = Synthetic
  source 1..20: mol_type = other DNA
caggccagggggagaggctg

SEQ ID NO: 80 - DNA, 20 bp, synthetic construct
  misc_feature 1..20: note = Synthetic
  source 1..20: mol_type = other DNA
ccccaggccagggggagagg

SEQ ID NO: 81 - DNA, 20 bp, synthetic construct
  misc_feature 1..20: note = Synthetic
  source 1..20: mol_type = other DNA
ggcccccaggccagggggag

SEQ ID NO: 82 - DNA, 20 bp, synthetic construct
  misc_feature 1..20: note = Synthetic
  source 1..20: mol_type = other DNA
ttgggcccccaggccagggg

SEQ ID NO: 83 - DNA, 20 bp, synthetic construct
  misc_feature 1..20: note = Synthetic
  source 1..20: mol_type = other DNA
tgaccaggtagtggttgggc

SEQ ID NO: 84 - DNA, 20 bp, synthetic construct
  misc_feature 1..20: note = Synthetic
  source 1..20: mol_type = other DNA
gagagggcagatgaccaggt

SEQ ID NO: 85 - DNA, 20 bp, synthetic construct
  misc_feature 1..20: note = Synthetic
  source 1..20: mol_type = other DNA
ggggagagggcagatgacca

SEQ ID NO: 86 - DNA, 20 bp, synthetic construct
  misc_feature 1..20: note = Synthetic
  source 1..20: mol_type = other DNA
ccatggcgtggggagagggc

SEQ ID NO: 87 - DNA, 20 bp, synthetic construct
  misc_feature 1..20: note = Synthetic
  source 1..20: mol_type = other DNA
gtcccatggcgtggggagag

SEQ ID NO: 88 - DNA, 20 bp, synthetic construct
  misc_feature 1..20: note = Synthetic
  source 1..20: mol_type = other DNA
ccctgtcccatggcgtgggg

SEQ ID NO: 89 - DNA, 20 bp, synthetic construct
  misc_feature 1..20: note = Synthetic
  source 1..20: mol_type = other DNA
ggcccttctctgggccagga

SEQ ID NO: 90 - DNA, 20 bp, synthetic construct
  misc_feature 1..20: note = Synthetic
  source 1..20: mol_type = other DNA
gaagaggaggctgctgcaga

SEQ ID NO: 91 - DNA, 20 bp, synthetic construct
  misc_feature 1..20: note = Synthetic
  source 1..20: mol_type = other DNA
tgggcaatgcctgggaagct

SEQ ID NO: 92 - DNA, 20 bp, synthetic construct
  misc_feature 1..20: note = Synthetic
  source 1..20: mol_type = other DNA
agtatacttggggggctgat

SEQ ID NO: 93 - DNA, 20 bp, synthetic construct
  misc_feature 1..20: note = Synthetic
  source 1..20: mol_type = other DNA
cagcctgggatgggagagaa

SEQ ID NO: 94 - DNA, 20 bp, synthetic construct
  misc_feature 1..20: note = Synthetic
  source 1..20: mol_type = other DNA
taagaggcggccatagggag

SEQ ID NO: 95 - DNA, 20 bp, synthetic construct
  misc_feature 1..20: note = Synthetic
  source 1..20: mol_type = other DNA
ctgttggctaagaggcggcc

SEQ ID NO: 96 - DNA, 20 bp, synthetic construct
  misc_feature 1..20: note = Synthetic
  source 1..20: mol_type = other DNA
ttgctgttggctaagaggcg

SEQ ID NO: 97 - DNA, 20 bp, synthetic construct
  misc_feature 1..20: note = Synthetic
  source 1..20: mol_type = other DNA
tgggcattgctgttggctaa

SEQ ID NO: 98 - DNA, 20 bp, synthetic construct
  misc_feature 1..20: note = Synthetic
  source 1..20: mol_type = other DNA
ggatgggcattgctgttggc

SEQ ID NO: 99 - DNA, 20 bp, synthetic construct
  misc_feature 1..20: note = Synthetic
  source 1..20: mol_type = other DNA
cctggatgggcattgctgtt

SEQ ID NO: 100 - DNA, 20 bp, synthetic construct
  misc_feature 1..20: note = Synthetic
  source 1..20: mol_type = other DNA
gggcctggatgggcattgct

SEQ ID NO: 101 - DNA, 20 bp, synthetic construct
  misc_feature 1..20: note = Synthetic
  source 1..20: mol_type = other DNA
aaggggcctggatgggcatt

SEQ ID NO: 102 - DNA, 20 bp, synthetic construct
  misc_feature 1..20: note = Synthetic
  source 1..20: mol_type = other DNA
gggaaggggcctggatgggc

SEQ ID NO: 103 - DNA, 20 bp, synthetic construct
  misc_feature 1..20: note = Synthetic
  source 1..20: mol_type = other DNA
gaaatgctccaggaggaggg

SEQ ID NO: 104 - DNA, 20 bp, synthetic construct
  misc_feature 1..20: note = Synthetic
  source 1..20: mol_type = other DNA
gcaggtgtgctggccctggt

SEQ ID NO: 105 - DNA, 20 bp, synthetic construct
  misc_feature 1..20: note = Synthetic
  source 1..20: mol_type = other DNA
ggcaggtgtgctggccctgg

SEQ ID NO: 106 - DNA, 20 bp, synthetic construct
  misc_feature 1..20: note = Synthetic
  source 1..20: mol_type = other DNA
gggggcaggtgtgctggccc

SEQ ID NO: 107 - DNA, 20 bp, synthetic construct
  misc_feature 1..20: note = Synthetic
  source 1..20: mol_type = other DNA
ggtgggggcaggtgtgctgg

SEQ ID NO: 108 - DNA, 20 bp, synthetic construct
  misc_feature 1..20: note = Synthetic
  source 1..20: mol_type = other DNA
tgaggtgggggcaggtgtgc

SEQ ID NO: 109 - DNA, 20 bp, synthetic construct
  misc_feature 1..20: note = Synthetic
  source 1..20: mol_type = other DNA
ctgtgaggtgggggcaggtg

SEQ ID NO: 110 - DNA, 20 bp, synthetic construct
  misc_feature 1..20: note = Synthetic
  source 1..20: mol_type = other DNA
tggctgtgaggtgggggcag

SEQ ID NO: 111 - DNA, 20 bp, synthetic construct
  misc_feature 1..20: note = Synthetic
  source 1..20: mol_type = other DNA
acctggctgtgaggtggggg

SEQ ID NO: 112 - DNA, 20 bp, synthetic construct
  misc_feature 1..20: note = Synthetic
  source 1..20: mol_type = other DNA
ttggctgtaggacacctggc

SEQ ID NO: 113 - DNA, 20 bp, synthetic construct
  misc_feature 1..20: note = Synthetic
  source 1..20: mol_type = other DNA
tgcttggctgtaggacacct

SEQ ID NO: 114 - DNA, 20 bp, synthetic construct
  misc_feature 1..20: note = Synthetic
  source 1..20: mol_type = other DNA
attggggcctgcttggctgt

SEQ ID NO: 115 - DNA, 20 bp, synthetic construct
  misc_feature 1..20: note = Synthetic
  source 1..20: mol_type = other DNA
ggcgccccttgagggccctg

SEQ ID NO: 116 - DNA, 20 bp, synthetic construct
  misc_feature 1..20: note = Synthetic
  source 1..20: mol_type = other DNA
tggcgaggaagccacggtgg

SEQ ID NO: 117 - DNA, 20 bp, synthetic construct
  misc_feature 1..20: note = Synthetic
  source 1..20: mol_type = other DNA
gctggcgaggaagccacggt

SEQ ID NO: 118 - DNA, 20 bp, synthetic construct
  misc_feature 1..20: note = Synthetic
  source 1..20: mol_type = other DNA
ggcccaggtggggaggccgt

SEQ ID NO: 119 - DNA, 20 bp, synthetic construct
  misc_feature 1..20: note = Synthetic
  source 1..20: mol_type = other DNA
gcccccggggacggggctct

SEQ ID NO: 120 - DNA, 20 bp, synthetic construct
  misc_feature 1..20: note = Synthetic
  source 1..20: mol_type = other DNA
agcccggtggggtccccgtt

SEQ ID NO: 121 - DNA, 20 bp, synthetic construct
  misc_feature 1..20: note = Synthetic
  source 1..20: mol_type = other DNA
agcccggcttgaaggtccct

SEQ ID NO: 122 - DNA, 20 bp, synthetic construct
  misc_feature 1..20: note = Synthetic
  source 1..20: mol_type = other DNA
gtggcaggggcccaggtccc

SEQ ID NO: 123 - DNA, 20 bp, synthetic construct
  misc_feature 1..20: note = Synthetic
  source 1..20: mol_type = other DNA
caggcctgagggccccgcag

SEQ ID NO: 124 - DNA, 20 bp, synthetic construct
  misc_feature 1..20: note = Synthetic
  source 1..20: mol_type = other DNA
ctcaggggcggccctgaggc

SEQ ID NO: 125 - DNA, 20 bp, synthetic construct
  misc_feature 1..20: note = Synthetic
  source 1..20: mol_type = other DNA
ggcccgcttcttggccagct

SEQ ID NO: 126 - DNA, 20 bp, synthetic construct
  misc_feature 1..20: note = Synthetic
  source 1..20: mol_type = other DNA
cgcaccttctccaccaggtc

SEQ ID NO: 127 - DNA, 20 bp, synthetic construct
  misc_feature 1..20: note = Synthetic
  source 1..20: mol_type = other DNA
cgctccttttcttcgcgcgc

SEQ ID NO: 128 - DNA, 20 bp, synthetic construct
  misc_feature 1..20: note = Synthetic
  source 1..20: mol_type = other DNA
cgctctttctcgcgttcccg

SEQ ID NO: 129 - DNA, 20 bp, synthetic construct
  misc_feature 1..20: note = Synthetic
  source 1..20: mol_type = other DNA
tcgcgctctttctcgcgttc

SEQ ID NO: 130 - DNA, 20 bp, synthetic construct
  misc_feature 1..20: note = Synthetic
  source 1..20: mol_type = other DNA
cgctcgcgctctttctcgcg

SEQ ID NO: 131 - DNA, 20 bp, synthetic construct
  misc_feature 1..20: note = Synthetic
  source 1..20: mol_type = other DNA
tcgcgctcgcgctctttctc

SEQ ID NO: 132 - DNA, 20 bp, synthetic construct
  misc_feature 1..20: note = Synthetic
  source 1..20: mol_type = other DNA
ttctcgcgctcgcgctcttt

SEQ ID NO: 133 - DNA, 20 bp, synthetic construct
  misc_feature 1..20: note = Synthetic
  source 1..20: mol_type = other DNA
tccttctcgcgctcgcgctc

SEQ ID NO: 134 - DNA, 20 bp, synthetic construct
  misc_feature 1..20: note = Synthetic
  source 1..20: mol_type = other DNA
cgctccttctcgcgctcgcg

SEQ ID NO: 135 - DNA, 20 bp, synthetic construct
  misc_feature 1..20: note = Synthetic
  source 1..20: mol_type = other DNA
tcgcgctccttctcgcgctc

SEQ ID NO: 136 - DNA, 20 bp, synthetic construct
  misc_feature 1..20: note = Synthetic
  source 1..20: mol_type = other DNA
agctcgcgctccttctcgcg

SEQ ID NO: 137 - DNA, 20 bp, synthetic construct
  misc_feature 1..20: note = Synthetic
  source 1..20: mol_type = other DNA
gagccaacttcacgctgcgt

SEQ ID NO: 138 - DNA, 20 bp, synthetic construct
  misc_feature 1..20: note = Synthetic
  source 1..20: mol_type = other DNA
tgggcccagagatgggcatt

SEQ ID NO: 139 - DNA, 20 bp, synthetic construct
  misc_feature 1..20: note = Synthetic
  source 1..20: mol_type = other DNA
caccgcactgcccggttcaa

SEQ ID NO: 140 - DNA, 20 bp, synthetic construct
  misc_feature 1..20: note = Synthetic
  source 1..20: mol_type = other DNA
agccaccgcactgcccggtt

SEQ ID NO: 141 - DNA, 20 bp, synthetic construct
  misc_feature 1..20: note = Synthetic
  source 1..20: mol_type = other DNA
tgtagccaccgcactgcccg

SEQ ID NO: 142 - DNA, 20 bp, synthetic construct
  misc_feature 1..20: note = Synthetic
  source 1..20: mol_type = other DNA
cactgtagccaccgcactgc

SEQ ID NO: 143 - DNA, 20 bp, synthetic construct
  misc_feature 1..20: note = Synthetic
  source 1..20: mol_type = other DNA
gggcactgtagccaccgcac

SEQ ID NO: 144 - DNA, 20 bp, synthetic construct
  misc_feature 1..20: note = Synthetic
  source 1..20: mol_type = other DNA
gtaggggggcactgtagcca

SEQ ID NO: 145 - DNA, 20 bp, synthetic construct
  misc_feature 1..20: note = Synthetic
  source 1..20: mol_type = other DNA
ggtccactgcccccaggggc

SEQ ID NO: 146 - DNA, 20 bp, synthetic construct
  misc_feature 1..20: note = Synthetic
  source 1..20: mol_type = other DNA
ggggttccagctcactaggc

SEQ ID NO: 147 - DNA, 20 bp, synthetic construct
  misc_feature 1..20: note = Synthetic
  source 1..20: mol_type = other DNA
aaagggtgcaggccaggtgg

SEQ ID NO: 148 - DNA, 20 bp, synthetic construct
  misc_feature 1..20: note = Synthetic
  source 1..20: mol_type = other DNA
gcctctcagctgccagccgc

SEQ ID NO: 149 - DNA, 20 bp, synthetic construct
  misc_feature 1..20: note = Synthetic
  source 1..20: mol_type = other DNA
ccagggccgccaccctttct

SEQ ID NO: 150 - DNA, 20 bp, synthetic construct
  misc_feature 1..20: note = Synthetic
  source 1..20: mol_type = other DNA
taaggggcagcaaagagctg

SEQ ID NO: 151 - DNA, 20 bp, synthetic construct
  misc_feature 1..20: note = Synthetic
  source 1..20: mol_type = other DNA
tgagctgctgacatcggggc

SEQ ID NO: 152 - DNA, 20 bp, synthetic construct
  misc_feature 1..20: note = Synthetic
  source 1..20: mol_type = other DNA
agctcagctgactgtgcgtg

SEQ ID NO: 153 - DNA, 20 bp, synthetic construct
  misc_feature 1..20: note = Synthetic
  source 1..20: mol_type = other DNA
gttccagcgccaagcgctgc

SEQ ID NO: 154 - DNA, 20 bp, synthetic construct
  misc_feature 1..20: note = Synthetic
  source 1..20: mol_type = other DNA
ctcctgggcaggcagcggca

SEQ ID NO: 155 - DNA, 20 bp, synthetic construct
  misc_feature 1..20: note = Synthetic
  source 1..20: mol_type = other DNA
caggccaagggcacggtggg

SEQ ID NO: 156 - DNA, 20 bp, synthetic construct
  misc_feature 1..20: note = Synthetic
  source 1..20: mol_type = other DNA
tggccttctgtctgtccctc

SEQ ID NO: 157 - DNA, 20 bp, synthetic construct
  misc_feature 1..20: note = Synthetic
  source 1..20: mol_type = other DNA
ctgcacaccacatcgggcct

SEQ ID NO: 158 - DNA, 20 bp, synthetic construct
  misc_feature 1..20: note = Synthetic
  source 1..20: mol_type = other DNA
gtccccatcctcgccacctc

SEQ ID NO: 159 - DNA, 20 bp, synthetic construct
  misc_feature 1..20: note = Synthetic
  source 1..20: mol_type = other DNA
aatcacaccaacggggcagg

SEQ ID NO: 160 - DNA, 20 bp, synthetic construct
  misc_feature 1..20: note = Synthetic
  source 1..20: mol_type = other DNA
tggcacacgatgctacgcaa

SEQ ID NO: 161 - DNA, 20 bp, synthetic construct
  misc_feature 1..20: note = Synthetic
  source 1..20: mol_type = other DNA
gggtggcacacgatgctacg

SEQ ID NO: 162 - DNA, 20 bp, synthetic construct
  misc_feature 1..20: note = Synthetic
  source 1..20: mol_type = other DNA
caggggtggcacacgatgct

SEQ ID NO: 163 - DNA, 20 bp, synthetic construct
  misc_feature 1..20: note = Synthetic
  source 1..20: mol_type = other DNA
gggcaggggtggcacacgat

SEQ ID NO: 164 - DNA, 20 bp, synthetic construct
  misc_feature 1..20: note = Synthetic
  source 1..20: mol_type = other DNA
ctactcgggccttcttggct

SEQ ID NO: 165 - DNA, 20 bp, synthetic construct
  misc_feature 1..20: note = Synthetic
  source 1..20: mol_type = other DNA
cctctactcgggccttcttg

SEQ ID NO: 166 - DNA, 20 bp, synthetic construct
  misc_feature 1..20: note = Synthetic
  source 1..20: mol_type = other DNA
cttcctctactcgggccttc

SEQ ID NO: 167 - DNA, 20 bp, synthetic construct
  misc_feature 1..20: note = Synthetic
  source 1..20: mol_type = other DNA
ggctaagaggcggccatagg

SEQ ID NO: 168 - DNA, 20 bp, synthetic construct
  misc_feature 1..20: note = Synthetic
  source 1..20: mol_type = other DNA
gttggctaagaggcggccat

SEQ ID NO: 169 - DNA, 20 bp, synthetic construct
  misc_feature 1..20: note = Synthetic
  source 1..20: mol_type = other DNA
tgttggctaagaggcggcca

SEQ ID NO: 170 - DNA, 20 bp, synthetic construct
  misc_feature 1..20: note = Synthetic
  source 1..20: mol_type = other DNA
gctgttggctaagaggcggc

SEQ ID NO: 171 - DNA, 20 bp, synthetic construct
  misc_feature 1..20: note = Synthetic
  source 1..20: mol_type = other DNA
tgctgttggctaagaggcgg

SEQ ID NO: 172 - DNA, 20 bp, synthetic construct
  misc_feature 1..20: note = Synthetic
  source 1..20: mol_type = other DNA
attgctgttggctaagaggc

SEQ ID NO: 173 - DNA, 20 bp, synthetic construct
  misc_feature 1..20: note = Synthetic
  source 1..20: mol_type = other DNA
cattgctgttggctaagagg

SEQ ID NO: 174 - DNA, 20 bp, synthetic construct
  misc_feature 1..20: note = Synthetic
  source 1..20: mol_type = other DNA
ggcattgctgttggctaaga

SEQ ID NO: 175 - DNA, 20 bp, synthetic construct
  misc_feature 1..20: note = Synthetic
  source 1..20: mol_type = other DNA
gggcattgctgttggctaag

SEQ ID NO: 176 - DNA, 20 bp, synthetic construct
  misc_feature 1..20: note = Synthetic
  source 1..20: mol_type = other DNA
atgggcattgctgttggcta

SEQ ID NO: 177 - DNA, 20 bp, synthetic construct
  misc_feature 1..20: note = Synthetic
  source 1..20: mol_type = other DNA
gatgggcattgctgttggct

SEQ ID NO: 178 - DNA, 20 bp, synthetic construct
  misc_feature 1..20: note = Synthetic
  source 1..20: mol_type = other DNA
tggatgggcattgctgttgg

SEQ ID NO: 179 - DNA, 20 bp, synthetic construct
  misc_feature 1..20: note = Synthetic
  source 1..20: mol_type = other DNA
ctggatgggcattgctgttg

SEQ ID NO: 180 - DNA, 20 bp, synthetic construct
  misc_feature 1..20: note = Synthetic
  source 1..20: mol_type = other DNA
gcctggatgggcattgctgt

SEQ ID NO: 181 - DNA, 20 bp, synthetic construct
  misc_feature 1..20: note = Synthetic
  source 1..20: mol_type = other DNA
ggcctggatgggcattgctg

SEQ ID NO: 182 - DNA, 20 bp, synthetic construct
  misc_feature 1..20: note = Synthetic
  source 1..20: mol_type = other DNA
ggggcctggatgggcattgc

SEQ ID NO: 183 - DNA, 20 bp, synthetic construct
  misc_feature 1..20: note = Synthetic
  source 1..20: mol_type = other DNA
aggtgggggcaggtgtgctg

SEQ ID NO: 184 - DNA, 20 bp, synthetic construct
  misc_feature 1..20: note = Synthetic
  source 1..20: mol_type = other DNA
gaggtgggggcaggtgtgct

SEQ ID NO: 185 - DNA, 20 bp, synthetic construct
  misc_feature 1..20: note = Synthetic
  source 1..20: mol_type = other DNA
cgaggaagccacggtggcaa

SEQ ID NO: 186 - DNA, 20 bp, synthetic construct
  misc_feature 1..20: note = Synthetic
  source 1..20: mol_type = other DNA
ccttctccaccaggtcggcc

SEQ ID NO: 187 - DNA, 20 bp, synthetic construct
  misc_feature 1..20: note = Synthetic
  source 1..20: mol_type = other DNA
gcgccgcaccttctccacca

SEQ ID NO: 188 - DNA, 20 bp, synthetic construct
  misc_feature 1..20: note = Synthetic
  source 1..20: mol_type = other DNA
ctctttctcgcgttcccgct

SEQ ID NO: 189 - DNA, 20 bp, synthetic construct
  misc_feature 1..20: note = Synthetic
  source 1..20: mol_type = other DNA
gctctttctcgcgttcccgc

SEQ ID NO: 190 - DNA, 20 bp, synthetic construct
  misc_feature 1..20: note = Synthetic
  source 1..20: mol_type = other DNA
gcgctctttctcgcgttccc

SEQ ID NO: 191 - DNA, 20 bp, synthetic construct
  misc_feature 1..20: note = Synthetic
  source 1..20: mol_type = other DNA
cgcgctctttctcgcgttcc

SEQ ID NO: 192 - DNA, 20 bp, synthetic construct
  misc_feature 1..20: note = Synthetic
  source 1..20: mol_type = other DNA
ctcgcgctctttctcgcgtt

SEQ ID NO: 193 - DNA, 20 bp, synthetic construct
  misc_feature 1..20: note = Synthetic
  source 1..20: mol_type = other DNA
gctcgcgctctttctcgcgt

SEQ ID NO: 194 - DNA, 20 bp, synthetic construct
  misc_feature 1..20: note = Synthetic
  source 1..20: mol_type = other DNA
gcgctcgcgctctttctcgc

SEQ ID NO: 195 - DNA, 20 bp, synthetic construct
  misc_feature 1..20: note = Synthetic
  source 1..20: mol_type = other DNA
cgcgctcgcgctctttctcg

SEQ ID NO: 196 - DNA, 20 bp, synthetic construct
  misc_feature 1..20: note = Synthetic
  source 1..20: mol_type = other DNA
ctcgcgctcgcgctctttct

SEQ ID NO: 197 - DNA, 20 bp, synthetic construct
  misc_feature 1..20: note = Synthetic
  source 1..20: mol_type = other DNA
tctcgcgctcgcgctctttc

SEQ ID NO: 198 - DNA, 20 bp, synthetic construct
  misc_feature 1..20: note = Synthetic
  source 1..20: mol_type = other DNA
cttctcgcgctcgcgctctt

SEQ ID NO: 199 - DNA, 20 bp, synthetic construct
  misc_feature 1..20: note = Synthetic
  source 1..20: mol_type = other DNA
ccttctcgcgctcgcgctct

SEQ ID NO: 200 - DNA, 20 bp, synthetic construct
  misc_feature 1..20: note = Synthetic
  source 1..20: mol_type = other DNA
ctccttctcgcgctcgcgct

SEQ ID NO: 201 - DNA, 20 bp, synthetic construct
  misc_feature 1..20: note = Synthetic
  source 1..20: mol_type = other DNA
gctccttctcgcgctcgcgc

SEQ ID NO: 202 - DNA, 20 bp, synthetic construct
  misc_feature 1..20: note = Synthetic
  source 1..20: mol_type = other DNA
gcgctccttctcgcgctcgc

SEQ ID NO: 203 - DNA, 20 bp, synthetic construct
  misc_feature 1..20: note = Synthetic
  source 1..20: mol_type = other DNA
cgcgctccttctcgcgctcg

SEQ ID NO: 204 - DNA, 20 bp, synthetic construct
  misc_feature 1..20: note = Synthetic
  source 1..20: mol_type = other DNA
gcactgtagccaccgcactg

SEQ ID NO: 205 - DNA, 20 bp, synthetic construct
  misc_feature 1..20: note = Synthetic
  source 1..20: mol_type = other DNA
ggcactgtagccaccgcact

SEQ ID NO: 206 - DNA, 20 bp, synthetic construct
  misc_feature 1..20: note = Synthetic
  source 1..20: mol_type = other DNA
ggggcactgtagccaccgca

SEQ ID NO: 207 - DNA, 20 bp, synthetic construct
  misc_feature 1..20: note = Synthetic
  source 1..20: mol_type = other DNA
gggggcactgtagccaccgc

SEQ ID NO: 208 - DNA, 20 bp, synthetic construct
  misc_feature 1..20: note = Synthetic
  source 1..20: mol_type = other DNA
ggggggcactgtagccaccg

SEQ ID NO: 209 - DNA, 20 bp, synthetic construct
  misc_feature 1..20: note = Synthetic
  source 1..20: mol_type = other DNA
ccgggtccactgcccccagg

SEQ ID NO: 210 - DNA, 20 bp, synthetic construct
  misc_feature 1..20: note = Synthetic
  source 1..20: mol_type = other DNA
tgctgcctctcagctgccag

SEQ ID NO: 211 - DNA, 20 bp, synthetic construct
  misc_feature 1..20: note = Synthetic
  source 1..20: mol_type = other DNA
tgcgtgctgcctctcagctg

SEQ ID NO: 212 - DNA, 20 bp, synthetic construct
  misc_feature 1..20: note = Synthetic
  source 1..20: mol_type = other DNA
ctgctgacatcggggcagaa

SEQ ID NO: 213 - DNA, 20 bp, synthetic construct
  misc_feature 1..20: note = Synthetic
  source 1..20: mol_type = other DNA
ctgatgagctgctgacatcg

SEQ ID NO: 214 - DNA, 20 bp, synthetic construct
  misc_feature 1..20: note = Synthetic
  source 1..20: mol_type = other DNA
cagcgccaagcgctgcagct

SEQ ID NO: 215 - DNA, 20 bp, synthetic construct
  misc_feature 1..20: note = Synthetic
  source 1..20: mol_type = other DNA
tgctgttccagcgccaagcg

SEQ ID NO: 216 - DNA, 20 bp, synthetic construct
  misc_feature 1..20: note = Synthetic
  source 1..20: mol_type = other DNA
gggcaggcagcggcacactg

SEQ ID NO: 217 - DNA, 20 bp, synthetic construct
  misc_feature 1..20: note = Synthetic
  source 1..20: mol_type = other DNA
acaccacatcgggccttggc

SEQ ID NO: 218 - DNA, 20 bp, synthetic construct
  misc_feature 1..20: note = Synthetic
  source 1..20: mol_type = other DNA
acctctgcacaccacatcgg

SEQ ID NO: 219 - DNA, 20 bp, synthetic construct
  misc_feature 1..20: note = Synthetic
  source 1..20: mol_type = other DNA
gcaggggtggcacacgatgc

SEQ ID NO: 220 - DNA, 20 bp, synthetic construct
  misc_feature 1..20: note = Synthetic
  source 1..20: mol_type = other DNA
ggcaggggtggcacacgatg

SEQ ID NO: 221 - DNA, 20 bp, synthetic construct
  misc_feature 1..20: note = Synthetic
  source 1..20: mol_type = other DNA
ggggcaggggtggcacacga

SEQ ID NO: 222 - DNA, 20 bp, synthetic construct
  misc_feature 1..20: note = Synthetic
  source 1..20: mol_type = other DNA
gggcgcgcacacagggatcg

SEQ ID NO: 223 - DNA, 20 bp, synthetic construct
  misc_feature 1..20: note = Synthetic
  source 1..20: mol_type = other DNA
actcgggccttcttggctgt

SEQ ID NO: 224 - DNA, 20 bp, synthetic construct
  misc_feature 1..20: note = Synthetic
  source 1..20: mol_type = other DNA
tactcgggccttcttggctg

SEQ ID NO: 225 - DNA, 20 bp, synthetic construct
  misc_feature 1..20: note = Synthetic
  source 1..20: mol_type = other DNA
tctactcgggccttcttggc

SEQ ID NO: 226 - DNA, 20 bp, synthetic construct
  misc_feature 1..20: note = Synthetic
  source 1..20: mol_type = other DNA
ctctactcgggccttcttgg

SEQ ID NO: 227 - DNA, 20 bp, synthetic construct
  misc_feature 1..20: note = Synthetic
  source 1..20: mol_type = other DNA
tcctctactcgggccttctt

SEQ ID NO: 228 - DNA, 20 bp, synthetic construct
  misc_feature 1..20: note = Synthetic
  source 1..20: mol_type = other DNA
ttcctctactcgggccttct

SEQ ID NO: 229 - DNA, 20 bp, synthetic construct
  misc_feature 1..20: note = Synthetic
  source 1..20: mol_type = other DNA
gcttcctctactcgggcctt

SEQ ID NO: 230 - DNA, 20 bp, synthetic construct
  misc_feature 1..20: note = Synthetic
  source 1..20: mol_type = other DNA
ggcttcctctactcgggcct

SEQ ID NO: 231 - DNA, 20 bp, synthetic construct
  misc_feature 1..20: note = Synthetic
  source 1..20: mol_type = other DNA
ctaagaggcggccataggga

SEQ ID NO: 232 - DNA, 20 bp, synthetic construct
  misc_feature 1..20: note = Synthetic
  source 1..20: mol_type = other DNA
gctaagaggcggccataggg

SEQ ID NO: 233 - DNA, 20 bp, synthetic construct
  misc_feature 1..20: note = Synthetic
  source 1..20: mol_type = other DNA
tggctaagaggcggccatag

SEQ ID NO: 234 - DNA, 20 bp, synthetic construct
  misc_feature 1..20: note = Synthetic
  source 1..20: mol_type = other DNA
ttggctaagaggcggccata

SEQ ID NO: 235 - DNA, 20 bp, synthetic construct
  misc_feature 1..20: note = Synthetic
  source 1..20: mol_type = other DNA
gtgaggtgggggcaggtgtg

SEQ ID NO: 236 - DNA, 20 bp, synthetic construct
  misc_feature 1..20: note = Synthetic
  source 1..20: mol_type = other DNA
tgtgaggtgggggcaggtgt

SEQ ID NO: 237 - DNA, 20 bp, synthetic construct
  misc_feature 1..20: note = Synthetic
  source 1..20: mol_type = other DNA
gctgtgaggtgggggcaggt

SEQ ID NO: 238 - DNA, 20 bp, synthetic construct
  misc_feature 1..20: note = Synthetic
  source 1..20: mol_type = other DNA
ggctgtgaggtgggggcagg

SEQ ID NO: 239 - DNA, 20 bp, synthetic construct
  misc_feature 1..20: note = Synthetic
  source 1..20: mol_type = other DNA
ctggctgtgaggtgggggca

SEQ ID NO: 240 - DNA, 20 bp, synthetic construct
  misc_feature 1..20: note = Synthetic
  source 1..20: mol_type = other DNA
gaggaagccacggtggcaat

SEQ ID NO: 241 - DNA, 20 bp, synthetic construct
  misc_feature 1..20: note = Synthetic
  source 1..20: mol_type = other DNA
cggcccgcttcttggccagc

SEQ ID NO: 242 - DNA, 20 bp, synthetic construct
  misc_feature 1..20: note = Synthetic
  source 1..20: mol_type = other DNA
tcggcccgcttcttggccag

SEQ ID NO: 243 - DNA, 20 bp, synthetic construct
  misc_feature 1..20: note = Synthetic
  source 1..20: mol_type = other DNA
gtcggcccgcttcttggcca

SEQ ID NO: 244 - DNA, 20 bp, synthetic construct
  misc_feature 1..20: note = Synthetic
  source 1..20: mol_type = other DNA
ggtcggcccgcttcttggcc

SEQ ID NO: 245 - DNA, 20 bp, synthetic construct
  misc_feature 1..20: note = Synthetic
  source 1..20: mol_type = other DNA
aggtcggcccgcttcttggc

SEQ ID NO: 246 - DNA, 20 bp, synthetic construct
  misc_feature 1..20: note = Synthetic
  source 1..20: mol_type = other DNA
caggtcggcccgcttcttgg

SEQ ID NO: 247 - DNA, 20 bp, synthetic construct
  misc_feature 1..20: note = Synthetic
  source 1..20: mol_type = other DNA
ccaggtcggcccgcttcttg

SEQ ID NO: 248 - DNA, 20 bp, synthetic construct
  misc_feature 1..20: note = Synthetic
  source 1..20: mol_type = other DNA
accaggtcggcccgcttctt

SEQ ID NO: 249 - DNA, 20 bp, synthetic construct
  misc_feature 1..20: note = Synthetic
  source 1..20: mol_type = other DNA
caccaggtcggcccgcttct

SEQ ID NO: 250 - DNA, 20 bp, synthetic construct
  misc_feature 1..20: note = Synthetic
  source 1..20: mol_type = other DNA
ccaccaggtcggcccgcttc

SEQ ID NO: 251 - DNA, 20 bp, synthetic construct
  misc_feature 1..20: note = Synthetic
  source 1..20: mol_type = other DNA
tccaccaggtcggcccgctt

SEQ ID NO: 252 - DNA, 20 bp, synthetic construct
  misc_feature 1..20: note = Synthetic
  source 1..20: mol_type = other DNA
ctccaccaggtcggcccgct

SEQ ID NO: 253 - DNA, 20 bp, synthetic construct
  misc_feature 1..20: note = Synthetic
  source 1..20: mol_type = other DNA
tctccaccaggtcggcccgc

SEQ ID NO: 254 - DNA, 20 bp, synthetic construct
  misc_feature 1..20: note = Synthetic
  source 1..20: mol_type = other DNA
ttctccaccaggtcggcccg

SEQ ID NO: 255 - DNA, 20 bp, synthetic construct
  misc_feature 1..20: note = Synthetic
  source 1..20: mol_type = other DNA
cttctccaccaggtcggccc

SEQ ID NO: 256 - DNA, 20 bp, synthetic construct
  misc_feature 1..20: note = Synthetic
  source 1..20: mol_type = other DNA
accttctccaccaggtcggc

SEQ ID NO: 257 - DNA, 20 bp, synthetic construct
  misc_feature 1..20: note = Synthetic
  source 1..20: mol_type = other DNA
caccttctccaccaggtcgg

SEQ ID NO: 258 - DNA, 20 bp, synthetic construct
  misc_feature 1..20: note = Synthetic
  source 1..20: mol_type = other DNA
gcaccttctccaccaggtcg

SEQ ID NO: 259 - DNA, 20 bp, synthetic construct
  misc_feature 1..20: note = Synthetic
  source 1..20: mol_type = other DNA
ccgcaccttctccaccaggt

SEQ ID NO: 260 - DNA, 20 bp, synthetic construct
  misc_feature 1..20: note = Synthetic
  source 1..20: mol_type = other DNA
gccgcaccttctccaccagg

SEQ ID NO: 261 - DNA, 20 bp, synthetic construct
  misc_feature 1..20: note = Synthetic
  source 1..20: mol_type = other DNA
cgccgcaccttctccaccag

SEQ ID NO: 262 - DNA, 20 bp, synthetic construct
  misc_feature 1..20: note = Synthetic
  source 1..20: mol_type = other DNA
cgcgccgcaccttctccacc

SEQ ID NO: 263 - DNA, 20 bp, synthetic construct
  misc_feature 1..20: note = Synthetic
  source 1..20: mol_type = other DNA
tcgcgccgcaccttctccac

SEQ ID NO: 264 - DNA, 20 bp, synthetic construct
  misc_feature 1..20: note = Synthetic
  source 1..20: mol_type = other DNA
ctcgcgccgcaccttctcca

SEQ ID NO: 265 - DNA, 20 bp, synthetic construct
  misc_feature 1..20: note = Synthetic
  source 1..20: mol_type = other DNA
cctcgcgccgcaccttctcc

SEQ ID NO: 266 - DNA, 20 bp, synthetic construct
  misc_feature 1..20: note = Synthetic
  source 1..20: mol_type = other DNA
gcctcgcgccgcaccttctc

SEQ ID NO: 267 - DNA, 20 bp, synthetic construct
  misc_feature 1..20: note = Synthetic
  source 1..20: mol_type = other DNA
ggcctcgcgccgcaccttct

SEQ ID NO: 268 - DNA, 20 bp, synthetic construct
  misc_feature 1..20: note = Synthetic
  source 1..20: mol_type = other DNA
cggcctcgcgccgcaccttc

SEQ ID NO: 269 - DNA, 20 bp, synthetic construct
  misc_feature 1..20: note = Synthetic
  source 1..20: mol_type = other DNA
tcggcctcgcgccgcacctt

SEQ ID NO: 270 - DNA, 20 bp, synthetic construct
  misc_feature 1..20: note = Synthetic
  source 1..20: mol_type = other DNA
ctcggcctcgcgccgcacct

SEQ ID NO: 271 - DNA, 20 bp, synthetic construct
  misc_feature 1..20: note = Synthetic
  source 1..20: mol_type = other DNA
gctcggcctcgcgccgcacc

SEQ ID NO: 272 - DNA, 20 bp, synthetic construct
  misc_feature 1..20: note = Synthetic
  source 1..20: mol_type = other DNA
tgctcggcctcgcgccgcac

SEQ ID NO: 273 - DNA, 20 bp, synthetic construct
  misc_feature 1..20: note = Synthetic
  source 1..20: mol_type = other DNA
gcgctccttttcttcgcgcg

SEQ ID NO: 274 - DNA, 20 bp, synthetic construct
  misc_feature 1..20: note = Synthetic
  source 1..20: mol_type = other DNA
cgcgctccttttcttcgcgc

SEQ ID NO: 275 - DNA, 20 bp, synthetic construct
  misc_feature 1..20: note = Synthetic
  source 1..20: mol_type = other DNA
tcgcgctccttttcttcgcg

SEQ ID NO: 276 - DNA, 20 bp, synthetic construct
  misc_feature 1..20: note = Synthetic
  source 1..20: mol_type = other DNA
ctcgcgctccttttcttcgc

SEQ ID NO: 277 - DNA, 20 bp, synthetic construct
  misc_feature 1..20: note = Synthetic
  source 1..20: mol_type = other DNA
gctcgcgctccttttcttcg

SEQ ID NO: 278 - DNA, 20 bp, synthetic construct
  misc_feature 1..20: note = Synthetic
  source 1..20: mol_type = other DNA
cgctcgcgctccttttcttc

SEQ ID NO: 279 - DNA, 20 bp, synthetic construct
  misc_feature 1..20: note = Synthetic
  source 1..20: mol_type = other DNA
gcgctcgcgctccttttctt

SEQ ID NO: 280 - DNA, 20 bp, synthetic construct
  misc_feature 1..20: note = Synthetic
  source 1..20: mol_type = other DNA
cgcgctcgcgctccttttct

SEQ ID NO: 281 - DNA, 20 bp, synthetic construct
  misc_feature 1..20: note = Synthetic
  source 1..20: mol_type = other DNA
tcgcgctcgcgctccttttc

SEQ ID NO: 282 - DNA, 20 bp, synthetic construct
  misc_feature 1..20: note = Synthetic
  source 1..20: mol_type = other DNA
ctcgcgctcgcgctcctttt

SEQ ID NO: 283 - DNA, 20 bp, synthetic construct
  misc_feature 1..20: note = Synthetic
  source 1..20: mol_type = other DNA
gctcgcgctcgcgctccttt

SEQ ID NO: 284 - DNA, 20 bp, synthetic construct
  misc_feature 1..20: note = Synthetic
  source 1..20: mol_type = other DNA
cgctcgcgctcgcgctcctt

SEQ ID NO: 285 - DNA, 20 bp, synthetic construct
  misc_feature 1..20: note = Synthetic
  source 1..20: mol_type = other DNA
ccgctcgcgctcgcgctcct

SEQ ID NO: 286 - DNA, 20 bp, synthetic construct
  misc_feature 1..20: note = Synthetic
  source 1..20: mol_type = other DNA
cccgctcgcgctcgcgctcc

SEQ ID NO: 287 - DNA, 20 bp, synthetic construct
  misc_feature 1..20: note = Synthetic
  source 1..20: mol_type = other DNA
tcccgctcgcgctcgcgctc

SEQ ID NO: 288 - DNA, 20 bp, synthetic construct
  misc_feature 1..20: note = Synthetic
  source 1..20: mol_type = other DNA
ttcccgctcgcgctcgcgct

SEQ ID NO: 289 - DNA, 20 bp, synthetic construct
  misc_feature 1..20: note = Synthetic
  source 1..20: mol_type = other DNA
gttcccgctcgcgctcgcgc

SEQ ID NO: 290 - DNA, 20 bp, synthetic construct
  misc_feature 1..20: note = Synthetic
  source 1..20: mol_type = other DNA
cgttcccgctcgcgctcgcg

SEQ ID NO: 291 - DNA, 20 bp, synthetic construct
  misc_feature 1..20: note = Synthetic
  source 1..20: mol_type = other DNA
gcgttcccgctcgcgctcgc

SEQ ID NO: 292 - DNA, 20 bp, synthetic construct
  misc_feature 1..20: note = Synthetic
  source 1..20: mol_type = other DNA
cgcgttcccgctcgcgctcg

SEQ ID NO: 293 - DNA, 20 bp, synthetic construct
  misc_feature 1..20: note = Synthetic
  source 1..20: mol_type = other DNA
tcgcgttcccgctcgcgctc

SEQ ID NO: 294 - DNA, 20 bp, synthetic construct
  misc_feature 1..20: note = Synthetic
  source 1..20: mol_type = other DNA
ctcgcgttcccgctcgcgct

SEQ ID NO: 295 - DNA, 20 bp, synthetic construct
  misc_feature 1..20: note = Synthetic
  source 1..20: mol_type = other DNA
tctcgcgttcccgctcgcgc

SEQ ID NO: 296 - DNA, 20 bp, synthetic construct
  misc_feature 1..20: note = Synthetic
  source 1..20: mol_type = other DNA
ttctcgcgttcccgctcgcg

SEQ ID NO: 297 - DNA, 20 bp, synthetic construct
  misc_feature 1..20: note = Synthetic
  source 1..20: mol_type = other DNA
tttctcgcgttcccgctcgc

SEQ ID NO: 298 - DNA, 20 bp, synthetic construct
  misc_feature 1..20: note = Synthetic
  source 1..20: mol_type = other DNA
ctttctcgcgttcccgctcg

SEQ ID NO: 299 - DNA, 20 bp, synthetic construct
  misc_feature 1..20: note = Synthetic
  source 1..20: mol_type = other DNA
ctcgcgctccttctcgcgct

SEQ ID NO: 300 - DNA, 20 bp, synthetic construct
  misc_feature 1..20: note = Synthetic
  source 1..20: mol_type = other DNA
gctcgcgctccttctcgcgc

SEQ ID NO: 301 - DNA, 20 bp, synthetic construct
  misc_feature 1..20: note = Synthetic
  source 1..20: mol_type = other DNA
aagctcgcgctccttctcgc

SEQ ID NO: 302 - DNA, 20 bp, synthetic construct
  misc_feature 1..20: note = Synthetic
  source 1..20: mol_type = other DNA
caagctcgcgctccttctcg

SEQ ID NO: 303 - DNA, 20 bp, synthetic construct
  misc_feature 1..20: note = Synthetic
  source 1..20: mol_type = other DNA
ttcaagctcgcgctccttct

SEQ ID NO: 304 - DNA, 20 bp, synthetic construct
  misc_feature 1..20: note = Synthetic
  source 1..20: mol_type = other DNA
gttcaagctcgcgctccttc

SEQ ID NO: 305 - DNA, 20 bp, synthetic construct
  misc_feature 1..20: note = Synthetic
  source 1..20: mol_type = other DNA
cgttcaagctcgcgctcctt

SEQ ID NO: 306 - DNA, 20 bp, synthetic construct
  misc_feature 1..20: note = Synthetic
  source 1..20: mol_type = other DNA
gcgttcaagctcgcgctcct

SEQ ID NO: 307 - DNA, 20 bp, synthetic construct
  misc_feature 1..20: note = Synthetic
  source 1..20: mol_type = other DNA
ccgcactgcccggttcaaat

SEQ ID NO: 308 - DNA, 20 bp, synthetic construct
  misc_feature 1..20: note = Synthetic
  source 1..20: mol_type = other DNA
accgcactgcccggttcaaa

SEQ ID NO: 309 - DNA, 20 bp, synthetic construct
  misc_feature 1..20: note = Synthetic
  source 1..20: mol_type = other DNA
ccaccgcactgcccggttca

SEQ ID NO: 310 - DNA, 20 bp, synthetic construct
  misc_feature 1..20: note = Synthetic
  source 1..20: mol_type = other DNA
gccaccgcactgcccggttc

SEQ ID NO: 311 - DNA, 20 bp, synthetic construct
  misc_feature 1..20: note = Synthetic
  source 1..20: mol_type = other DNA
tagccaccgcactgcccggt

SEQ ID NO: 312 - DNA, 20 bp, synthetic construct
  misc_feature 1..20: note = Synthetic
  source 1..20: mol_type = other DNA
gtagccaccgcactgcccgg

SEQ ID NO: 313 - DNA, 20 bp, synthetic construct
  misc_feature 1..20: note = Synthetic
  source 1..20: mol_type = other DNA
ctgtagccaccgcactgccc

SEQ ID NO: 314 - DNA, 20 bp, synthetic construct
  misc_feature 1..20: note = Synthetic
  source 1..20: mol_type = other DNA
actgtagccaccgcactgcc

SEQ ID NO: 315 - DNA, 20 bp, synthetic construct
  misc_feature 1..20: note = Synthetic
  source 1..20: mol_type = other DNA
cccgggtccactgcccccag

SEQ ID NO: 316 - DNA, 20 bp, synthetic construct
  misc_feature 1..20: note = Synthetic
  source 1..20: mol_type = other DNA
gtgctgcctctcagctgcca

SEQ ID NO: 317 - DNA, 20 bp, synthetic construct
  misc_feature 1..20: note = Synthetic
  source 1..20: mol_type = other DNA
cgtgctgcctctcagctgcc

SEQ ID NO: 318 - DNA, 20 bp, synthetic construct
  misc_feature 1..20: note = Synthetic
  source 1..20: mol_type = other DNA
gcgtgctgcctctcagctgc

SEQ ID NO: 319 - DNA, 20 bp, synthetic construct
  misc_feature 1..20: note = Synthetic
  source 1..20: mol_type = other DNA
ctgcgtgctgcctctcagct

SEQ ID NO: 320 - DNA, 20 bp, synthetic construct
  misc_feature 1..20: note = Synthetic
  source 1..20: mol_type = other DNA
tctgcgtgctgcctctcagc

SEQ ID NO: 321 - DNA, 20 bp, synthetic construct
  misc_feature 1..20: note = Synthetic
  source 1..20: mol_type = other DNA
ttctgcgtgctgcctctcag

SEQ ID NO: 322 - DNA, 20 bp, synthetic construct
  misc_feature 1..20: note = Synthetic
  source 1..20: mol_type = other DNA
tttctgcgtgctgcctctca

SEQ ID NO: 323 - DNA, 20 bp, synthetic construct
  misc_feature 1..20: note = Synthetic
  source 1..20: mol_type = other DNA
ctttctgcgtgctgcctctc

SEQ ID NO: 324 - DNA, 20 bp, synthetic construct
  misc_feature 1..20: note = Synthetic
  source 1..20: mol_type = other DNA
cctttctgcgtgctgcctct

SEQ ID NO: 325 - DNA, 20 bp, synthetic construct
  misc_feature 1..20: note = Synthetic
  source 1..20: mol_type = other DNA
ccctttctgcgtgctgcctc

SEQ ID NO: 326 - DNA, 20 bp, synthetic construct
  misc_feature 1..20: note = Synthetic
  source 1..20: mol_type = other DNA
accctttctgcgtgctgcct

SEQ ID NO: 327 - DNA, 20 bp, synthetic construct
  misc_feature 1..20: note = Synthetic
  source 1..20: mol_type = other DNA
caccctttctgcgtgctgcc

SEQ ID NO: 328 - DNA, 20 bp, synthetic construct
  misc_feature 1..20: note = Synthetic
  source 1..20: mol_type = other DNA
ccaccctttctgcgtgctgc

SEQ ID NO: 329 - DNA, 20 bp, synthetic construct
  misc_feature 1..20: note = Synthetic
  source 1..20: mol_type = other DNA
gccaccctttctgcgtgctg

SEQ ID NO: 330 - DNA, 20 bp, synthetic construct
  misc_feature 1..20: note = Synthetic
  source 1..20: mol_type = other DNA
cgccaccctttctgcgtgct

SEQ ID NO: 331 - DNA, 20 bp, synthetic construct
  misc_feature 1..20: note = Synthetic
  source 1..20: mol_type = other DNA
ccgccaccctttctgcgtgc

SEQ ID NO: 332 - DNA, 20 bp, synthetic construct
  misc_feature 1..20: note = Synthetic
  source 1..20: mol_type = other DNA
gccgccaccctttctgcgtg

SEQ ID NO: 333 - DNA, 20 bp, synthetic construct
  misc_feature 1..20: note = Synthetic
  source 1..20: mol_type = other DNA
ggccgccaccctttctgcgt

SEQ ID NO: 334 - DNA, 20 bp, synthetic construct
  misc_feature 1..20: note = Synthetic
  source 1..20: mol_type = other DNA
gggccgccaccctttctgcg

SEQ ID NO: 335 - DNA, 20 bp, synthetic construct
  misc_feature 1..20: note = Synthetic
  source 1..20: mol_type = other DNA
agggccgccaccctttctgc

SEQ ID NO: 336 - DNA, 20 bp, synthetic construct
  misc_feature 1..20: note = Synthetic
  source 1..20: mol_type = other DNA
cagggccgccaccctttctg

SEQ ID NO: 337 - DNA, 20 bp, synthetic construct
  misc_feature 1..20: note = Synthetic
  source 1..20: mol_type = other DNA
ggcagaaagggaggccggca

SEQ ID NO: 338 - DNA, 20 bp, synthetic construct
  misc_feature 1..20: note = Synthetic
  source 1..20: mol_type = other DNA
gggcagaaagggaggccggc

SEQ ID NO: 339 - DNA, 20 bp, synthetic construct
  misc_feature 1..20: note = Synthetic
  source 1..20: mol_type = other DNA
ggggcagaaagggaggccgg

SEQ ID NO: 340 - DNA, 20 bp, synthetic construct
  misc_feature 1..20: note = Synthetic
  source 1..20: mol_type = other DNA
cggggcagaaagggaggccg

SEQ ID NO: 341 - DNA, 20 bp, synthetic construct
  misc_feature 1..20: note = Synthetic
  source 1..20: mol_type = other DNA
tcggggcagaaagggaggcc

SEQ ID NO: 342 - DNA, 20 bp, synthetic construct
  misc_feature 1..20: note = Synthetic
  source 1..20: mol_type = other DNA
atcggggcagaaagggaggc

SEQ ID NO: 343 - DNA, 20 bp, synthetic construct
  misc_feature 1..20: note = Synthetic
  source 1..20: mol_type = other DNA
catcggggcagaaagggagg

SEQ ID NO: 344 - DNA, 20 bp, synthetic construct
  misc_feature 1..20: note = Synthetic
  source 1..20: mol_type = other DNA
acatcggggcagaaagggag

SEQ ID NO: 345 - DNA, 20 bp, synthetic construct
  misc_feature 1..20: note = Synthetic
  source 1..20: mol_type = other DNA
gacatcggggcagaaaggga

SEQ ID NO: 346 - DNA, 20 bp, synthetic construct
  misc_feature 1..20: note = Synthetic
  source 1..20: mol_type = other DNA
tgacatcggggcagaaaggg

SEQ ID NO: 347 - DNA, 20 bp, synthetic construct
  misc_feature 1..20: note = Synthetic
  source 1..20: mol_type = other DNA
ctgacatcggggcagaaagg

SEQ ID NO: 348 - DNA, 20 bp, synthetic construct
  misc_feature 1..20: note = Synthetic
  source 1..20: mol_type = other DNA
gctgacatcggggcagaaag

SEQ ID NO: 349 - DNA, 20 bp, synthetic construct
  misc_feature 1..20: note = Synthetic
  source 1..20: mol_type = other DNA
tgctgacatcggggcagaaa

SEQ ID NO: 350 - DNA, 20 bp, synthetic construct
  misc_feature 1..20: note = Synthetic
  source 1..20: mol_type = other DNA
gctgctgacatcggggcaga

SEQ ID NO: 351 - DNA, 20 bp, synthetic construct
  misc_feature 1..20: note = Synthetic
  source 1..20: mol_type = other DNA
agctgctgacatcggggcag

SEQ ID NO: 352 - DNA, 20 bp, synthetic construct
  misc_feature 1..20: note = Synthetic
  source 1..20: mol_type = other DNA
gagctgctgacatcggggca

SEQ ID NO: 353 - DNA, 20 bp, synthetic construct
  misc_feature 1..20: note = Synthetic
  source 1..20: mol_type = other DNA
atgagctgctgacatcgggg

SEQ ID NO: 354 - DNA, 20 bp, synthetic construct
  misc_feature 1..20: note = Synthetic
  source 1..20: mol_type = other DNA
gatgagctgctgacatcggg

SEQ ID NO: 355 - DNA, 20 bp, synthetic construct
  misc_feature 1..20: note = Synthetic
  source 1..20: mol_type = other DNA
tgatgagctgctgacatcgg

SEQ ID NO: 356 - DNA, 20 bp, synthetic construct
  misc_feature 1..20: note = Synthetic
  source 1..20: mol_type = other DNA
gctgatgagctgctgacatc

SEQ ID NO: 357 - DNA, 20 bp, synthetic construct
  misc_feature 1..20: note = Synthetic
  source 1..20: mol_type = other DNA
agctgatgagctgctgacat

SEQ ID NO: 358 - DNA, 20 bp, synthetic construct
  misc_feature 1..20: note = Synthetic
  source 1..20: mol_type = other DNA
cagctgatgagctgctgaca

SEQ ID NO: 359 - DNA, 20 bp, synthetic construct
  misc_feature 1..20: note = Synthetic
  source 1..20: mol_type = other DNA
gcagctgatgagctgctgac

SEQ ID NO: 360 - DNA, 20 bp, synthetic construct
  misc_feature 1..20: note = Synthetic
  source 1..20: mol_type = other DNA
tgcagctgatgagctgctga

SEQ ID NO: 361 - DNA, 20 bp, synthetic construct
  misc_feature 1..20: note = Synthetic
  source 1..20: mol_type = other DNA
ctgcagctgatgagctgctg

SEQ ID NO: 362 - DNA, 20 bp, synthetic construct
  misc_feature 1..20: note = Synthetic
  source 1..20: mol_type = other DNA
cctgcagctgatgagctgct

SEQ ID NO: 363 - DNA, 20 bp, synthetic construct
  misc_feature 1..20: note = Synthetic
  source 1..20: mol_type = other DNA
gcctgcagctgatgagctgc

SEQ ID NO: 364 - DNA, 20 bp, synthetic construct
  misc_feature 1..20: note = Synthetic
  source 1..20: mol_type = other DNA
ggcctgcagctgatgagctg

SEQ ID NO: 365 - DNA, 20 bp, synthetic construct
  misc_feature 1..20: note = Synthetic
  source 1..20: mol_type = other DNA
tggcctgcagctgatgagct

SEQ ID NO: 366 - DNA, 20 bp, synthetic construct
  misc_feature 1..20: note = Synthetic
  source 1..20: mol_type = other DNA
atggcctgcagctgatgagc

SEQ ID NO: 367 - DNA, 20 bp, synthetic construct
  misc_feature 1..20: note = Synthetic
  source 1..20: mol_type = other DNA
catggcctgcagctgatgag

SEQ ID NO: 368 - DNA, 20 bp, synthetic construct
  misc_feature 1..20: note = Synthetic
  source 1..20: mol_type = other DNA
gcatggcctgcagctgatga

SEQ ID NO: 369 - DNA, 20 bp, synthetic construct
  misc_feature 1..20: note = Synthetic
  source 1..20: mol_type = other DNA
tgcatggcctgcagctgatg

SEQ ID NO: 370 - DNA, 20 bp, synthetic construct
  misc_feature 1..20: note = Synthetic
  source 1..20: mol_type = other DNA
cagctcagctgactgtgcgt

SEQ ID NO: 371 - DNA, 20 bp, synthetic construct
  misc_feature 1..20: note = Synthetic
  source 1..20: mol_type = other DNA
gcagctcagctgactgtgcg

SEQ ID NO: 372 - DNA, 20 bp, synthetic construct
  misc_feature 1..20: note = Synthetic
  source 1..20: mol_type = other DNA
tgcagctcagctgactgtgc

SEQ ID NO: 373 - DNA, 20 bp, synthetic construct
  misc_feature 1..20: note = Synthetic
  source 1..20: mol_type = other DNA
ctgcagctcagctgactgtg

SEQ ID NO: 374 - DNA, 20 bp, synthetic construct
  misc_feature 1..20: note = Synthetic
  source 1..20: mol_type = other DNA
gctgcagctcagctgactgt

SEQ ID NO: 375 - DNA, 20 bp, synthetic construct
  misc_feature 1..20: note = Synthetic
  source 1..20: mol_type = other DNA
cgctgcagctcagctgactg

SEQ ID NO: 376 - DNA, 20 bp, synthetic construct
  misc_feature 1..20: note = Synthetic
  source 1..20: mol_type = other DNA
gcgctgcagctcagctgact

SEQ ID NO: 377 - DNA, 20 bp, synthetic construct
  misc_feature 1..20: note = Synthetic
  source 1..20: mol_type = other DNA
agcgctgcagctcagctgac

SEQ ID NO: 378 - DNA, 20 bp, synthetic construct
  misc_feature 1..20: note = Synthetic
  source 1..20: mol_type = other DNA
aagcgctgcagctcagctga

SEQ ID NO: 379 - DNA, 20 bp, synthetic construct
  misc_feature 1..20: note = Synthetic
  source 1..20: mol_type = other DNA
caagcgctgcagctcagctg

SEQ ID NO: 380 - DNA, 20 bp, synthetic construct
  misc_feature 1..20: note = Synthetic
  source 1..20: mol_type = other DNA
ccaagcgctgcagctcagct

SEQ ID NO: 381 - DNA, 20 bp, synthetic construct
  misc_feature 1..20: note = Synthetic
  source 1..20: mol_type = other DNA
gccaagcgctgcagctcagc

SEQ ID NO: 382 - DNA, 20 bp, synthetic construct
  misc_feature 1..20: note = Synthetic
  source 1..20: mol_type = other DNA
cgccaagcgctgcagctcag

SEQ ID NO: 383 - DNA, 20 bp, synthetic construct
  misc_feature 1..20: note = Synthetic
  source 1..20: mol_type = other DNA
gcgccaagcgctgcagctca

SEQ ID NO: 384 - DNA, 20 bp, synthetic construct
  misc_feature 1..20: note = Synthetic
  source 1..20: mol_type = other DNA
agcgccaagcgctgcagctc

SEQ ID NO: 385 - DNA, 20 bp, synthetic construct
  misc_feature 1..20: note = Synthetic
  source 1..20: mol_type = other DNA
ccagcgccaagcgctgcagc

SEQ ID NO: 386 - DNA, 20 bp, synthetic construct
  misc_feature 1..20: note = Synthetic
  source 1..20: mol_type = other DNA
tccagcgccaagcgctgcag

SEQ ID NO: 387 - DNA, 20 bp, synthetic construct
  misc_feature 1..20: note = Synthetic
  source 1..20: mol_type = other DNA
ttccagcgccaagcgctgca

SEQ ID NO: 388 - DNA, 20 bp, synthetic construct
  misc_feature 1..20: note = Synthetic
  source 1..20: mol_type = other DNA
tgttccagcgccaagcgctg

SEQ ID NO: 389 - DNA, 20 bp, synthetic construct
  misc_feature 1..20: note = Synthetic
  source 1..20: mol_type = other DNA
ctgttccagcgccaagcgct

SEQ ID NO: 390 - DNA, 20 bp, synthetic construct
  misc_feature 1..20: note = Synthetic
  source 1..20: mol_type = other DNA
gctgttccagcgccaagcgc

SEQ ID NO: 391 - DNA, 20 bp, synthetic construct
  misc_feature 1..20: note = Synthetic
  source 1..20: mol_type = other DNA
ctgctgttccagcgccaagc

SEQ ID NO: 392 - DNA, 20 bp, synthetic construct
  misc_feature 1..20: note = Synthetic
  source 1..20: mol_type = other DNA
gctgctgttccagcgccaag

SEQ ID NO: 393 - DNA, 20 bp, synthetic construct
  misc_feature 1..20: note = Synthetic
  source 1..20: mol_type = other DNA
tgctgctgttccagcgccaa

SEQ ID NO: 394 - DNA, 20 bp, synthetic construct
  misc_feature 1..20: note = Synthetic
  source 1..20: mol_type = other DNA
ctgctgctgttccagcgcca

SEQ ID NO: 395 - DNA, 20 bp, synthetic construct
  misc_feature 1..20: note = Synthetic
  source 1..20: mol_type = other DNA
actgctgctgttccagcgcc

SEQ ID NO: 396 - DNA, 20 bp, synthetic construct
  misc_feature 1..20: note = Synthetic
  source 1..20: mol_type = other DNA
cactgctgctgttccagcgc

SEQ ID NO: 397 - DNA, 20 bp, synthetic construct
  misc_feature 1..20: note = Synthetic
  source 1..20: mol_type = other DNA
ccactgctgctgttccagcg

SEQ ID NO: 398 - DNA, 20 bp, synthetic construct
  misc_feature 1..20: note = Synthetic
  source 1..20: mol_type = other DNA
gccactgctgctgttccagc

SEQ ID NO: 399 - DNA, 20 bp, synthetic construct
  misc_feature 1..20: note = Synthetic
  source 1..20: mol_type = other DNA
agccactgctgctgttccag

SEQ ID NO: 400 - DNA, 20 bp, synthetic construct
  misc_feature 1..20: note = Synthetic
  source 1..20: mol_type = other DNA
cagccactgctgctgttcca

SEQ ID NO: 401 - DNA, 20 bp, synthetic construct
  misc_feature 1..20: note = Synthetic
  source 1..20: mol_type = other DNA
gcagccactgctgctgttcc

SEQ ID NO: 402 - DNA, 20 bp, synthetic construct
  misc_feature 1..20: note = Synthetic
  source 1..20: mol_type = other DNA
tgcagccactgctgctgttc

SEQ ID NO: 403 - DNA, 20 bp, synthetic construct
  misc_feature 1..20: note = Synthetic
  source 1..20: mol_type = other DNA
atgcagccactgctgctgtt

SEQ ID NO: 404 - DNA, 20 bp, synthetic construct
  misc_feature 1..20: note = Synthetic
  source 1..20: mol_type = other DNA
ggcagcggcacactgtgcag

SEQ ID NO: 405 - DNA, 20 bp, synthetic construct
  misc_feature 1..20: note = Synthetic
  source 1..20: mol_type = other DNA
aggcagcggcacactgtgca

SEQ ID NO: 406 - DNA, 20 bp, synthetic construct
  misc_feature 1..20: note = Synthetic
  source 1..20: mol_type = other DNA
caggcagcggcacactgtgc

SEQ ID NO: 407 - DNA, 20 bp, synthetic construct
  misc_feature 1..20: note = Synthetic
  source 1..20: mol_type = other DNA
gcaggcagcggcacactgtg

SEQ ID NO: 408 - DNA, 20 bp, synthetic construct
  misc_feature 1..20: note = Synthetic
  source 1..20: mol_type = other DNA
ggcaggcagcggcacactgt

SEQ ID NO: 409 - DNA, 20 bp, synthetic construct
  misc_feature 1..20: note = Synthetic
  source 1..20: mol_type = other DNA
ttggccttctgtctgtccct

SEQ ID NO: 410 - DNA, 20 bp, synthetic construct
  misc_feature 1..20: note = Synthetic
  source 1..20: mol_type = other DNA
cttggccttctgtctgtccc

SEQ ID NO: 411 - DNA, 20 bp, synthetic construct
  misc_feature 1..20: note = Synthetic
  source 1..20: mol_type = other DNA
ccttggccttctgtctgtcc

SEQ ID NO: 412 - DNA, 20 bp, synthetic construct
  misc_feature 1..20: note = Synthetic
  source 1..20: mol_type = other DNA
gccttggccttctgtctgtc

SEQ ID NO: 413 - DNA, 20 bp, synthetic construct
  misc_feature 1..20: note = Synthetic
  source 1..20: mol_type = other DNA
ggccttggccttctgtctgt

SEQ ID NO: 414 - DNA, 20 bp, synthetic construct
  misc_feature 1..20: note = Synthetic
  source 1..20: mol_type = other DNA
gggccttggccttctgtctg

SEQ ID NO: 415 - DNA, 20 bp, synthetic construct
  misc_feature 1..20: note = Synthetic
  source 1..20: mol_type = other DNA
cgggccttggccttctgtct

SEQ ID NO: 416 - DNA, 20 bp, synthetic construct
  misc_feature 1..20: note = Synthetic
  source 1..20: mol_type = other DNA
tcgggccttggccttctgtc

SEQ ID NO: 417 - DNA, 20 bp, synthetic construct
  misc_feature 1..20: note = Synthetic
  source 1..20: mol_type = other DNA
atcgggccttggccttctgt

SEQ ID NO: 418 - DNA, 20 bp, synthetic construct
  misc_feature 1..20: note = Synthetic
  source 1..20: mol_type = other DNA
catcgggccttggccttctg

SEQ ID NO: 419 - DNA, 20 bp, synthetic construct
  misc_feature 1..20: note = Synthetic
  source 1..20: mol_type = other DNA
acatcgggccttggccttct

SEQ ID NO: 420 - DNA, 20 bp, synthetic construct
  misc_feature 1..20: note = Synthetic
  source 1..20: mol_type = other DNA
cacatcgggccttggccttc

SEQ ID NO: 421 - DNA, 20 bp, synthetic construct
  misc_feature 1..20: note = Synthetic
  source 1..20: mol_type = other DNA
ccacatcgggccttggcctt

SEQ ID NO: 422 - DNA, 20 bp, synthetic construct
  misc_feature 1..20: note = Synthetic
  source 1..20: mol_type = other DNA
accacatcgggccttggcct

SEQ ID NO: 423 - DNA, 20 bp, synthetic construct
  misc_feature 1..20: note = Synthetic
  source 1..20: mol_type = other DNA
caccacatcgggccttggcc

SEQ ID NO: 424 - DNA, 20 bp, synthetic construct
  misc_feature 1..20: note = Synthetic
  source 1..20: mol_type = other DNA
cacaccacatcgggccttgg

SEQ ID NO: 425 - DNA, 20 bp, synthetic construct
  misc_feature 1..20: note = Synthetic
  source 1..20: mol_type = other DNA
gcacaccacatcgggccttg

SEQ ID NO: 426 - DNA, 20 bp, synthetic construct
  misc_feature 1..20: note = Synthetic
  source 1..20: mol_type = other DNA
tgcacaccacatcgggcctt

SEQ ID NO: 427 - DNA, 20 bp, synthetic construct
  misc_feature 1..20: note = Synthetic
  source 1..20: mol_type = other DNA
tctgcacaccacatcgggcc

SEQ ID NO: 428 - DNA, 20 bp, synthetic construct
  misc_feature 1..20: note = Synthetic
  source 1..20: mol_type = other DNA
ctctgcacaccacatcgggc

SEQ ID NO: 429 - DNA, 20 bp, synthetic construct
  misc_feature 1..20: note = Synthetic
  source 1..20: mol_type = other DNA
cctctgcacaccacatcggg

SEQ ID NO: 430 - DNA, 20 bp, synthetic construct
  misc_feature 1..20: note = Synthetic
  source 1..20: mol_type = other DNA
cacctctgcacaccacatcg

SEQ ID NO: 431 - DNA, 20 bp, synthetic construct
  misc_feature 1..20: note = Synthetic
  source 1..20: mol_type = other DNA
ccacctctgcacaccacatc

SEQ ID NO: 432 - DNA, 20 bp, synthetic construct
  misc_feature 1..20: note = Synthetic
  source 1..20: mol_type = other DNA
cccacctctgcacaccacat

SEQ ID NO: 433 - DNA, 20 bp, synthetic construct
  misc_feature 1..20: note = Synthetic
  source 1..20: mol_type = other DNA
ccccacctctgcacaccaca

SEQ ID NO: 434 - DNA, 20 bp, synthetic construct
  misc_feature 1..20: note = Synthetic
  source 1..20: mol_type = other DNA
tccccacctctgcacaccac

SEQ ID NO: 435 - DNA, 20 bp, synthetic construct
  misc_feature 1..20: note = Synthetic
  source 1..20: mol_type = other DNA
ctccccacctctgcacacca

SEQ ID NO: 436 - DNA, 20 bp, synthetic construct
  misc_feature 1..20: note = Synthetic
  source 1..20: mol_type = other DNA
cctccccacctctgcacacc

SEQ ID NO: 437 - DNA, 20 bp, synthetic construct
  misc_feature 1..20: note = Synthetic
  source 1..20: mol_type = other DNA
acctccccacctctgcacac

SEQ ID NO: 438 - DNA, 20 bp, synthetic construct
  misc_feature 1..20: note = Synthetic
  source 1..20: mol_type = other DNA
cacctccccacctctgcaca

SEQ ID NO: 439 - DNA, 20 bp, synthetic construct
  misc_feature 1..20: note = Synthetic
  source 1..20: mol_type = other DNA
ccacctccccacctctgcac

SEQ ID NO: 440 - DNA, 20 bp, synthetic construct
  misc_feature 1..20: note = Synthetic
  source 1..20: mol_type = other DNA
aggggcaggggtggcacacg

SEQ ID NO: 441 - DNA, 20 bp, synthetic construct
  misc_feature 1..20: note = Synthetic
  source 1..20: mol_type = other DNA
gaggggcaggggtggcacac

SEQ ID NO: 442 - DNA, 20 bp, synthetic construct
  misc_feature 1..20: note = Synthetic
  source 1..20: mol_type = other DNA
ggaggggcaggggtggcaca

SEQ ID NO: 443 - DNA, 20 bp, synthetic construct
  misc_feature 1..20: note = Synthetic
  source 1..20: mol_type = other DNA
gggaggggcaggggtggcac

SEQ ID NO: 444 - DNA, 20 bp, synthetic construct
  misc_feature 1..20: note = Synthetic
  source 1..20: mol_type = other DNA
ggggaggggcaggggtggca

SEQ ID NO: 445 - DNA, 20 bp, synthetic construct
  misc_feature 1..20: note = Synthetic
  source 1..20: mol_type = other DNA
cggggaggggcaggggtggc

SEQ ID NO: 446 - DNA, 20 bp, synthetic construct
  misc_feature 1..20: note = Synthetic
  source 1..20: mol_type = other DNA
tcggggaggggcaggggtgg

SEQ ID NO: 447 - DNA, 20 bp, synthetic construct
  misc_feature 1..20: note = Synthetic
  source 1..20: mol_type = other DNA
atcggggaggggcaggggtg

SEQ ID NO: 448 - DNA, 20 bp, synthetic construct
  misc_feature 1..20: note = Synthetic
  source 1..20: mol_type = other DNA
gatcggggaggggcaggggt

SEQ ID NO: 449 - DNA, 20 bp, synthetic construct
  misc_feature 1..20: note = Synthetic
  source 1..20: mol_type = other DNA
ggatcggggaggggcagggg

SEQ ID NO: 450 - DNA, 20 bp, synthetic construct
  misc_feature 1..20: note = Synthetic
  source 1..20: mol_type = other DNA
gggatcggggaggggcaggg

SEQ ID NO: 451 - DNA, 20 bp, synthetic construct
  misc_feature 1..20: note = Synthetic
  source 1..20: mol_type = other DNA
agggatcggggaggggcagg

SEQ ID NO: 452 - DNA, 20 bp, synthetic construct
  misc_feature 1..20: note = Synthetic
  source 1..20: mol_type = other DNA
cagggatcggggaggggcag

SEQ ID NO: 453 - DNA, 20 bp, synthetic construct
  misc_feature 1..20: note = Synthetic
  source 1..20: mol_type = other DNA
acagggatcggggaggggca

SEQ ID NO: 454 - DNA, 20 bp, synthetic construct
  misc_feature 1..20: note = Synthetic
  source 1..20: mol_type = other DNA
cacagggatcggggaggggc

SEQ ID NO: 455 - DNA, 20 bp, synthetic construct
  misc_feature 1..20: note = Synthetic
  source 1..20: mol_type = other DNA
acacagggatcggggagggg

SEQ ID NO: 456 - DNA, 20 bp, synthetic construct
  misc_feature 1..20: note = Synthetic
  source 1..20: mol_type = other DNA
cacacagggatcggggaggg

SEQ ID NO: 457 - DNA, 20 bp, synthetic construct
  misc_feature 1..20: note = Synthetic
  source 1..20: mol_type = other DNA
gcacacagggatcggggagg

SEQ ID NO: 458 - DNA, 20 bp, synthetic construct
  misc_feature 1..20: note = Synthetic
  source 1..20: mol_type = other DNA
cgcacacagggatcggggag

SEQ ID NO: 459 - DNA, 20 bp, synthetic construct
  misc_feature 1..20: note = Synthetic
  source 1..20: mol_type = other DNA
gcgcacacagggatcgggga

SEQ ID NO: 460 - DNA, 20 bp, synthetic construct
  misc_feature 1..20: note = Synthetic
  source 1..20: mol_type = other DNA
cgcgcacacagggatcgggg

SEQ ID NO: 461 - DNA, 20 bp, synthetic construct
  misc_feature 1..20: note = Synthetic
  source 1..20: mol_type = other DNA
gcgcgcacacagggatcggg

SEQ ID NO: 462 - DNA, 20 bp, synthetic construct
  misc_feature 1..20: note = Synthetic
  source 1..20: mol_type = other DNA
ggcgcgcacacagggatcgg

SEQ ID NO: 463 - DNA, 20 bp, synthetic construct
  misc_feature 1..20: note = Synthetic
  source 1..20: mol_type = other DNA
ggggcgcgcacacagggatc

SEQ ID NO: 464 - DNA, 20 bp, synthetic construct
  misc_feature 1..20: note = Synthetic
  source 1..20: mol_type = other DNA
gggggcgcgcacacagggat

SEQ ID NO: 465 - DNA, 20 bp, synthetic construct
  misc_feature 1..20: note = Synthetic
  source 1..20: mol_type = other DNA
tcaacaagcagggtcggagt

SEQ ID NO: 466 - DNA, 20 bp, synthetic construct
  misc_feature 1..20: note = Synthetic
  source 1..20: mol_type = other DNA
ttgaacgcagcgtgaagttg

SEQ ID NO: 467 - DNA, 21 bp, synthetic construct
  misc_feature 1..21: note = Synthetic
  source 1..21: mol_type = other DNA
cttcggttgtcctggtcgata

SEQ ID NO: 468 - DNA, 20 bp, synthetic construct
  misc_feature 1..20: note = Synthetic
  source 1..20: mol_type = other DNA
ttgcgattgccaggagacat

SEQ ID NO: 469 - DNA, 22 bp, synthetic construct
  misc_feature 1..22: note = Synthetic
  source 1..22: mol_type = other DNA
gaagatcaagatcattgctcct

SEQ ID NO: 470 - DNA, 20 bp, synthetic construct
  misc_feature 1..20: note = Synthetic
  source 1..20: mol_type = other DNA
tactcctgcttgctgatcca

SEQ ID NO: 471 - DNA, 4355 bp, Homo sapiens
  source 1..4355: mol_type = genomic DNA
gcaggacgccatactggacgccaagtgggaggaacttcaaggctgtcccctgcgggcctcccgctctgcttctgcgaaggtttcattgaaaacagatcctgcaaaagttccaggtgcccacactggaaacttggagatcctgcttcccagaccacagctgtggggaacttggggtggagcagagaagtttctgtattcagctgcccaggcagaggagaatggggtctccacagcctgaagaatgaagacacgacagaataaagactcgatgtcaatgaggagtggacggaagaaagaggcccctgggccccgggaagaactgagatcgaggggccgggcctcccctggaggggtcagcacgtccagcagtgatggcaaagctgagaagtccaggcagacagccaagaaggcccgagtagaggaagcctccaccccaaaggtcaacaagcagggtcggagtgaggagatctcagagagtgaaagtgaggagaccaatgcaccaaaaaagaccaaaactgagcaggaactccctcggccacagtctccctccgatctggatagcttggacgggcggagccttaatgatgatggcagcagcgaccctagggatatcgaccaggacaaccgaagcacgtcccccagtatctacagccctggaagtgtggagaatgactctgactcatcttctggcctgtcccagggcccagcccgcccctaccacccacctccactctttcctccttcccctcaaccgccagacagcacccctcgacagccagaggctagctttgaaccccatccttctgtgacacccactggatatcatgctcccatggagccccccacatctcgaatgttccaggctcctcctggggcccctccccctcacccacagctctatcctgggggcactggtggagttttgtctggacccccaatgggtcccaaggggggaggggctgcctcatcagtggggggccctaatgggggtaagcagcaccccccacccactactcccatttcagtatcaagctctggggctagtggtgctcccccaacaaagccgcctaccactccagtgggtggtgggaacctaccttctgctccaccaccagccaacttcccccatgtgacaccgaacctgcctcccccacctgccctgagacccctcaacaatgcatcagcctctccccctggcctgggggcccaaccactacctggtcatctgccctctccccacgccatgggacagggtatgggtggacttcctcctggcccagagaagggcccaactctggctccttcaccccactctctgcctcctgcttcctcttctgctccagcgccccccatgaggtttccttattcatcctctagtagtagctctgcagcagcctcctcttccagttcttcctcctcttcctctgcctcccccttcccagcttcccaggcattgcccagctacccccactctttccctcccccaacaagcctctctgtctccaatcagccccccaagtatactcagccttctctcccatcccaggctgtgtggagccagggtcccccaccacctcctccctatggccgcctcttagccaacagcaatgcccatccaggccccttccctccctctactggggcccagtccaccgcccacccaccagtctcaacacatcaccatcaccaccagcaacagcaacagcagcagcagcagcagcagcagcagcagcagcagcagcagcagcatcacggaaactctgggccccctcctcctggagcatttccccacccactggagggcggtagctcccaccacgcacacccttacgccatgtctccctccctggggtctctgaggccctacccaccagggccagcacacctgcccccacctcacagccaggtgtcctacagccaagcaggccccaatggccctccagtctcttcctcttccaactcttcctcttccacttctcaagggtcctacccatgttcacacccctccccttcccagggccctcaaggggcgccctaccctttcccaccggtgcctacggtcaccacctcttcggctaccctttccacggtcattgccaccgtggcttcctcgccagcaggctacaaaacggcctccccacctgggcccccaccgtacggaaagagagccccgtccccgggggcctacaagacagccaccccacccggatacaaacccgggtcgcctccctccttccgaacggggaccccaccgggctatcgaggaacctcgccacctgcaggcccagggaccttcaagccgggctcgcccaccgtgggacctgggcccctgccacctgcggggccctcaggcctgccatcgctgccaccaccacctgcggcccctgcctcagggccgcccctgagcgccacgcagatcaaacaggagccggctgaggagtatgagacccccgagagcccggtgcccccagcccgcagcccctcgccccctcccaaggtggtagatgtacccagccatgccagtcagtctgccaggttcaacaaacacctggatcgcggcttcaactcgtgcgcgcgcagcgacctgtacttcgtgccactggagggctccaagctggccaagaagcgggccgacctggtggagaaggtgcggcgcgaggccgagcagcgcgcgcgcgaagaaaaggagcgcgagcgcgagcgggaacgcgagaaagagcgcgagcgcgagaaggagcgcgagcttgaacgcagcgtgaagttggctcaggagggccgtgctccggtggaatgcccatctctgggcccagtgccccatcgccctccatttgaaccgggcagtgcggtggctacagtgcccccctacctgggtcctgacactccagccttgcgcactctcagtgaatatgcccggcctcatgtcatgtctcctggcaatcgcaaccatccattctacgtgcccctgggggcagtggacccggggctcctgggttacaatgtcccggccctgtacagcagtgatccagctgcccgggagagggaacgggaagcccgtgaacgagacctccgtgaccgcctcaagcctggctttgaggtgaagcctagtgagctggaacccctacatggggtccctgggccgggcttggatccctttccccgacatgggggcctggctctgcagcctggcccacctggcctgcaccctttcccctttcatccgagcctggggcccctggagcgagaacgtctagcgctggcagctgggccagccctgcggcctgacatgtcctatgctgagcggctggcagctgagaggcagcacgcagaaagggtggcggccctgggcaatgacccactggcccggctgcagatgctcaatgtgactccccatcaccaccagcactcccacatccactcgcacctgcacctgcaccagcaagatgctatccatgcagcctctgcctcggtgcaccctctcattgaccccctggcctcagggtctcaccttacccggatcccctacccagctggaactctccctaaccccctgcttcctcaccctctgcacgagaacgaagttcttcgtcaccagctctttgctgccccttaccgggacctgccggcctccctttctgccccgatgtcagcagctcatcagctgcaggccatgcacgcacagtcagctgagctgcagcgcttggcgctggaacagcagcagtggctgcatgcccatcacccgctgcacagtgtgccgctgcctgcccaggaggactactacagtcacctgaagaaggaaagcgacaagccactgtagaacctgcgatcaagagagcaccatggctcctacattggaccttggagcacccccaccctccccccaccgtgcccttggcctgccacccagagccaagagggtgctgctcagttgcagggcctccgcagctggacagagagtgggggagggagggacagacagaaggccaaggcccgatgtggtgtgcagaggtggggaggtggcgaggatggggacagaaagcgcacagaatcttggaccaggtctctcttccttgtcccccctgcttttctcctcccccatgcccaacccctgtggccgccgcccctcccctgccccgttggtgtgattatttcatctgttagatgtggctgttttgcgtagcatcgtgtgccacccctgcccctccccgatccctgtgtgcgcgccccctctgcaatgtatgccccttgccccttccccacactaataatttatatatataaatatctatatgacgctcttaaaaaaacatcccaaccaaaaccaaccaaacaaaaacatcctcacaactccccaggaa

SEQ ID NO: 472 - DNA, 20 bp, synthetic construct
  misc_feature 1..20: note = Synthetic
  source 1..20: mol_type = other DNA
agagacctggtccaagattc

SEQ ID NO: 473 - DNA, 20 bp, synthetic construct
  source 1..20: mol_type = other DNA
ctcgcgctcgcgctccttct

(19) United States
(12) Patent Application Publication
(10) Pub. No.: US 2025/0051769 A1
(43) Pub. Date: Feb. 13, 2025

(54) ANTISENSE OLIGONUCLEOTIDE TARGETING ATN1 MRNA OR PRE-MRNA

(71) Applicants: NIPPON SHINYAKU CO., LTD., Minami-ku, Kyoto-shi, Kyoto (JP); NIIGATA UNIVERSITY, Niigata-shi, Niigata (JP)

(72) Inventors: Takao KAWANO, Kyoto (JP); Toshiaki ODE, Kyoto (JP); Akie CHIBA, Ibaraki (JP); Osamu ONODERA, Niigata (JP); Taisuke KATO, Niigata (JP); Sachiko HIROKAWA, Niigata (JP)

(73) Assignees: NIPPON SHINYAKU CO., LTD., Minami-ku, Kyoto-shi, Kyoto (JP); NIIGATA UNIVERSITY, Niigata-shi, Niigata (JP)

(21) Appl. No.: 18/719,037

(22) PCT Filed: Dec. 13, 2022
(86) PCT No.: PCT/JP2022/045921

§ 371 Date: Jun. 12, 2024

(30) Foreign Application Priority Data
Dec. 13, 2021 (JP) 2021-201756
Nov. 18, 2022 (JP) 2022-184853

(51) Int. Cl. C12N 15/113

(52) U.S. Cl. CPC C12N 15/113; C12N 2310/11; C12N 2310/315; C12N 2310/321; C12N 2310/341

(57) ABSTRACT

The present invention provides an antisense oligonucleotide that is formed of 15-22 nucleotides and that is complementary to a nucleic acid including at least 15 consecutive bases in a specific target region of a base sequence of SEQ ID NO: 471, or a pharmaceutically acceptable salt thereof, or hydrates of those.

### TECHNICAL FIELD

The present invention relates to an antisense oligonucleotide targeting an ATN1 mRNA or pre-mRNA, or a pharmaceutically acceptable salt thereof, or a hydrate thereof, and a pharmaceutical composition or the like comprising the antisense oligonucleotide or a pharmaceutically acceptable salt thereof, or a hydrate thereof.

### BACKGROUND ART

Dentatorubral-pallidoluysian atrophy (DRPLA) is a progressive disease that causes ataxia, involuntary movement designated as myoclonus, epilepsy, progressive intellectual deterioration and the like in a child patient, and causes ataxia, choreoathetosis, dementia, personality change or the like in an adult patient (Non Patent Literature 1).

DRPLA is a disease (repeat disease) caused due to RNA or protein functional abnormality caused by abnormal elongation of a repeat sequence present in the genome of a specific gene. DRPLA is known, among repeat diseases, as a disease in which a protein exhibits toxicity. DRPLA is divided into adult-onset type that develops at the age of 20 or older, and childhood-onset type that develops at the age younger than 20 depending on the time of onset, and early developing childhood-onset type is caused and the degree of seriousness is high in a patient having the number of repeats of about 65 or more.

Currently, although there are some symptomatic drugs for some of the symptoms of DRPLA, that is, epilepsy, a psychiatric symptom, ataxia, and the like, treatment satisfaction of these is low, and it is impossible to cope with molecular mechanism underlying DRPLA.

### CITATION LIST

### Non Patent Literature

Non Patent Literature 1: Liana Veneziano et al., Gene Reviews [Internet]1999 Aug. 6 [updated 2016 Jun. 9], 1993-2021

### SUMMARY OF INVENTION

Under these circumstances, it is desired to provide a DRPLA therapeutic agent exhibiting more excellent therapeutic effects.

The present invention provides an antisense oligonucleotide targeting an ATN1 mRNA or pre-mRNA, or a pharmaceutically acceptable salt thereof, or a hydrate thereof, and a pharmaceutical composition or the like comprising the antisense oligonucleotide, or a pharmaceutically acceptable salt thereof, or a hydrate thereof as follows.

(1-1) An antisense oligonucleotide consisting of 15 to 22 nucleotides complementary to a nucleic acid comprising at least 15 consecutive bases in a target region selected from the group consisting of positions 1 to 84, 142 to 168, 197 to 224, 286 to 317, 324 to 370, 391 to 434, 519 to 540, 623 to 643, 690 to 780, 824 to 855, 860 to 897, 948 to 987, 1044 to 1072, 1125 to 1174, 1181 to 1213, 1228 to 1255, 1265 to 1327, 1334 to 1356, 1416 to 1440, 1447 to 1631, 1638 to 1667, 1675 to 1705, 1748 to 1823, 1838 to 1861, 1870 to 1913, 1920 to 1941, 2000 to 2040, 2047 to 2075, 2086 to 2120, 2129 to 2187, 2194 to 2415, 2451 to 2497, 2592 to 2759, 2766 to 2870, 2928 to 2948, 2955 to 2989, 3021 to 3086, 3133 to 3209, 3217 to 3284, 3295 to 3350, 3384 to 3436, 3562 to 3771, 3858 to 3905, 3931 to 4039, 4067 to 4134, 4170 to 4232, 4241 to 4283, and 4286 to 4355 of a base sequence of SEQ ID NO: 471, or a pharmaceutically acceptable salt thereof, or a hydrate thereof.

(1-2) The antisense oligonucleotide, or a pharmaceutically acceptable salt thereof, or a hydrate thereof according to (1-1), wherein the antisense oligonucleotide is complementary to a nucleic acid comprising at least 15 consecutive bases in a target region selected from the group consisting of positions 1 to 84, 142 to 168, 391 to 434, 697 to 780, 962 to 980, 1125 to 1174, 1181 to 1213, 1228 to 1255, 1265 to 1320, 1454 to 1631, 1682 to 1705, 1748 to 1816, 1870 to 1913, 2015 to 2040, 2086 to 2120, 2129 to 2187, 2201 to 2408, 2592 to 2759, 2773 to 2870, 2955 to 2982, 3028 to 3079, 3133 to 3209, 3224 to 3277, 3295 to 3350, 3391 to 3429, 3562 to 3771, 3865 to 3898, 3938 to 4032, 4074 to 4127, and 4170 to 4225 of the base sequence of SEQ ID NO: 471.

(2-1) The antisense oligonucleotide or a pharmaceutically acceptable salt thereof, or a hydrate thereof according to (1-1), wherein the antisense oligonucleotide is complementary to a nucleic acid comprising at least 15 consecutive bases in a target region selected from the group consisting of positions 1 to 69, 331 to 363, 398 to 427, 697 to 773, 867 to 890, 955 to 980, 1132 to 1167, 1272 to 1320, 1454 to 1624, 1682 to 1705, 1748 to 1816, 1877 to 1906, 2007 to 2033, 2093 to 2113, 2136 to 2180, 2201 to 2408, 2458 to 2490, 2599 to 2752, 2773 to 2863, 2962 to 2982, 3028 to 3079, 3140 to 3202, 3224 to 3277, 3302 to 3343, 3391 to 3429, 3569 to 3764, 3865 to 3898, 3938 to 4032, 4074 to 4127, 4177 to 4225, 4248 to 4276, and 4293 to 4355 of the base sequence of SEQ ID NO: 471.

(2-2) The antisense oligonucleotide, or a pharmaceutically acceptable salt thereof, or a hydrate thereof according to (1-1), wherein the antisense oligonucleotide is complementary to a nucleic acid comprising at least 15 consecutive bases in a target region selected from the group consisting of positions 1 to 69, 398 to 427, 697 to 773, 1132 to 1167, 1272 to 1320, 1454 to 1624, 1682 to 1705, 1748 to 1816, 1877 to 1906, 2093 to 2113, 2136 to 2180, 2201 to 2408, 2599 to 2752, 2773 to 2863, 2962 to 2982, 3028 to 3079, 3140 to 3202, 3224 to 3277, 3302 to 3343, 3391 to 3429, 3569 to 3764, 3865 to 3898, 3938 to 4032, and 4177 to 4225 of the base sequence of SEQ ID NO: 471.

(3) The antisense oligonucleotide or a pharmaceutically acceptable salt thereof, or a hydrate thereof according to (1-1), wherein the antisense oligonucleotide is complementary to a nucleic acid comprising at least 15 consecutive bases in a target region selected from the group consisting of positions 14 to 33, 34 to 53, 54 to 73, 148 to 167, 397 to 432, 723 to 742, 747 to 778, 965 to 984, 1127 to 1146, 1148 to 1173, 1182 to 1213, 1228 to 1254, 1270 to 1308, 1484 to 1564, 1578 to 1629, 1686 to 1705, 1748 to 1767, 1768 to 1787, 1794 to 1813, 1870 to 1913, 2015 to 2034, 2086 to 2118, 2129 to 2148, 2151 to 2184, 2206 to 2225, 2239 to 2272, 2287 to 2336, 2337 to 2373, 2387 to 2406, 2598 to 2757, 2775 to 2809, 2826 to 2866, 2959 to 2982, 3032 to 3051, 3052 to 3071, 3136 to 3155, 3168 to 3187, 3188 to 3207, 3232 to 3270, 3298 to 3344, 3408 to 3427, 3563 to 3582, 3590 to 3728, 3739 to 3769, 3877 to 3896, 3949 to 4030, and 4170 to 4225 of the base sequence of SEQ ID NO: 471.

(4) The antisense oligonucleotide or a pharmaceutically acceptable salt thereof, or a hydrate thereof according to (3), wherein the antisense oligonucleotide is complementary to a nucleic acid comprising at least 15 consecutive bases in a target region selected from the group consisting of positions 14 to 33, 34 to 53, 54 to 73, 148 to 167, 397 to 416, 398 to 417, 399 to 418, 400 to 419, 401 to 420, 402 to 421, 403 to 422, 404 to 423, 405 to 424, 406 to 425, 407 to 426, 408 to 427, 413 to 432, 723 to 742, 747 to 766, 756 to 775, 759 to 778, 965 to 984, 1127 to 1146, 1148 to 1167, 1151 to 1170, 1154 to 1173, 1182 to 1201, 1185 to 1204, 1188 to 1207, 1191 to 1210, 1194 to 1213, 1228 to 1247, 1231 to 1250, 1235 to 1254, 1270 to 1289, 1289 to 1308, 1484 to 1503, 1488 to 1507, 1491 to 1510, 1493 to 1512, 1506 to 1525, 1517 to 1536, 1531 to 1550, 1545 to 1564, 1578 to 1597, 1579 to 1598, 1580 to 1599, 1581 to 1600, 1582 to 1601, 1583 to 1602, 1584 to 1603, 1585 to 1604, 1586 to 1605, 1587 to 1606, 1588 to 1607, 1589 to 1608, 1590 to 1609, 1591 to 1610, 1592 to 1611, 1593 to 1612, 1594 to 1613, 1595 to 1614, 1596 to 1615, 1597 to 1616, 1598 to 1617, 1599 to 1618, 1600 to 1619, 1601 to 1620, 1602 to 1621, 1603 to 1622, 1604 to 1623, 1605 to 1624, 1607 to 1626, 1610 to 1629, 1686 to 1705, 1748 to 1767, 1768 to 1787, 1794 to 1813, 1870 to 1889, 1871 to 1890, 1874 to 1893, 1877 to 1896, 1878 to 1897, 1879 to 1898, 1880 to 1899, 1881 to 1900, 1882 to 1901, 1883 to 1902, 1884 to 1903, 1885 to 1904, 1886 to 1905, 1887 to 1906, 1889 to 1908, 1894 to 1913, 2015 to 2034, 2086 to 2105, 2093 to 2112, 2094 to 2113, 2097 to 2116, 2099 to 2118, 2129 to 2148, 2151 to 2170, 2165 to 2184, 2206 to 2225, 2239 to 2258, 2253 to 2272, 2287 to 2306, 2301 to 2320, 2317 to 2336, 2337 to 2356, 2354 to 2373, 2387 to 2406, 2598 to 2617, 2613 to 2632, 2614 to 2633, 2615 to 2634, 2616 to 2635, 2617 to 2636, 2618 to 2637, 2619 to 2638, 2620 to 2639, 2621 to 2640, 2622 to 2641, 2623 to 2642, 2624 to 2643, 2625 to 2644, 2626 to 2645, 2627 to 2646, 2628 to 2647, 2629 to 2648, 2630 to 2649, 2631 to 2650, 2632 to 2651, 2633 to 2652, 2634 to 2653, 2635 to 2654, 2636 to 2655, 2637 to 2656, 2638 to 2657, 2639 to 2658, 2640 to 2659, 2641 to 2660, 2642 to 2661, 2643 to 2662, 2644 to 2663, 2645 to 2664, 2646 to 2665, 2647 to 2666, 2648 to 2667, 2649 to 2668, 2663 to 2682, 2672 to 2691, 2673 to 2692, 2674 to 2693, 2675 to 2694, 2676 to 2695, 2677 to 2696, 2678 to 2697, 2679 to 2698, 2680 to 2699, 2681 to 2700, 2682 to 2701, 2683 to 2702, 2684 to 2703, 2685 to 2704, 2686 to 2705, 2687 to 2706, 2688 to 2707, 2689 to 2708, 2690 to 2709, 2691 to 2710, 2692 to 2711, 2693 to 2712, 2694 to 2713, 2695 to 2714, 2696 to 2715, 2697 to 2716, 2698 to 2717, 2699 to 2718, 2700 to 2719, 2701 to 2720, 2702 to 2721, 2703 to 2722, 2704 to 2723, 2705 to 2724, 2706 to 2725, 2707 to 2726, 2708 to 2727, 2709 to 2728, 2710 to 2729, 2711 to 2730, 2712 to 2731, 2713 to 2732, 2714 to 2733, 2715 to 2734, 2716 to 2735, 2717 to 2736, 2718 to 2737, 2719 to 2738, 2720 to 2739, 2721 to 2740, 2722 to 2741, 2723 to 2742, 2724 to 2743, 2725 to 2744, 2726 to 2745, 2727 to 2746, 2728 to 2747, 2729 to 2748, 2730 to 2749, 2731 to 2750, 2732 to 2751, 2733 to 2752, 2738 to 2757, 2775 to 2794, 2790 to 2809, 2826 to 2845, 2827 to 2846, 2828 to 2847, 2829 to 2848, 2830 to 2849, 2831 to 2850, 2832 to 2851, 2833 to 2852, 2834 to 2853, 2835 to 2854, 2836 to 2855, 2837 to 2856, 2838 to 2857, 2839 to 2858, 2840 to 2859, 2841 to 2860, 2842 to 2861, 2843 to 2862, 2844 to 2863, 2847 to 2866, 2959 to 2978, 2962 to 2981, 2963 to 2982, 3032 to 3051, 3052 to 3071, 3136 to 3155, 3168 to 3187, 3188 to 3207, 3232 to 3251, 3251 to 3270, 3298 to 3317, 3302 to 3321, 3303 to 3322, 3304 to 3323, 3305 to 3324, 3306 to 3325, 3307 to 3326, 3308 to 3327, 3309 to 3328, 3310 to 3329, 3311 to 3330, 3312 to 3331, 3313 to 3332, 3314 to 3333, 3315 to 3334, 3316 to 3335, 3317 to 3336, 3318 to 3337, 3319 to 3338, 3320 to 3339, 3321 to 3340, 3322 to 3341, 3323 to 3342, 3324 to 3343, 3325 to 3344, 3408 to 3427, 3563 to 3582, 3590 to 3609, 3608 to 3627, 3617 to 3636, 3618 to 3637, 3619 to 3638, 3620 to 3639, 3621 to 3640, 3622 to 3641, 3623 to 3642, 3624 to 3643, 3625 to 3644, 3626 to 3645, 3627 to 3646, 3628 to 3647, 3629 to 3648, 3630 to 3649, 3631 to 3650, 3632 to 3651, 3633 to 3652, 3634 to 3653, 3635 to 3654, 3636 to 3655, 3637 to 3656, 3638 to 3657, 3639 to 3658, 3640 to 3659, 3641 to 3660, 3642 to 3661, 3643 to 3662, 3644 to 3663, 3645 to 3664, 3646 to 3665, 3647 to 3666, 3648 to 3667, 3649 to 3668, 3650 to 3669, 3651 to 3670, 3652 to 3671, 3653 to 3672, 3654 to 3673, 3671 to 3690, 3672 to 3691, 3673 to 3692, 3674 to 3693, 3675 to 3694, 3676 to 3695, 3677 to 3696, 3678 to 3697, 3679 to 3698, 3680 to 3699, 3681 to 3700, 3682 to 3701, 3683 to 3702, 3684 to 3703, 3685 to 3704, 3686 to 3705, 3687 to 3706, 3688 to 3707, 3689 to 3708, 3690 to 3709, 3691 to 3710, 3692 to 3711, 3693 to 3712, 3694 to 3713, 3695 to 3714, 3696 to 3715, 3697 to 3716, 3698 to 3717, 3699 to 3718, 3700 to 3719, 3701 to 3720, 3702 to 3721, 3703 to 3722, 3704 to 3723, 3705 to 3724, 3706 to 3725, 3707 to 3726, 3708 to 3727, 3709 to 3728, 3739 to 3758, 3740 to 3759, 3741 to 3760, 3742 to 3761, 3743 to 3762, 3744 to 3763, 3745 to 3764, 3750 to 3769, 3877 to 3896, 3949 to 3968, 3964 to 3983, 3965 to 3984, 3966 to 3985, 3967 to 3986, 3968 to 3987, 3969 to 3988, 3970 to 3989, 3971 to 3990, 3972 to 3991, 3973 to 3992, 3974 to 3993, 3975 to 3994, 3976 to 3995, 3977 to 3996, 3978 to 3997, 3979 to 3998, 3980 to 3999, 3981 to 4000, 3982 to 4001, 3983 to 4002, 3984 to 4003, 3985 to 4004, 3986 to 4005, 3987 to 4006, 3988 to 4007, 3989 to 4008, 3990 to 4009, 3991 to 4010, 3992 to 4011, 3993 to 4012, 3994 to 4013, 3995 to 4014, 3996 to 4015, 3997 to 4016, 3998 to 4017, 3999 to 4018, 4011 to 4030, 4170 to 4189, 4173 to 4192, 4176 to 4195, 4177 to 4196, 4178 to 4197, 4179 to 4198, 4180 to 4199, 4181 to 4200, 4182 to 4201, 4183 to 4202, 4184 to 4203, 4185 to 4204, 4186 to 4205, 4187 to 4206, 4188 to 4207, 4189 to 4208, 4190 to 4209, 4191 to 4210, 4192 to 4211, 4193 to 4212, 4194 to 4213, 4195 to 4214, 4196 to 4215, 4197 to 4216, 4198 to 4217, 4199 to 4218, 4200 to 4219, 4201 to 4220, 4202 to 4221, 4203 to 4222, 4204 to 4223, 4205 to 4224, and 4206 to 4225 of the base sequence of SEQ ID NO: 471.

(5) The antisense oligonucleotide or a pharmaceutically acceptable salt thereof, or a hydrate thereof according to (2-1) or (3), wherein the antisense oligonucleotide is complementary to a nucleic acid comprising at least 15 consecutive bases in a target region selected from the group consisting of positions 14 to 33, 34 to 53, 398 to 427, 723 to 742, 747 to 766, 1148 to 1167, 1289 to 1308, 1484 to 1564, 1578 to 1624, 1686 to 1705, 1748 to 1767, 1768 to 1787, 1794 to 1813, 1877 to 1906, 2093 to 2113, 2151 to 2170, 2206 to 2225, 2239 to 2272, 2287 to 2336, 2337 to 2373, 2387 to 2406, 2613 to 2752, 2775 to 2809, 2826 to 2863, 2962 to 2982, 3032 to 3051, 3052 to 3071, 3168 to 3187, 3232 to 3270, 3302 to 3343, 3408 to 3427, 3590 to 3728, 3739 to 3764, 3877 to 3896, 3949 to 4030, and 4177 to 4225 of the base sequence of SEQ ID NO: 471.

(6) The antisense oligonucleotide or a pharmaceutically acceptable salt thereof, or a hydrate thereof according to (5), wherein the antisense oligonucleotide is complementary to a nucleic acid comprising at least 15 consecutive bases in a target region selected from the group consisting of positions 14 to 33, 34 to 53, 398 to 417, 399 to 418, 400 to 419, 401 to 420, 402 to 421, 403 to 422, 404 to 423, 405 to 424, 406 to 425, 407 to 426, 408 to 427, 723 to 742, 747 to 766, 1148 to 1167, 1289 to 1308, 1484 to 1503, 1488 to 1507, 1491 to 1510, 1493 to 1512, 1506 to 1525, 1517 to 1536, 1531 to 1550, 1545 to 1564, 1578 to 1597, 1579 to 1598, 1580 to 1599, 1581 to 1600, 1582 to 1601, 1583 to 1602, 1584 to 1603, 1585 to 1604, 1586 to 1605, 1587 to 1606, 1588 to 1607, 1589 to 1608, 1590 to 1609, 1591 to 1610, 1592 to 1611, 1593 to 1612, 1594 to 1613, 1595 to 1614, 1596 to 1615, 1597 to 1616, 1598 to 1617, 1599 to 1618, 1600 to 1619, 1601 to 1620, 1602 to 1621, 1603 to 1622, 1604 to 1623, 1605 to 1624, 1686 to 1705, 1748 to 1767, 1768 to 1787, 1794 to 1813, 1877 to 1896, 1878 to 1897, 1879 to 1898, 1880 to 1899, 1881 to 1900, 1882 to 1901, 1883 to 1902, 1884 to 1903, 1885 to 1904, 1886 to 1905, 1887 to 1906, 2093 to 2112, 2094 to 2113, 2151 to 2170, 2206 to 2225, 2239 to 2258, 2253 to 2272, 2287 to 2306, 2301 to 2320, 2317 to 2336, 2337 to 2356, 2354 to 2373, 2387 to 2406, 2613 to 2632, 2614 to 2633, 2615 to 2634, 2616 to 2635, 2617 to 2636, 2618 to 2637, 2619 to 2638, 2620 to 2639, 2621 to 2640, 2622 to 2641, 2623 to 2642, 2624 to 2643, 2625 to 2644, 2626 to 2645, 2627 to 2646, 2628 to 2647, 2629 to 2648, 2630 to 2649, 2631 to 2650, 2632 to 2651, 2633 to 2652, 2634 to 2653, 2635 to 2654, 2636 to 2655, 2637 to 2656, 2638 to 2657, 2639 to 2658, 2640 to 2659, 2641 to 2660, 2642 to 2661, 2643 to 2662, 2644 to 2663, 2645 to 2664, 2646 to 2665, 2647 to 2666, 2648 to 2667, 2649 to 2668, 2663 to 2682, 2672 to 2691, 2673 to 2692, 2674 to 2693, 2675 to 2694, 2676 to 2695, 2677 to 2696, 2678 to 2697, 2679 to 2698, 2680 to 2699, 2681 to 2700, 2682 to 2701, 2683 to 2702, 2684 to 2703, 2685 to 2704, 2686 to 2705, 2687 to 2706, 2688 to 2707, 2689 to 2708, 2690 to 2709, 2691 to 2710, 2692 to 2711, 2693 to 2712, 2694 to 2713, 2695 to 2714, 2696 to 2715, 2697 to 2716, 2698 to 2717, 2699 to 2718, 2700 to 2719, 2701 to 2720, 2702 to 2721, 2703 to 2722, 2704 to 2723, 2705 to 2724, 2706 to 2725, 2707 to 2726, 2708 to 2727, 2709 to 2728, 2710 to 2729, 2711 to 2730, 2712 to 2731, 2713 to 2732, 2714 to 2733, 2715 to 2734, 2716 to 2735, 2717 to 2736, 2718 to 2737, 2719 to 2738, 2720 to 2739, 2721 to 2740, 2722 to 2741, 2723 to 2742, 2724 to 2743, 2725 to 2744, 2726 to 2745, 2727 to 2746, 2728 to 2747, 2729 to 2748, 2730 to 2749, 2731 to 2750, 2732 to 2751, 2733 to 2752, 2775 to 2794, 2790 to 2809, 2826 to 2845, 2827 to 2846, 2828 to 2847, 2829 to 2848, 2830 to 2849, 2831 to 2850, 2832 to 2851, 2833 to 2852, 2834 to 2853, 2835 to 2854, 2836 to 2855, 2837 to 2856, 2838 to 2857, 2839 to 2858, 2840 to 2859, 2841 to 2860, 2842 to 2861, 2843 to 2862, 2844 to 2863, 2962 to 2981, 2963 to 2982, 3032 to 3051, 3052 to 3071, 3168 to 3187, 3232 to 3251, 3251 to 3270, 3302 to 3321, 3303 to 3322, 3304 to 3323, 3305 to 3324, 3306 to 3325, 3307 to 3326, 3308 to 3327, 3309 to 3328, 3310 to 3329, 3311 to 3330, 3312 to 3331, 3313 to 3332, 3314 to 3333, 3315 to 3334, 3316 to 3335, 3317 to 3336, 3318 to 3337, 3319 to 3338, 3320 to 3339, 3321 to 3340, 3322 to 3341, 3323 to 3342, 3324 to 3343, 3408 to 3427, 3590 to 3609, 3608 to 3627, 3617 to 3636, 3618 to 3637, 3619 to 3638, 3620 to 3639, 3621 to 3640, 3622 to 3641, 3623 to 3642, 3624 to 3643, 3625 to 3644, 3626 to 3645, 3627 to 3646, 3628 to 3647, 3629 to 3648, 3630 to 3649, 3631 to 3650, 3632 to 3651, 3633 to 3652, 3634 to 3653, 3635 to 3654, 3636 to 3655, 3637 to 3656, 3638 to 3657, 3639 to 3658, 3640 to 3659, 3641 to 3660, 3642 to 3661, 3643 to 3662, 3644 to 3663, 3645 to 3664, 3646 to 3665, 3647 to 3666, 3648 to 3667, 3649 to 3668, 3650 to 3669, 3651 to 3670, 3652 to 3671, 3653 to 3672, 3654 to 3673, 3671 to 3690, 3672 to 3691, 3673 to 3692, 3674 to 3693, 3675 to 3694, 3676 to 3695, 3677 to 3696, 3678 to 3697, 3679 to 3698, 3680 to 3699, 3681 to 3700, 3682 to 3701, 3683 to 3702, 3684 to 3703, 3685 to 3704, 3686 to 3705, 3687 to 3706, 3688 to 3707, 3689 to 3708, 3690 to 3709, 3691 to 3710, 3692 to 3711, 3693 to 3712, 3694 to 3713, 3695 to 3714, 3696 to 3715, 3697 to 3716, 3698 to 3717, 3699 to 3718, 3700 to 3719, 3701 to 3720, 3702 to 3721, 3703 to 3722, 3704 to 3723, 3705 to 3724, 3706 to 3725, 3707 to 3726, 3708 to 3727, 3709 to 3728, 3739 to 3758, 3740 to 3759, 3741 to 3760, 3742 to 3761, 3743 to 3762, 3744 to 3763, 3745 to 3764, 3877 to 3896, 3949 to 3968, 3964 to 3983, 3965 to 3984, 3966 to 3985, 3967 to 3986, 3968 to 3987, 3969 to 3988, 3970 to 3989, 3971 to 3990, 3972 to 3991, 3973 to 3992, 3974 to 3993, 3975 to 3994, 3976 to 3995, 3977 to 3996, 3978 to 3997, 3979 to 3998, 3980 to 3999, 3981 to 4000, 3982 to 4001, 3983 to 4002, 3984 to 4003, 3985 to 4004, 3986 to 4005, 3987 to 4006, 3988 to 4007, 3989 to 4008, 3990 to 4009, 3991 to 4010, 3992 to 4011, 3993 to 4012, 3994 to 4013, 3995 to 4014, 3996 to 4015, 3997 to 4016, 3998 to 4017, 3999 to 4018, 4011 to 4030, 4177 to 4196, 4178 to 4197, 4179 to 4198, 4180 to 4199, 4181 to 4200, 4182 to 4201, 4183 to 4202, 4184 to 4203, 4185 to 4204, 4186 to 4205, 4187 to 4206, 4188 to 4207, 4189 to 4208, 4190 to 4209, 4191 to 4210, 4192 to 4211, 4193 to 4212, 4194 to 4213, 4195 to 4214, 4196 to 4215, 4197 to 4216, 4198 to 4217, 4199 to 4218, 4200 to 4219, 4201 to 4220, 4202 to 4221, 4203 to 4222, 4204 to 4223, 4205 to 4224, and 4206 to 4225 of the base sequence of SEQ ID NO: 471.

(7) The antisense oligonucleotide or a pharmaceutically acceptable salt thereof, or a hydrate thereof according to (3), wherein the antisense oligonucleotide is complementary to a nucleic acid comprising at least 15 consecutive bases in a target region selected from the group consisting of positions 14 to 33, 34 to 53, 54 to 73, 148 to 167, 397 to 432, 747 to 778, 965 to 984, 1127 to 1146, 1148 to 1173, 1182 to 1213, 1228 to 1254, 1270 to 1308, 1484 to 1564, 1578 to 1629, 1686 to 1705, 1748 to 1767, 1768 to 1787, 1794 to 1813, 1870 to 1913, 2015 to 2034, 2086 to 2118, 2129 to 2148, 2151 to 2170, 2206 to 2225, 2239 to 2272, 2287 to 2336, 2337 to 2373, 2387 to 2406, 2598 to 2632, 2633 to 2668, 2672 to 2691, 2699 to 2757, 2775 to 2809, 2826 to 2866, 2959 to 2978, 3032 to 3051, 3052 to 3071, 3136 to 3155, 3168 to 3187, 3188 to 3207, 3232 to 3270, 3298 to 3317, 3325 to 3344, 3408 to 3427, 3590 to 3690, 3691 to 3728, 3739 to 3769, 3877 to 3896, 3949 to 3983, 3984 to 4030, and 4170 to 4198 of the base sequence of SEQ ID NO: 471.

(8) The antisense oligonucleotide or a pharmaceutically acceptable salt thereof, or a hydrate thereof according to (7), wherein the antisense oligonucleotide is complementary to a nucleic acid comprising at least 15 consecutive bases in a target region selected from the group consisting of positions 14 to 33, 34 to 53, 54 to 73, 148 to 167, 397 to 416, 413 to 432, 747 to 766, 756 to 775, 759 to 778, 965 to 984, 1127 to 1146, 1148 to 1167, 1151 to 1170, 1154 to 1173, 1182 to 1201, 1185 to 1204, 1188 to 1207, 1191 to 1210, 1194 to 1213, 1228 to 1247, 1231 to 1250, 1235 to 1254, 1270 to 1289, 1289 to 1308, 1484 to 1503, 1488 to 1507, 1491 to 1510, 1493 to 1512, 1506 to 1525, 1517 to 1536, 1531 to 1550, 1545 to 1564, 1578 to 1597, 1586 to 1605, 1589 to 1608, 1592 to 1611, 1595 to 1614, 1598 to 1617, 1601 to 1620, 1604 to 1623, 1607 to 1626, 1610 to 1629, 1686 to 1705, 1748 to 1767, 1768 to 1787, 1794 to 1813, 1870 to 1889, 1871 to 1890, 1874 to 1893, 1877 to 1896, 1880 to 1899, 1883 to 1902, 1886 to 1905, 1889 to 1908, 1894 to 1913, 2015 to 2034, 2086 to 2105, 2097 to 2116, 2099 to 2118, 2129 to 2148, 2151 to 2170, 2206 to 2225, 2239 to 2258, 2253 to 2272, 2287 to 2306, 2301 to 2320, 2317 to 2336, 2337 to 2356, 2354 to 2373, 2387 to 2406, 2598 to 2617, 2613 to 2632, 2633 to 2652, 2649 to 2668, 2672 to 2691, 2699 to 2718, 2702 to 2721, 2705 to 2724, 2708 to 2727, 2711 to 2730, 2714 to 2733, 2717 to 2736, 2720 to 2739, 2723 to 2742, 2726 to 2745, 2729 to 2748, 2738 to 2757, 2775 to 2794, 2790 to 2809, 2826 to 2845, 2829 to 2848, 2832 to 2851, 2835 to 2854, 2838 to 2857, 2841 to 2860, 2847 to 2866, 2959 to 2978, 3032 to 3051, 3052 to 3071, 3136 to 3155, 3168 to 3187, 3188 to 3207, 3232 to 3251, 3251 to 3270, 3298 to 3317, 3325 to 3344, 3408 to 3427, 3590 to 3609, 3608 to 3627, 3617 to 3636, 3635 to 3654, 3654 to 3673, 3671 to 3690, 3691 to 3710, 3709 to 3728, 3739 to 3758, 3750 to 3769, 3877 to 3896, 3949 to 3968, 3964 to 3983, 3984 to 4003, 3999 to 4018, 4011 to 4030, 4170 to 4189, 4173 to 4192, 4176 to 4195, and 4179 to 4198 of the base sequence of SEQ ID NO: 471.

(9) The antisense oligonucleotide or a pharmaceutically acceptable salt thereof, or a hydrate thereof according to (5) or (7), wherein the antisense oligonucleotide is complementary to a nucleic acid comprising at least 15 consecutive bases in a target region selected from the group consisting of positions 14 to 33, 34 to 53, 747 to 766, 1148 to 1167, 1289 to 1308, 1484 to 1564, 1578 to 1623, 1686 to 1705, 1748 to 1767, 1768 to 1787, 1794 to 1813, 1877 to 1905, 2151 to 2170, 2206 to 2225, 2239 to 2272, 2287 to 2336, 2337 to 2373, 2387 to 2406, 2613 to 2632, 2633 to 2668, 2672 to 2691, 2699 to 2748, 2775 to 2809, 2826 to 2860, 3032 to 3051, 3052 to 3071, 3168 to 3187, 3232 to 3270, 3408 to 3427, 3590 to 3690, 3691 to 3728, 3739 to 3758, 3877 to 3896, 3949 to 3983, 3984 to 4030, and 4179 to 4198 of the base sequence of SEQ ID NO: 471.

(10-1) The antisense oligonucleotide or a pharmaceutically acceptable salt thereof, or a hydrate thereof according to (9), wherein the antisense oligonucleotide is complementary to a nucleic acid comprising at least 15 consecutive bases in a target region selected from the group consisting of positions 14 to 33, 34 to 53, 747 to 766, 1148 to 1167, 1289 to 1308, 1484 to 1503, 1488 to 1507, 1491 to 1510, 1493 to 1512, 1506 to 1525, 1517 to 1536, 1531 to 1550, 1545 to 1564, 1578 to 1597, 1586 to 1605, 1589 to 1608, 1592 to 1611, 1595 to 1614, 1598 to 1617, 1601 to 1620, 1604 to 1623, 1686 to 1705, 1748 to 1767, 1768 to 1787, 1794 to 1813, 1877 to 1896, 1880 to 1899, 1883 to 1902, 1886 to 1905, 2151 to 2170, 2206 to 2225, 2239 to 2258, 2253 to 2272, 2287 to 2306, 2301 to 2320, 2317 to 2336, 2337 to 2356, 2354 to 2373, 2387 to 2406, 2613 to 2632, 2633 to 2652, 2649 to 2668, 2672 to 2691, 2699 to 2718, 2702 to 2721, 2705 to 2724, 2708 to 2727, 2711 to 2730, 2714 to 2733, 2717 to 2736, 2720 to 2739, 2723 to 2742, 2726 to 2745, 2729 to 2748, 2775 to 2794, 2790 to 2809, 2826 to 2845, 2829 to 2848, 2832 to 2851, 2835 to 2854, 2838 to 2857, 2841 to 2860, 3032 to 3051, 3052 to 3071, 3168 to 3187, 3232 to 3251, 3251 to 3270, 3408 to 3427, 3590 to 3609, 3608 to 3627, 3617 to 3636, 3635 to 3654, 3654 to 3673, 3671 to 3690, 3691 to 3710, 3709 to 3728, 3739 to 3758, 3877 to 3896, 3949 to 3968, 3964 to 3983, 3984 to 4003, 3999 to 4018, 4011 to 4030, and 4179 to 4198 of the base sequence of SEQ ID NO: 471.

(10-2) The antisense oligonucleotide or a pharmaceutically acceptable salt thereof, or a hydrate thereof according to (7), wherein the antisense oligonucleotide is complementary to a nucleic acid comprising at least 15 consecutive bases in a target region selected from the group consisting of positions 397 to 416, 759 to 778, 1127 to 1146, 1148 to 1173, 1182 to 1213, 1488 to 1525, 1586 to 1629, 1686 to 1705, 1768 to 1787, 1870 to 1908, 2097 to 2118, 2151 to 2170, 2239 to 2258, 2287 to 2306, 2317 to 2336, 2633 to 2652, 2702 to 2745, 2832 to 2866, 2959 to 2978, 3298 to 3317, 3635 to 3654, 3671 to 3690, 3691 to 3710, 3750 to 3983, 3984 to 4003, and 4170 to 4198 of the base sequence of SEQ ID NO: 471.

(10-3) The antisense oligonucleotide or a pharmaceutically acceptable salt thereof, or a hydrate thereof according to (7) or (8), wherein the antisense oligonucleotide is complementary to a nucleic acid comprising at least 15 consecutive bases in a target region selected from the group consisting of positions 397 to 416, 759 to 778, 1127 to 1146, 1148 to 1167, 1151 to 1170, 1154 to 1173, 1182 to 1201, 1185 to 1204, 1194 to 1213, 1488 to 1507, 1491 to 1510, 1506 to 1525, 1586 to 1605, 1589 to 1608, 1595 to 1614, 1598 to 1617, 1601 to 1620, 1604 to 1623, 1607 to 1626, 1610 to 1629, 1686 to 1705, 1768 to 1787, 1870 to 1889, 1871 to 1890, 1874 to 1893, 1877 to 1896, 1886 to 1905, 1889 to 1908, 2097 to 2116, 2099 to 2118, 2151 to 2170, 2239 to 2258, 2287 to 2306, 2317 to 2336, 2633 to 2652, 2702 to 2721, 2705 to 2724, 2708 to 2727, 2711 to 2730, 2714 to 2733, 2717 to 2736, 2720 to 2739, 2726 to 2745, 2832 to 2851, 2835 to 2854, 2841 to 2860, 2847 to 2866, 2959 to 2978, 3298 to 3317, 3635 to 3654, 3671 to 3690, 3691 to 3710, 3750 to 3769, 3964 to 3983, 3984 to 4003, 4170 to 4189, 4173 to 4192, 4176 to 4195, and 4179 to 4198 of the base sequence of SEQ ID NO: 471.

(10-4) The antisense oligonucleotide or a pharmaceutically acceptable salt thereof, or a hydrate thereof according to (7), wherein the antisense oligonucleotide is complementary to a nucleic acid comprising at least 15 consecutive bases in a target region selected from the group consisting of positions 397 to 416, 1194 to 1213, 1506 to 1525, 1586 to 1626, 1870 to 1896, 2097 to 2116, 2317 to 2336, 2633 to 2652, 2702 to 2739, 2841 to 2978, 3298 to 3317, 3635 to 3654, 3691 to 3710, 3750 to 3769, 3984 to 4003, and 4170 to 4198 of the base sequence of SEQ ID NO: 471.

(10-5) The antisense oligonucleotide or a pharmaceutically acceptable salt thereof, or a hydrate thereof according to (7) or (8), wherein the antisense oligonucleotide is complementary to a nucleic acid comprising at least 15 consecutive bases in a target region selected from the group consisting of positions 397 to 416, 1194 to 1213, 1506 to 1525, 1586 to 1605, 1595 to 1614, 1598 to 1617, 1604 to 1623, 1607 to 1626, 1870 to 1889, 1871 to 1890, 1874 to 1893, 1877 to 1896, 2097 to 2116, 2317 to 2336, 2633 to 2652, 2702 to 2721, 2708 to 2727, 2711 to 2730, 2714 to 2733, 2717 to 2736, 2720 to 2739, 2841 to 2860, 2959 to 2978, 3298 to 3317, 3635 to 3654, 3691 to 3710, 3750 to 3769, 3984 to 4003, 4170 to 4189, 4173 to 4192, 4176 to 4195, and 4179 to 4198 of the base sequence of SEQ ID NO: 471.

(11) The antisense oligonucleotide or a pharmaceutically acceptable salt thereof, or a hydrate thereof according to (7), wherein the antisense oligonucleotide is complementary to a nucleic acid comprising at least 15 consecutive bases in a target region selected from the group consisting of positions 1598 to 1626, 1870 to 1890, 2097 to 2116, 2711 to 2736, 2841 to 2860, and 4170 to 4198 of the base sequence of SEQ ID NO: 471.

(12) The antisense oligonucleotide or a pharmaceutically acceptable salt thereof, or a hydrate thereof according to (7) or (8), wherein the antisense oligonucleotide is complementary to a nucleic acid comprising at least 15 consecutive bases in a target region selected from the group consisting of positions 1598 to 1617, 1604 to 1623, 1607 to 1626, 1870 to 1889, 1871 to 1890, 2097 to 2116, 2711 to 2730, 2714 to 2733, 2717 to 2736, 2841 to 2860, 4170 to 4189, 4173 to 4192, and 4179 to 4198 of the base sequence of SEQ ID NO: 471.

(12′-1) The antisense oligonucleotide or a pharmaceutically acceptable salt thereof, or a hydrate thereof according to (5), wherein the antisense oligonucleotide is complementary to a nucleic acid comprising at least 15 consecutive bases in a target region selected from the group consisting of positions 400 to 425, 1581 to 1624, 1878 to 1898, 2094 to 2113, 2629 to 2656, 2700 to 2741, 2839 to 2863, 2962 to 2981, 3302 to 3325, 3631 to 3658, 3687 to 3714, 3745 to 3764, 3980 to 4007, 4177 to 4199, and 4204 to 4223 of the base sequence of SEQ ID NO: 471.

(12′-2) The antisense oligonucleotide or a pharmaceutically acceptable salt thereof, or a hydrate thereof according to (5) or (6), wherein the antisense oligonucleotide is complementary to a nucleic acid comprising at least 15 consecutive bases in a target region selected from the group consisting of positions 400 to 419, 403 to 422, 406 to 425, 1581 to 1600, 1582 to 1601, 1583 to 1602, 1584 to 1603, 1585 to 1604, 1587 to 1606, 1588 to 1607, 1590 to 1609, 1591 to 1610, 1592 to 1611, 1593 to 1612, 1594 to 1613, 1596 to 1615, 1597 to 1616, 1599 to 1618, 1600 to 1619, 1602 to 1621, 1603 to 1622, 1605 to 1624, 1878 to 1897, 1879 to 1898, 2094 to 2113, 2629 to 2648, 2637 to 2656, 2700 to 2719, 2701 to 2720, 2703 to 2722, 2704 to 2723, 2706 to 2725, 2707 to 2726, 2709 to 2728, 2710 to 2729, 2712 to 2731, 2713 to 2732, 2715 to 2734, 2716 to 2735, 2718 to 2737, 2719 to 2738, 2721 to 2740, 2722 to 2741, 2839 to 2858, 2840 to 2859, 2842 to 2861, 2843 to 2862, 2844 to 2863, 2962 to 2981, 3302 to 3321, 3306 to 3325, 3631 to 3650, 3632 to 3651, 3633 to 3652, 3634 to 3653, 3636 to 3655, 3637 to 3656, 3638 to 3657, 3639 to 3658, 3687 to 3706, 3695 to 3714, 3745 to 3764, 3980 to 3999, 3988 to 4007, 4177 to 4196, 4178 to 4197, 4180 to 4199, and 4204 to 4223 of the base sequence of SEQ ID NO: 471.

(12′-3) The antisense oligonucleotide or a pharmaceutically acceptable salt thereof, or a hydrate thereof according to (5), wherein the antisense oligonucleotide is complementary to a nucleic acid comprising at least 15 consecutive bases in a target region selected from the group consisting of positions 400 to 425, 1581 to 1624, 1878 to 1898, 2637 to 2656, 2700 to 2741, 2839 to 2863, 2962 to 2981, 3302 to 3325, 3631 to 3658, 3687 to 3706, 3745 to 3764, 4177 to 4199, and 4204 to 4223 of the base sequence of SEQ ID NO: 471.

(12′-4) The antisense oligonucleotide or a pharmaceutically acceptable salt thereof, or a hydrate thereof according to (5) or (6), wherein the antisense oligonucleotide is complementary to a nucleic acid comprising at least 15 consecutive bases in a target region selected from the group consisting of positions 400 to 419, 403 to 422, 406 to 425, 1581 to 1600, 1582 to 1601, 1583 to 1602, 1584 to 1603, 1585 to 1604, 1587 to 1606, 1588 to 1607, 1591 to 1610, 1592 to 1611, 1593 to 1612, 1594 to 1613, 1596 to 1615, 1597 to 1616, 1600 to 1619, 1602 to 1621, 1603 to 1622, 1605 to 1624, 1878 to 1897, 1879 to 1898, 2637 to 2656, 2700 to 2719, 2701 to 2720, 2703 to 2722, 2704 to 2723, 2706 to 2725, 2707 to 2726, 2709 to 2728, 2710 to 2729, 2712 to 2731, 2713 to 2732, 2715 to 2734, 2716 to 2735, 2718 to 2737, 2719 to 2738, 2721 to 2740, 2722 to 2741, 2839 to 2858, 2840 to 2859, 2842 to 2861, 2843 to 2862, 2844 to 2863, 2962 to 2981, 3302 to 3321, 3306 to 3325, 3631 to 3650, 3632 to 3651, 3633 to 3652, 3634 to 3653, 3636 to 3655, 3637 to 3656, 3638 to 3657, 3639 to 3658, 3687 to 3706, 3745 to 3764, 4177 to 4196, 4178 to 4197, 4180 to 4199, and 4204 to 4223 of the base sequence of SEQ ID NO: 471.

(12′-5) The antisense oligonucleotide or a pharmaceutically acceptable salt thereof, or a hydrate thereof according to (5), wherein the antisense oligonucleotide is complementary to a nucleic acid comprising at least 15 consecutive bases in a target region selected from the group consisting of positions 1581 to 1624, 2703 to 2740, 3633 to 3655, and 4177 to 4199 of the base sequence of SEQ ID NO: 471.

(12′-6) The antisense oligonucleotide or a pharmaceutically acceptable salt thereof, or a hydrate thereof according to (5) or (6), wherein the antisense oligonucleotide is complementary to a nucleic acid comprising at least 15 consecutive bases in a target region selected from the group consisting of positions 1581 to 1600, 1582 to 1601, 1584 to 1603, 1585 to 1604, 1587 to 1606, 1588 to 1607, 1594 to 1613, 1596 to 1615, 1597 to 1616, 1602 to 1621, 1603 to 1622, 1605 to 1624, 2703 to 2722, 2707 to 2726, 2709 to 2728, 2710 to 2729, 2712 to 2731, 2713 to 2732, 2715 to 2734, 2716 to 2735, 2718 to 2737, 2719 to 2738, 2721 to 2740, 3633 to 3652, 3634 to 3653, 3636 to 3655, 4177 to 4196, 4178 to 4197, and 4180 to 4199 of the base sequence of SEQ ID NO: 471.

(12′-7) The antisense oligonucleotide or a pharmaceutically acceptable salt thereof, or a hydrate thereof according to (5), wherein the antisense oligonucleotide is complementary to a nucleic acid comprising at least 15 consecutive bases in a target region selected from the group consisting of positions 1587 to 1616, 2712 to 2735, and 4180 to 4199 of the base sequence of SEQ ID NO: 471.

(12′-8) The antisense oligonucleotide or a pharmaceutically acceptable salt thereof, or a hydrate thereof according to (5) or (6), wherein the antisense oligonucleotide is complementary to a nucleic acid comprising at least 15 consecutive bases in a target region selected from the group consisting of positions 1587 to 1606, 1597 to 1616, 2712 to 2731, 2716 to 2735, and 4180 to 4199 of the base sequence of SEQ ID NO: 471.

(13) The antisense oligonucleotide or a pharmaceutically acceptable salt thereof, or a hydrate thereof according to any one of (1) to (12), wherein the antisense oligonucleotide is complementary to a nucleic acid comprising at least 20 consecutive bases in the target region.

(14) The antisense oligonucleotide or a pharmaceutically acceptable salt thereof, or a hydrate thereof according to (3) or (4), comprising:
(i) a base sequence selected from the group consisting of SEQ ID NOs: 1 to 6, 8, 9, 11, 14 to 24, 26, 29, 31 to 60, 66 to 67, 73 to 75, 79 to 82, 86 to 89, 92 to 111, 115 to 136, 138 to 145, 147 to 158, and 160 to 464;
(ii) a base sequence having addition, deletion, or substitution of one or several base(s) in a base sequence selected from the group consisting of SEQ ID NOs: 1 to 6, 8, 9, 11, 14 to 24, 26, 29, 31 to 60, 66 to 67, 73 to 75, 79 to 82, 86 to 89, 92 to 111, 115 to 136, 138 to 145, 147 to 158, and 160 to 464; or
(iii) a base sequence having 90% or more sequence identity with a base sequence selected from the group consisting of SEQ ID NOs: 1 to 6, 8, 9, 11, 14 to 24, 26, 29, 31 to 60, 66 to 67, 73 to 75, 79 to 82, 86 to 89, 92 to 111, 115 to 136, 138 to 145, 147 to 158, and 160 to 464.

(15) The antisense oligonucleotide or a pharmaceutically acceptable salt thereof, or a hydrate thereof according to (5) or (6), comprising:
(i) a base sequence selected from the group consisting of SEQ ID NOs: 1, 5, 6, 14 to 24, 31 to 35, 37 to 40, 42 to 45, 47 to 50, 52 to 59, 73, 92 to 100, 103, 107 to 110, 120 to 136, 138 to 143, 150 to 153, 155 to 158, and 163 to 464;
(ii) a base sequence having addition, deletion, or substitution of one or several base(s) in a base sequence selected from the group consisting of SEQ ID NOs: 1, 5, 6, 14 to 24, 31 to 35, 37 to 40, 42 to 45, 47 to 50, 52 to 59, 73, 92 to 100, 103, 107 to 110, 120 to 136, 138 to 143, 150 to 153, 155 to 158, and 163 to 464; or
(iii) a base sequence having 90% or more sequence identity with a base sequence selected from the group consisting of SEQ ID NOs: 1, 5, 6, 14 to 24, 31 to 35, 37 to 40, 42 to 45, 47 to 50, 52 to 59, 73, 92 to 100, 103, 107 to 110, 120 to 136, 138 to 143, 150 to 153, 155 to 158, and 163 to 464.

(16) The antisense oligonucleotide or a pharmaceutically acceptable salt thereof, or a hydrate thereof according to (7) or (8), comprising:
(i) a base sequence selected from the group consisting of SEQ ID NOs: 1 to 4, 6, 8, 9, 11, 14 to 24, 26, 29, 31 to 37, 39 to 50, 52 to 60, 66 to 67, 73 to 75, 79 to 82, 86 to 89, 92 to 111, 115 to 118, 120 to 136, 138 to 145, 147 to 158, and 160 to 163;
(ii) a base sequence having addition, deletion, or substitution of one or several base(s) in a base sequence selected from the group consisting of SEQ ID NOs: 1 to 4, 6, 8, 9, 11, 14 to 24, 26, 29, 31 to 37, 39 to 50, 52 to 60, 66 to 67, 73 to 75, 79 to 82, 86 to 89, 92 to 111, 115 to 118, 120 to 136, 138 to 145, 147 to 158, and 160 to 163; or
(iii) a base sequence having 90% or more sequence identity with a base sequence selected from the group consisting of SEQ ID NOs: 1 to 4, 6, 8, 9, 11, 14 to 24, 26, 29, 31 to 37, 39 to 50, 52 to 60, 66 to 67, 73 to 75, 79 to 82, 86 to 89, 92 to 111, 115 to 118, 120 to 136, 138 to 145, 147 to 158, and 160 to 163.

(17-1) The antisense oligonucleotide or a pharmaceutically acceptable salt thereof, or a hydrate thereof according to (9) or (10-1), comprising:
(i) a base sequence selected from the group consisting of SEQ ID NOs: 1, 6, 14 to 24, 31 to 35, 37, 39, 40, 42 to 45, 47 to 50, 52 to 59, 73, 92 to 100, 103, 107 to 110, 120 to 136, 138 to 143, 150 to 153, 155 to 158, and 163;
(ii) a base sequence having addition, deletion, or substitution of one or several base(s) in a base sequence selected from the group consisting of SEQ ID NOs: 1, 6, 14 to 24, 31 to 35, 37, 39, 40, 42 to 45, 47 to 50, 52 to 59, 73, 92 to 100, 103, 107 to 110, 120 to 136, 138 to 143, 150 to 153, 155 to 158, and 163; or
(iii) a base sequence having 90% or more sequence identity with a base sequence selected from the group consisting of SEQ ID NOs: 1, 6, 14 to 24, 31 to 35, 37, 39, 40, 42 to 45, 47 to 50, 52 to 59, 73, 92 to 100, 103, 107 to 110, 120 to 136, 138 to 143, 150 to 153, 155 to 158, and 163.

(17-2) The antisense oligonucleotide or a pharmaceutically acceptable salt thereof, or a hydrate thereof according to (10-2) or (10-3), comprising:
(i) a base sequence selected from the group consisting of SEQ ID NOs: 9, 11, 16, 17, 22, 31, 60, 67, 73, 74, 75, 79, 80, 92, 95 to 107, 110, 111, 116, 117, 120 to 122, 126, 128 to 134, 136, 140, 141, 143 to 145, 148, 151 to 154, 156, 157, and 160 to 163;
(ii) a base sequence having addition, deletion, or substitution of one or several base(s) in a base sequence selected from the group consisting of SEQ ID NOs: 9, 11, 16, 17, 22, 31, 60, 67, 73, 74, 75, 79, 80, 92, 95 to 107, 110, 111, 116, 117, 120 to 122, 126, 128 to 134, 136, 140, 141, 143 to 145, 148, 151 to 154, 156, 157, and 160 to 163; or
(iii) a base sequence having 90% or more sequence identity with a base sequence selected from the group consisting of SEQ ID NOs: 9, 11, 16, 17, 22, 31, 60, 67, 73, 74, 75, 79, 80, 92, 95 to 107, 110, 111, 116, 117, 120 to 122, 126, 128 to 134, 136, 140, 141, 143 to 145, 148, 151 to 154, 156, 157, and 160 to 163.

(17-3) The antisense oligonucleotide or a pharmaceutically acceptable salt thereof, or a hydrate thereof according to (10-4) or (10-5), comprising:
(i) a base sequence selected from the group consisting of SEQ ID NOs: 11, 60, 92, 95, 97, 98, 100, 101, 104 to 107, 116, 122, 126, 128, 130 to 134, 143, 145, 148, 151, 153, 154, 157, and 160 to 163;
(ii) a base sequence having addition, deletion, or substitution of one or several base(s) in a base sequence selected from the group consisting of SEQ ID NOs: 11, 60, 92, 95, 97, 98, 100, 101, 104 to 107, 116, 122, 126, 128, 130 to 134, 143, 145, 148, 151, 153, 154, 157, and 160 to 163; or
(iii) a base sequence having 90% or more sequence identity with a base sequence selected from the group consisting of SEQ ID NOs: 11, 60, 92, 95, 97, 98, 100, 101, 104 to 107, 116, 122, 126, 128, 130 to 134, 143, 145, 148, 151, 153, 154, 157, and 160 to 163.

(18) The antisense oligonucleotide or a pharmaceutically acceptable salt thereof, or a hydrate thereof according to (11) or (12), comprising:
(i) a base sequence selected from the group consisting of SEQ ID NOs: 98, 100, 101, 104, 105, 116, 131 to 133, 143, 160, 161, and 163;
(ii) a base sequence having addition, deletion, or substitution of one or several base(s) in a base sequence selected from the group consisting of SEQ ID NOs: 98, 100, 101, 104, 105, 116, 131 to 133, 143, 160, 161, and 163; or
(iii) a base sequence having 90% or more sequence identity with a base sequence selected from the group consisting of SEQ ID NOs: 98, 100, 101, 104, 105, 116, 131 to 133, 143, 160, 161, and 163.

(19-1) The antisense oligonucleotide or a pharmaceutically acceptable salt thereof, or a hydrate thereof according to (12′-1) or (12′-2), comprising:
(i) a base sequence selected from the group consisting of SEQ ID NOs: 21, 164 to 222, 233, 234, and 350 to 355;
(ii) a base sequence having addition, deletion, or substitution of one or several base(s) in a base sequence selected from the group consisting of SEQ ID NOs: 21, 164 to 222, 233, 234, and 350 to 355; or
(iii) a base sequence having 90% or more sequence identity with a base sequence selected from the group consisting of SEQ ID NOs: 21, 164 to 222, 233, 234, and 350 to 355.

(19-2) The antisense oligonucleotide or a pharmaceutically acceptable salt thereof, or a hydrate thereof according to (12′-3) or (12′-4), comprising:
(i) a base sequence selected from the group consisting of SEQ ID NOs: 21, 164 to 171, 173 to 177, 179 to 184, 187 to 214, 216, 219 to 222, 233 to 234, and 350 to 355;
(ii) a base sequence having addition, deletion, or substitution of one or several base(s) in a base sequence selected from the group consisting of SEQ ID NOs: 21, 164 to 171, 173 to 177, 179 to 184, 187 to 214, 216, 219 to 222, 233 to 234, and 350 to 355; or
(iii) a base sequence having 90% or more sequence identity with a base sequence selected from the group consisting of SEQ ID NOs: 21, 164 to 171, 173 to 177, 179 to 184, 187 to 214, 216, 219 to 222, 233 to 234, and 350 to 355.

(19-3) The antisense oligonucleotide or a pharmaceutically acceptable salt thereof, or a hydrate thereof according to (12′-5) or (12′-6), comprising:
(i) a base sequence selected from the group consisting of SEQ ID NOs: 167 to 171, 175 to 177, 180 to 182, 190, 193 to 202, 219 to 221, 233, and 351 to 353;
(ii) a base sequence having addition, deletion, or substitution of one or several base(s) in a base sequence selected from the group consisting of SEQ ID NOs: 167 to 171, 175 to 177, 180 to 182, 190, 193 to 202, 219 to 221, 233, and 351 to 353; or
(iii) a base sequence having 90% or more sequence identity with a base sequence selected from the group consisting of SEQ ID NOs: 167 to 171, 175 to 177, 180 to 182, 190, 193 to 202, 219 to 221, 233, and 351 to 353.

(20) The antisense oligonucleotide or a pharmaceutically acceptable salt thereof, or a hydrate thereof according to (12′-7) or (12′-8), comprising:
(i) a base sequence selected from the group consisting of SEQ ID NOs: 170, 177, 196, 199, and 221;
(ii) a base sequence having addition, deletion, or substitution of one or several base(s) in a base sequence selected from the group consisting of SEQ ID NOs: 170, 177, 196, 199, and 221; or
(iii) a base sequence having 90% or more sequence identity with a base sequence selected from the group consisting of SEQ ID NOs: 170, 177, 196, 199, and 221.

(21) The antisense oligonucleotide or a pharmaceutically acceptable salt thereof, or a hydrate thereof according to any one of (14) to (20), comprising the base sequence (i).

(22) An antisense oligonucleotide consisting of 15 to 22 nucleotides complementary to a nucleic acid comprising at least 15 consecutive bases in a target region selected from the group consisting of positions 468 to 487, 474 to 493, 583 to 602, 586 to 605, 619 to 638, 770 to 789, 774 to 793, 775 to 794, 778 to 797, 804 to 823, 851 to 870, 1160 to 1179, 1162 to 1181, 1170 to 1189, 1173 to 1192, 1205 to 1224, 1210 to 1229, 1216 to 1235, 1217 to 1236, 1219 to 1238, 1385 to 1404, 1441 to 1460, 1818 to 1837, 1902 to 1921, 1905 to 1924, 1908 to 1927, 1914 to 1933, 1931 to 1950, 2117 to 2136, 2749 to 2768, 3100 to 3119, and 4125 to 4144 of a base sequence of SEQ ID NO: 471, or a pharmaceutically acceptable salt thereof, or a hydrate thereof.

(23) The antisense oligonucleotide or a pharmaceutically acceptable salt thereof, or a hydrate thereof according to (22), comprising:
(i) a base sequence selected from the group consisting of SEQ ID NOs: 7, 10, 12, 13, 25, 27, 28, 30, 61 to 65, 68 to 72, 76 to 78, 83 to 85, 90, 91, 112 to 114, 137, 146, and 159;
(ii) a base sequence having addition, deletion, or substitution of one or several base(s) in a base sequence selected from the group consisting of SEQ ID NOs: 7, 10, 12, 13, 25, 27, 28, 30, 61 to 65, 68 to 72, 76 to 78, 83 to 85, 90, 91, 112 to 114, 137, 146, and 159; or
(iii) a base sequence having 90% or more sequence identity with a base sequence selected from the group consisting of SEQ ID NOs: 7, 10, 12, 13, 25, 27, 28, 30, 61 to 65, 68 to 72, 76 to 78, 83 to 85, 90, 91, 112 to 114, 137, 146, and 159.

(24) The antisense oligonucleotide or a pharmaceutically acceptable salt thereof, or a hydrate thereof according to (23), comprising the base sequence (i).

(25) The antisense oligonucleotide or a pharmaceutically acceptable salt thereof, or a hydrate thereof according to any one of (1) to (24), wherein the antisense oligonucleotide consists of 20 nucleotides.

(26) The antisense oligonucleotide or a pharmaceutically acceptable salt thereof, or a hydrate thereof according to any one of (1) to (25), wherein a sugar moiety and/or a phosphate-binding region of at least one nucleotide constituting the oligonucleotide is modified.

(27-1) The antisense oligonucleotide or a pharmaceutically acceptable salt thereof, or a hydrate thereof according to (26), wherein the antisense oligonucleotide is a gapmer comprising a gap region at the center, and two wing regions adjacent to the gap region on the 5′ end side and the 3′ end side (a 5′ wing region and a 3′ wing region).

(27-2) The antisense oligonucleotide or a pharmaceutically acceptable salt thereof, or a hydrate thereof according to (27-1), wherein the antisense oligonucleotide comprises, from the 5′ to 3′ direction, a 5′ wing region of 5 nucleotides long, a gap region of 10 nucleotides long, and a 3′ wing region of 5 nucleotides long.

(28) The antisense oligonucleotide or a pharmaceutically acceptable salt thereof, or a hydrate thereof according to (27-1) or (27-2), wherein bonds between nucleosides are all phosphorothioate bonds.

(29) The antisense oligonucleotide or a pharmaceutically acceptable salt thereof, or a hydrate thereof according to (27-1) or (27-2), wherein one or more of a bond between the 2nd and 3rd nucleosides, a bond between the 3rd and 4th nucleosides, and a bond between the 4th and 5th nucleosides from the 5′ side of the 5′ wing region are phosphodiester bonds, and/or one or more of a bond between the 1st and 2nd nucleosides, a bond between the 2nd and 3rd nucleosides, and a bond between the 3rd and 4th nucleosides from the 5′ side of the 3′ wing region are phosphodiester bonds, and bonds between the other nucleosides are all phosphorothioate bonds.

(30) The antisense oligonucleotide or a pharmaceutically acceptable salt thereof, or a hydrate thereof according to (29), wherein the bond between the 2nd and 3rd nucleosides, and the bond between the 4th and 5th nucleosides from the 5′ side of the 5′ wing region are phosphodiester bonds, and/or the bond between the 1st and 2nd nucleosides, and the bond between the 3rd and 4th nucleosides from the 5′ side of the 3′ wing region are phosphodiester bonds, and bonds between the other nucleosides are all phosphorothioate bonds.

(31) The antisense oligonucleotide or a pharmaceutically acceptable salt thereof, or a hydrate thereof according to (29), wherein the bond between the 2nd and 3rd nucleosides, the bond between the 3rd and 4th nucleosides, and the bond between the 4th and 5th nucleosides from the 5′ side of the 5′ wing region are phosphodiester bonds, and/or the bond between the 1st and 2nd nucleosides, the bond between the 2nd and 3rd nucleosides, and the bond between the 3rd and 4th nucleosides from the 5′ side of the 3′ wing region are phosphodiester bonds, and bonds between the other nucleosides are all phosphorothioate bonds.

(32) The antisense oligonucleotide or a pharmaceutically acceptable salt thereof, or a hydrate thereof according to any one of (27-1) to (31), wherein the wing region comprises a 2′-OMe (2′-O—CH₃) group and/or a 2′-O-MOE (2′-O—CH₂CH₂OCH₃) group.

(33) A pharmaceutical composition comprising the antisense oligonucleotide or a pharmaceutically acceptable salt thereof, or a hydrate thereof according to any one of (1) to (32).

(34) The antisense oligonucleotide or a pharmaceutically acceptable salt thereof, or a hydrate thereof according to any one of (1) to (32), or the pharmaceutical composition according to (33), for treating and/or preventing dentatorubral-pallidoluysian atrophy (DRPLA).

(35) A method for treating and/or preventing dentatorubral-pallidoluysian atrophy (DRPLA) comprising administering, to a subject, the antisense oligonucleotide or a pharmaceutically acceptable salt thereof, or a hydrate thereof according to any one of (1) to (32), or the pharmaceutical composition according to (33) or (34).

The present invention provides an antisense oligonucleotide targeting an ATN1 mRNA or pre-mRNA, or a pharmaceutically acceptable salt thereof, or a hydrate thereof, and a composition or the like comprising the antisense oligonucleotide or a pharmaceutically acceptable salt thereof, or a hydrate thereof.

Since mutant ATN1 that is a causative gene for DRPLA is directly inhibited in the present invention, a DRPLA therapeutic agent having high treatment satisfaction may be provided. In the present invention, since the antisense oligonucleotide can be designed with targeting mutant ATN1 that is a causative gene for DRPLA, a DRPLA therapeutic agent with few side effects may be provided in a preferred embodiment of the present invention. In one embodiment of the present invention, personalized medicine based on genetic information of individual patients can be provided.

### DESCRIPTION OF EMBODIMENTS

In one embodiment, the present invention relates to an antisense oligonucleotide consisting of 15 to 22 nucleotides complementary to a nucleic acid comprising at least 15 consecutive bases in a target region selected from the group consisting of positions 1 to 84, 142 to 168, 197 to 224, 286 to 317, 324 to 370, 391 to 434, 519 to 540, 623 to 643, 690 to 780, 824 to 855, 860 to 897, 948 to 987, 1044 to 1072, 1125 to 1174, 1181 to 1213, 1228 to 1255, 1265 to 1327, 1334 to 1356, 1416 to 1440, 1447 to 1631, 1638 to 1667, 1675 to 1705, 1748 to 1823, 1838 to 1861, 1870 to 1913, 1920 to 1941, 2000 to 2040, 2047 to 2075, 2086 to 2120, 2129 to 2187, 2194 to 2415, 2451 to 2497, 2592 to 2759, 2766 to 2870, 2928 to 2948, 2955 to 2989, 3021 to 3086, 3133 to 3209, 3217 to 3284, 3295 to 3350, 3384 to 3436, 3562 to 3771, 3858 to 3905, 3931 to 4039, 4067 to 4134, 4170 to 4232, 4241 to 4283, and 4286 to 4355 of a base sequence of SEQ ID NO: 471, or a pharmaceutically acceptable salt thereof, or a hydrate thereof.

SEQ ID NO: 471 is a sequence of mRNA of human atrophin-1 (ATN1) (Gen Bank: NM_001007026). The sequence of SEQ ID NO: 471 is a base sequence comprising 4355 nucleotides, and a region consisting of positions 1691 to 1747 of SEQ ID NO: 1 corresponds to a repeat region comprising CAG/CAA repeat sequences. In one embodiment, a target sequence of the antisense oligonucleotide of the present invention does not comprise a repeat region comprising the CAG/CAA repeat sequences.

In one embodiment, the antisense oligonucleotide of the present invention is complementary to a nucleic acid comprising or consisting of at least 15, at least 16, at least 17, at least 18, at least 19, at least 20, and for example 20 consecutive bases in the target region.

As used herein, an antisense oligonucleotide “complementary” to a given nucleic acid is not limited to an antisense oligonucleotide that forms Watson-Crick base pairs with the intended nucleic acid, but also includes an antisense oligonucleotide that forms wobble base pairs therewith. Herein, the Watson-Crick base pair means a base pair that forms a hydrogen bond between adenine and thymine, between adenine and uracil, or between guanine and cytosine, and the wobble base pair means a base pair that forms a hydrogen bond between guanine and uracil, between inosine and uracil, between inosine and adenine, or between inosine and cytosine. The term “complementary base sequence” does not have to have 100% complementarity with the intended base sequence, and may comprise, for example, 1, 2, 3, 4, or 5 noncomplementary nucleotides based on the intended base sequence, or may be a base sequence shorter by 1 nucleotide, 2 nucleotides, 3 nucleotides, 4 nucleotides, or 5 nucleotides than the intended base sequence. In one embodiment, an antisense oligonucleotide “complementary” to a given nucleic acid has at least 90%, 91%, 92%, 93%, 94%, 95%, 96%, 97%, 98%, 99%, or 100% complementarity with the intended nucleic acid. Complementarity can be easily determined by those skilled in the art, and can be calculated, for example, by aligning two sequences, counting the number of nucleotides forming Watson-Crick base pairs or wobble base pairs between these sequences, dividing the number of nucleotides forming the base pairs by the total number of nucleotides of the sequence, and multiplying the resultant by 100.

An example of an antisense oligonucleotide “complementary” to a given nucleic acid includes an antisense oligonucleotide that can hybridize under stringent conditions to the nucleic acid. As used herein, the term “stringent conditions” may be any of low stringent conditions, moderate stringent conditions, and high stringent conditions. The term “low stringent conditions” is conditions of, for example, 5×SSC, 5×Denhardt's solution, 0.5% SDS, 50% formamide at 32° C. The term “moderate stringent conditions” is conditions of, for example, 5×SSC, 5×Denhardt's solution, 0.5% SDS, 50% formamide at 42° C., or 5×SSC, 1% SDS, 50 mM Tris-HCl (pH 7.5), 50% formamide at 42° C. The term “high stringent conditions” is conditions of, for example, 5×SSC, 5×Denhardt's solution, 0.5% SDS, 50% formamide at 50° C., or 0.2×SSC, 0.1% SDS at 65° C. Under these conditions, base sequences with higher sequence identity are expected to be obtained efficiently at higher temperatures. Multiple factors are, however, involved in hybridization stringency including temperature, probe concentration, probe length, ionic strength, time, salt concentration and others, and those skilled in the art may appropriately select these factors to achieve similar stringency.

When commercially available kits are used for hybridization, for example, an AlkPhos Direct Labelling and Detection System (GE Healthcare) may be used. In this case, according to the attached protocol, after incubation with a labeled probe overnight, the membrane can be washed with a primary wash buffer containing 0.1% (w/v) SDS at 55° C., thereby detecting hybridization. Alternatively, when a probe is labeled with digoxigenin (DIG) using a commercially available reagent (e.g., a PCR Labelling Mix (Roche Diagnostics)) in producing the probe based on a target sequence, hybridization can be detected with a DIG Nucleic Acid Detection Kit (Roche Diagnostics) or the like.

The identity between base sequences may be determined using algorithm BLAST (Basic Local Alignment Search Tool) by Karlin and Altschul (Proc. Natl. Acad. Sci. USA 872264-2268, 1990; Proc. Natl. Acad. Sci. USA 90: 5873, 1993). Programs called BLASTN and BLASTX based on the BLAST algorithm have been developed (Altschul S F, et al.: J. Mol. Biol. 215: 403, 1990). When a base sequence is analyzed using BLASTN, the parameters are, for example, score=100 and wordlength=12. When BLAST and Gapped BLAST programs are used, the default parameters for each program are employed.

In one embodiment, the antisense oligonucleotide of the present invention may be, for example, 15 or more nucleotides long, 16 or more nucleotides long, 17 or more nucleotides long, 18 or more nucleotides long, 19 or more nucleotides long, 20 or more nucleotides long, 21 or more nucleotides long, 22 or more nucleotides long, 23 or more nucleotides long, 24 or more nucleotides long, 25 or more nucleotides long, 26 or more nucleotides long, 27 or more nucleotides long, 28 or more nucleotides long, 29 or more nucleotides long, or 30 nucleotides long, and may be 30 or less nucleotides long, 29 or less nucleotides long, 28 or less nucleotides long, 27 or less nucleotides long, 26 or less nucleotides long, 25 or less nucleotides long, 24 or less nucleotides long, 23 or less nucleotides long, 22 or less nucleotides long, 21 or less nucleotides long, 20 or less nucleotides long, 19 or less nucleotides long, 18 or less nucleotides long, 17 or less nucleotides long, 16 or less nucleotides long, or 15 nucleotides long. The antisense oligonucleotide of the present invention may consist of, for example, 15 to 30 nucleotides, 15 to 25 nucleotides, 15 to 24 nucleotides, 15 to 23 nucleotides, 15 to 22 nucleotides, 15 to 21 nucleotides, 15 to 20 nucleotides, 16 to 25 nucleotides, 17 to 25 nucleotides, 18 to 25 nucleotides, 19 to 25 nucleotides, 20 to 25 nucleotides, 16 to 24 nucleotides, 17 to 23 nucleotides, 18 to 22 nucleotides, 19 to 21 nucleotides, and for example, 20 nucleotides.

Examples of the pharmaceutically acceptable salt of the antisense oligonucleotide of the present invention are alkali metal salts such as salts of sodium, potassium and lithium; alkaline earth metal salts such as salts of calcium and magnesium; metal salts such as salts of aluminum, iron, zinc, copper, nickel, cobalt, etc.; ammonium salts; organic amine salts such as salts of t-octylamine, dibenzylamine, morpholine, glucosamine, phenylglycine alkyl ester, ethylenediamine, N-methylglucamine, guanidine, diethylamine, triethylamine, dicyclohexylamine, N,N′-dibenzylethylenediamine, chloroprocaine, procaine, diethanolamine, N-benzylphenethylamine, piperazine, tetramethylammonium, and tris(hydroxymethyl)aminomethane; hydrohalide salts such as salts of hydrofluorates, hydrochlorides, hydrobromides, and hydroiodides; inorganic acid salts such as nitrates, perchlorates, sulfates, and phosphates; lower alkane sulfonates such as methanesulfonates, trifluoromethanesulfonates and ethanesulfonates; arylsulfonates such as benzenesulfonates, and p-toluenesulfonates; organic acid salts such as acetates, malates, fumarates, succinates, citrates, tartrates, oxalates, and maleates; and amino acid salts such as salts of glycine, lysine, arginine, ornithine, glutamic acid, and aspartic acid. These salts may be produced by known methods. Alternatively, the antisense oligonucleotide of the present invention may be in the form of a hydrate thereof.

The antisense oligonucleotide of the present invention is composed of nucleotides as constituent units, and such nucleotides may be any of ribonucleotides, deoxyribonucleotides, and modified nucleotides.

The modified nucleotide refers to one fully or partly modified in a nucleobase, a sugar moiety and a phosphate-binding region that constitute the ribonucleotide or deoxyribonucleotide.

The nucleobase includes, for example, adenine, guanine, hypoxanthine, cytosine, thymine, uracil, and modified bases thereof. Examples of such modified bases include, but are not limited to, pseudouracil, 3-methyluracil, dihydrouracil, 5-alkylcytosines (e.g., 5-methylcytosine), 5-alkyluracils (e.g., 5-ethyluracil), 5-halouracils (e.g., 5-bromouracil), 6-azapyrimidine, 6-alkylpyrimidines (e.g., 6-methyluracil), 2-thiouracil, 4-thiouracil, 4-acetylcytosine, 5-(carboxyhydroxymethyl) uracil, 5-carboxymethylaminomethyl-2-thiouracil, 5-carboxymethylaminomethyluracil, 1-methyladenine, 1-methylhypoxanthine, 2,2-dimethylguanine, 3-methylcytosine, 2-methyladenine, 2-methylguanine, N6-methyladenine, 7-methylguanine, 5-methoxyaminomethyl-2-thiouracil, 5-methylaminomethyluracil, 5-methylcarbonylmethyluracil, 5-methyloxyuracil, 5-methyl-2-thiouracil, 2-methylthio-N6-isopentenyladenine, uracil-5-oxyacetic acid, 2-thiocytosine, purine, 2,6-diaminopurine, 2-aminopurine, isoguanine, indole, imidazole, and xanthine.

As used herein, thymine “T” and uracil “U” are interchangeable with each other. Neither “T” nor “U” essentially influences the activity of the antisense oligonucleotide of the present invention, and therefore, in the base sequences shown in this specification, cases where “T” is “U” are also included, and these are indicated by the same sequence number. Besides, as used herein, a sequence comprising a modified base and a sequence not comprising the modified base are represented by the same SEQ ID NO. For example, “cytosine” and “methylcytosine” are interchangeable with each other, and cases where “cytosine” is “methylcytosine” are also included, and these are indicated by the same sequence number.

Modification of the sugar moiety may include, for example, modifications at the 2′-position of ribose, and modifications of the other portions of the sugar. The modification at the 2′-position of ribose includes a modification of replacing the 2′-OH of ribose with —OR, —OROR, —R, —R′OR, —SH, —SR, —NH₂, —NHR, —NR₂, —N₃, —CN, —F, —Cl, —Br or —I, for example, —OMe(-O—CH₃) or —O-methoxyethyl (—O-MOE: —O—CH₂CH₂OCH₃). Here, R represents an alkyl, a cycloalkyl, an acyl, or an aryl, and R′ represents an alkylene.

The modification for the other portions of the sugar includes, for example, replacement of O at the 4′-position of ribose or deoxyribose with S, bridging between 2′- and 4′-positions of the sugar, such as LNA (locked nucleic acid) or ENA (2′-O,4′-C-ethylene-bridged nucleic acids), but is not limited thereto.

The modification for the phosphate-binding region includes, for example, a modification of replacing phosphodiester bond with a phosphorothioate bond, a phosphorodithioate bond, an alkyl phosphonate bond, a phosphoramidate bond, or a boranophosphate bond (cf., e.g., Enya et al.: Bioorganic & Medicinal Chemistry, 2008, 18, 9154-9160) (cf., e.g., Japan Domestic Re-Publications of PCT Application Nos. 2006/129594 and 2006/038608).

As used herein, the alkyl is preferably a straight or branched alkyl having 1 to 6 carbon atoms. Specific examples include methyl, ethyl, n-propyl, isopropyl, n-butyl, isobutyl, sec-butyl, tert-butyl, n-pentyl, isopentyl, neopentyl, tert-pentyl, n-hexyl and isohexyl. The alkyl may optionally be substituted, and examples of such substituents are a halogen, an alkoxy, a cyano and a nitro. The alkyl may be substituted with 1 to 3 substituents.

As used herein, the cycloalkyl is preferably a cycloalkyl having 3 to 12 carbon atoms. Specific examples include cyclopropyl, cyclobutyl, cyclopentyl, cyclohexyl, cycloheptyl, cyclooctyl, cyclodecyl, and cyclododecyl.

As used herein, examples of the halogen include fluorine, chlorine, bromine, and iodine.

As used herein, the alkoxy is a straight or branched alkoxy having 1 to 6 carbon atoms such as methoxy, ethoxy, n-propoxy, isopropoxy, n-butoxy, isobutoxy, sec-butoxy, tert-butoxy, n-pentyloxy, isopentyloxy, n-hexyloxy, and isohexyloxy. Among others, an alkoxy having 1 to 3 carbon atoms is preferred.

As used herein, the aryl is preferably an aryl having 6 to 10 carbon atoms. Specific examples include phenyl, α-naphthyl, and β-naphthyl. Among others, phenyl is preferred. The aryl may optionally be substituted, and examples of such substituents include an alkyl, a halogen, an alkoxy, a cyano and a nitro. The aryl may be substituted with 1 to 3 substituents.

As used herein, the alkylene is preferably a straight or branched alkylene having 1 to 6 carbon atoms. Specific examples include methylene, ethylene, trimethylene, tetramethylene, pentamethylene, hexamethylene, 2-(ethyl) trimethylene, and 1-(methyl) tetramethylene.

As used herein, the acyl includes a straight or branched alkanoyl or aroyl. Examples of the alkanoyl include formyl, acetyl, 2-methylacetyl, 2,2-dimethylacetyl, propionyl, butyryl, isobutyryl, pentanoyl, 2,2-dimethylpropionyl, and hexanoyl. Examples of the aroyl include benzoyl, toluoyl, and naphthoyl. The aroyl may optionally be substituted at substitutable positions, and may be substituted with an alkyl(s).

In one embodiment, the antisense oligonucleotide of the present invention is a gapmer comprising a gap region at the center, and two wing regions adjacent to the gap region on the 5′ end side and the 3′ end side (referred to also as the 5′ wing region and the 3′ wing region, respectively). The gap region is a region recognized by RNase H, and having a sugar moiety constituted by deoxyribonucleotide not modified. The wing region contains at least one modified nucleotide, and is, for example, fully constituted by a modified nucleotide (e.g., a ribonucleotide having modification at the 2′-position of ribose). In one embodiment, each of nucleosides of the 5′ wing region and the 3′ wing region comprises at least one, for example, two or more, three or more, four or more, or five or more modified sugar moieties, such as a 2′-OMe group and/or a 2′-O-MOE group, and for example, all nucleosides of the 5′ wing region and the 3′ wing region may comprise a 2′-OMe group and/or a 2′-O-MOE group. Besides, the nucleosides of the 5′ wing region and the 3′ wing region may comprise a modification in the base moiety, and may comprise, for example, at least one methylcytosine.

The length of the gap region is not limited, and may be, for example, 5 to 15, 8 to 12, 9 to 11, or 10 nucleotides long. The lengths of the 5′ wing region and the 3′ wing region are not limited, and may be, for example, independently 2 to 10, 3 to 8, 4 to 6, or 5 nucleotides long. In one embodiment, the length of the gap region is 10 nucleotides long, and the lengths of the 5′ wing region and the 3′ wing region are 5 nucleotides long.

In one embodiment, the gapmer of the present invention comprises one or more modifications of phosphate-binding regions, e.g., phosphorothioate bonds, and for example, one or more, two or more, three or more, four or more, five or more, ten or more, fifteen or more, and for example, all of bonds between nucleotides may be phosphorothioate bonds. In one embodiment, bonds between nucleosides are all phosphorothioate bonds in the gapmer of the present invention. In one embodiment, the gapmer of the present invention comprises, for example, in the 5′ wing region and/or the 3′ wing region, one or more, two or more, three or more, four or more, five or more, six or more, seven or more, eight or more, nine or more, or ten or more (e.g., two to six) phosphodiester bonds, and bonds between the other nucleosides are all phosphorothioate bonds. In one embodiment, one or more of a bond between the 2nd and 3rd nucleosides, a bond between the 3rd and 4th nucleosides, and a bond between the 4th and 5th nucleosides from the 5′ side of the 5′ wing region are phosphodiester bonds, and/or one or more of a bond between the 1st and 2nd nucleosides, a bond between the 2nd and 3rd nucleosides, and a bond between the 3rd and 4th nucleosides from the 5′ side of the 3′ wing region are phosphodiester bonds, and bonds between the other nucleosides are all phosphorothioate bonds. In one embodiment, the bond between the 2nd and 3rd nucleosides, and the bond between the 4th and 5th nucleosides from the 5′ side of the 5′ wing region are phosphodiester bonds, and/or the bond between the 1st and 2nd nucleosides, and the bond between the 3rd and 4th nucleosides from the 5′ side of the 3′ wing region are phosphodiester bonds, and bonds between the other nucleosides are all phosphorothioate bonds. In one embodiment, the bond between the 2nd and 3rd nucleosides, the bond between the 3rd and 4th nucleosides, and the bond between the 4th and 5th nucleosides from the 5′ side of the 5′ wing region are phosphodiester bonds, and/or the bond between the 1st and 2nd nucleosides, the bond between the 2nd and 3rd nucleosides, and the bond between the 3rd and 4th nucleosides from the 5′ side of the 3′ wing region are phosphodiester bonds, and bonds between the other nucleosides are all phosphorothioate bonds. In one embodiment, the bond between the 2nd and 3rd nucleosides, and the bond between the 3rd and 4th nucleosides from the 5′ side of the 5′ wing region are phosphodiester bonds, and/or the bond between the 2nd and 3rd nucleosides, and the bond between the 3rd and 4th nucleosides from the 5′ side of the 3′ wing region are phosphodiester bonds, and bonds between the other nucleosides are all phosphorothioate bonds. In one embodiment, the bond between the 3rd and 4th nucleosides, and the bond between the 4th and 5th nucleosides from the 5′ side of the 5′ wing region are phosphodiester bonds, and/or the bond between the 1st and 2nd nucleosides, and the bond between the 2nd and the 3rd nucleosides from the 5′ side of the 3′ wing region are phosphodiester bonds, and bonds between the other nucleosides are all phosphorothioate bonds. In one embodiment, the bond between the 2nd and 3rd nucleosides from the 5′ side of the 5′ wing region is a phosphodiester bond, and/or the bond between the 3rd and 4th nucleosides from the 5′ side of the 3′ wing region is a phosphodiester bond, and bonds between the other nucleosides are all phosphorothioate bonds. In one embodiment, the bond between the 3rd and 4th nucleosides from the 5′ side of the 5′ wing region is a phosphodiester bond, and/or the bond between the 2nd and 3rd nucleosides from the 5′ side of the 3′ wing region is a phosphodiester bond, and bonds between the other nucleosides are all phosphorothioate bonds. In one embodiment, the bond between the 4th and 5th nucleosides from the 5′ side of the 5′ wing region is a phosphodiester bonds, and/or the bond between the 1st and 2nd nucleosides from the 5′ side of the 3′ wing region is a phosphodiester bond, and bonds between the other nucleosides are all phosphorothioate bonds. In one embodiment, the gapmer of the present invention does not comprise a modification of a phosphate-binding region, and bonds between nucleosides may be all phosphodiester bonds.

In one embodiment, the antisense oligonucleotide of the present invention is complementary to a nucleic acid comprising at least 15 consecutive bases in a target region selected from the group consisting of positions 1 to 84, 142 to 168, 197 to 224, 286 to 317, 324 to 370, 391 to 434, 519 to 540, 623 to 643, 690 to 780, 824 to 855, 860 to 897, 948 to 987, 1044 to 1072, 1125 to 1174, 1181 to 1213, 1228 to 1255, 1265 to 1327, 1334 to 1356, 1416 to 1440, 1447 to 1631, 1638 to 1667, 1675 to 1705, 1748 to 1823, 1838 to 1861, 1870 to 1913, 1920 to 1941, 2000 to 2040, 2047 to 2075, 2086 to 2120, 2129 to 2187, 2194 to 2415, 2451 to 2497, 2592 to 2759, 2766 to 2870, 2928 to 2948, 2955 to 2989, 3021 to 3086, 3133 to 3209, 3217 to 3284, 3295 to 3350, 3384 to 3436, 3562 to 3771, 3858 to 3905, 3931 to 4039, 4067 to 4134, 4170 to 4232, 4241 to 4283, and 4286 to 4355 of the base sequence of SEQ ID NO: 471.

In one embodiment, the antisense oligonucleotide of the present invention is complementary to a nucleic acid comprising at least 15 consecutive bases in a target region selected from the group consisting of positions 1 to 84, 142 to 168, 391 to 434, 697 to 780, 962 to 980, 1125 to 1174, 1181 to 1213, 1228 to 1255, 1265 to 1320, 1454 to 1631, 1682 to 1705, 1748 to 1816, 1870 to 1913, 2015 to 2040, 2086 to 2120, 2129 to 2187, 2201 to 2408, 2592 to 2759, 2773 to 2870, 2955 to 2982, 3028 to 3079, 3133 to 3209, 3224 to 3277, 3295 to 3350, 3391 to 3429, 3562 to 3771, 3865 to 3898, 3938 to 4032, 4074 to 4127, and 4170 to 4225 of the base sequence of SEQ ID NO: 471.

In one embodiment, the antisense oligonucleotide of the present invention is complementary to a nucleic acid comprising at least 15 consecutive bases in a target region selected from the group consisting of positions 1 to 69, 331 to 363, 398 to 427, 697 to 773, 867 to 890, 955 to 980, 1132 to 1167, 1272 to 1320, 1454 to 1624, 1682 to 1705, 1748 to 1816, 1877 to 1906, 2007 to 2033, 2093 to 2113, 2136 to 2180, 2201 to 2408, 2458 to 2490, 2599 to 2752, 2773 to 2863, 2962 to 2982, 3028 to 3079, 3140 to 3202, 3224 to 3277, 3302 to 3343, 3391 to 3429, 3569 to 3764, 3865 to 3898, 3938 to 4032, 4074 to 4127, 4177 to 4225, 4248 to 4276, and 4293 to 4355 of the base sequence of SEQ ID NO: 471.

In one embodiment, the antisense oligonucleotide of the present invention is complementary to a nucleic acid comprising at least 15 consecutive bases in a target region selected from the group consisting of positions 1 to 69, 398 to 427, 697 to 773, 1132 to 1167, 1272 to 1320, 1454 to 1624, 1682 to 1705, 1748 to 1816, 1877 to 1906, 2093 to 2113, 2136 to 2180, 2201 to 2408, 2599 to 2752, 2773 to 2863, 2962 to 2982, 3028 to 3079, 3140 to 3202, 3224 to 3277, 3302 to 3343, 3391 to 3429, 3569 to 3764, 3865 to 3898, 3938 to 4032, and 4177 to 4225 of the base sequence of SEQ ID NO: 471.

In one embodiment, the antisense oligonucleotide of the present invention is complementary to a nucleic acid comprising at least 15 consecutive bases in a target region selected from the group consisting of positions 14 to 33, 34 to 53, 54 to 73, 148 to 167, 397 to 432, 723 to 742, 747 to 778, 965 to 984, 1127 to 1146, 1148 to 1173, 1182 to 1213, 1228 to 1254, 1270 to 1308, 1484 to 1564, 1578 to 1629, 1686 to 1705, 1748 to 1767, 1768 to 1787, 1794 to 1813, 1870 to 1913, 2015 to 2034, 2086 to 2118, 2129 to 2148, 2151 to 2184, 2206 to 2225, 2239 to 2272, 2287 to 2336, 2337 to 2373, 2387 to 2406, 2598 to 2757, 2775 to 2809, 2826 to 2866, 2959 to 2982, 3032 to 3051, 3052 to 3071, 3136 to 3155, 3168 to 3187, 3188 to 3207, 3232 to 3270, 3298 to 3344, 3408 to 3427, 3563 to 3582, 3590 to 3728, 3739 to 3769, 3877 to 3896, 3949 to 4030, and 4170 to 4225 of the base sequence of SEQ ID NO: 471.

In one embodiment, the antisense oligonucleotide of the present invention is complementary to a nucleic acid comprising at least 15 consecutive bases in a target region selected from the group consisting of positions 14 to 33, 34 to 53, 54 to 73, 148 to 167, 397 to 416, 398 to 417, 399 to 418, 400 to 419, 401 to 420, 402 to 421, 403 to 422, 404 to 423, 405 to 424, 406 to 425, 407 to 426, 408 to 427, 413 to 432, 723 to 742, 747 to 766, 756 to 775, 759 to 778, 965 to 984, 1127 to 1146, 1148 to 1167, 1151 to 1170, 1154 to 1173, 1182 to 1201, 1185 to 1204, 1188 to 1207, 1191 to 1210, 1194 to 1213, 1228 to 1247, 1231 to 1250, 1235 to 1254, 1270 to 1289, 1289 to 1308, 1484 to 1503, 1488 to 1507, 1491 to 1510, 1493 to 1512, 1506 to 1525, 1517 to 1536, 1531 to 1550, 1545 to 1564, 1578 to 1597, 1579 to 1598, 1580 to 1599, 1581 to 1600, 1582 to 1601, 1583 to 1602, 1584 to 1603, 1585 to 1604, 1586 to 1605, 1587 to 1606, 1588 to 1607, 1589 to 1608, 1590 to 1609, 1591 to 1610, 1592 to 1611, 1593 to 1612, 1594 to 1613, 1595 to 1614, 1596 to 1615, 1597 to 1616, 1598 to 1617, 1599 to 1618, 1600 to 1619, 1601 to 1620, 1602 to 1621, 1603 to 1622, 1604 to 1623, 1605 to 1624, 1607 to 1626, 1610 to 1629, 1686 to 1705, 1748 to 1767, 1768 to 1787, 1794 to 1813, 1870 to 1889, 1871 to 1890, 1874 to 1893, 1877 to 1896, 1878 to 1897, 1879 to 1898, 1880 to 1899, 1881 to 1900, 1882 to 1901, 1883 to 1902, 1884 to 1903, 1885 to 1904, 1886 to 1905, 1887 to 1906, 1889 to 1908, 1894 to 1913, 2015 to 2034, 2086 to 2105, 2093 to 2112, 2094 to 2113, 2097 to 2116, 2099 to 2118, 2129 to 2148, 2151 to 2170, 2165 to 2184, 2206 to 2225, 2239 to 2258, 2253 to 2272, 2287 to 2306, 2301 to 2320, 2317 to 2336, 2337 to 2356, 2354 to 2373, 2387 to 2406, 2598 to 2617, 2613 to 2632, 2614 to 2633, 2615 to 2634, 2616 to 2635, 2617 to 2636, 2618 to 2637, 2619 to 2638, 2620 to 2639, 2621 to 2640, 2622 to 2641, 2623 to 2642, 2624 to 2643, 2625 to 2644, 2626 to 2645, 2627 to 2646, 2628 to 2647, 2629 to 2648, 2630 to 2649, 2631 to 2650, 2632 to 2651, 2633 to 2652, 2634 to 2653, 2635 to 2654, 2636 to 2655, 2637 to 2656, 2638 to 2657, 2639 to 2658, 2640 to 2659, 2641 to 2660, 2642 to 2661, 2643 to 2662, 2644 to 2663, 2645 to 2664, 2646 to 2665, 2647 to 2666, 2648 to 2667, 2649 to 2668, 2663 to 2682, 2672 to 2691, 2673 to 2692, 2674 to 2693, 2675 to 2694, 2676 to 2695, 2677 to 2696, 2678 to 2697, 2679 to 2698, 2680 to 2699, 2681 to 2700, 2682 to 2701, 2683 to 2702, 2684 to 2703, 2685 to 2704, 2686 to 2705, 2687 to 2706, 2688 to 2707, 2689 to 2708, 2690 to 2709, 2691 to 2710, 2692 to 2711, 2693 to 2712, 2694 to 2713, 2695 to 2714, 2696 to 2715, 2697 to 2716, 2698 to 2717, 2699 to 2718, 2700 to 2719, 2701 to 2720, 2702 to 2721, 2703 to 2722, 2704 to 2723, 2705 to 2724, 2706 to 2725, 2707 to 2726, 2708 to 2727, 2709 to 2728, 2710 to 2729, 2711 to 2730, 2712 to 2731, 2713 to 2732, 2714 to 2733, 2715 to 2734, 2716 to 2735, 2717 to 2736, 2718 to 2737, 2719 to 2738, 2720 to 2739, 2721 to 2740, 2722 to 2741, 2723 to 2742, 2724 to 2743, 2725 to 2744, 2726 to 2745, 2727 to 2746, 2728 to 2747, 2729 to 2748, 2730 to 2749, 2731 to 2750, 2732 to 2751, 2733 to 2752, 2738 to 2757, 2775 to 2794, 2790 to 2809, 2826 to 2845, 2827 to 2846, 2828 to 2847, 2829 to 2848, 2830 to 2849, 2831 to 2850, 2832 to 2851, 2833 to 2852, 2834 to 2853, 2835 to 2854, 2836 to 2855, 2837 to 2856, 2838 to 2857, 2839 to 2858, 2840 to 2859, 2841 to 2860, 2842 to 2861, 2843 to 2862, 2844 to 2863, 2847 to 2866, 2959 to 2978, 2962 to 2981, 2963 to 2982, 3032 to 3051, 3052 to 3071, 3136 to 3155, 3168 to 3187, 3188 to 3207, 3232 to 3251, 3251 to 3270, 3298 to 3317, 3302 to 3321, 3303 to 3322, 3304 to 3323, 3305 to 3324, 3306 to 3325, 3307 to 3326, 3308 to 3327, 3309 to 3328, 3310 to 3329, 3311 to 3330, 3312 to 3331, 3313 to 3332, 3314 to 3333, 3315 to 3334, 3316 to 3335, 3317 to 3336, 3318 to 3337, 3319 to 3338, 3320 to 3339, 3321 to 3340, 3322 to 3341, 3323 to 3342, 3324 to 3343, 3325 to 3344, 3408 to 3427, 3563 to 3582, 3590 to 3609, 3608 to 3627, 3617 to 3636, 3618 to 3637, 3619 to 3638, 3620 to 3639, 3621 to 3640, 3622 to 3641, 3623 to 3642, 3624 to 3643, 3625 to 3644, 3626 to 3645, 3627 to 3646, 3628 to 3647, 3629 to 3648, 3630 to 3649, 3631 to 3650, 3632 to 3651, 3633 to 3652, 3634 to 3653, 3635 to 3654, 3636 to 3655, 3637 to 3656, 3638 to 3657, 3639 to 3658, 3640 to 3659, 3641 to 3660, 3642 to 3661, 3643 to 3662, 3644 to 3663, 3645 to 3664, 3646 to 3665, 3647 to 3666, 3648 to 3667, 3649 to 3668, 3650 to 3669, 3651 to 3670, 3652 to 3671, 3653 to 3672, 3654 to 3673, 3671 to 3690, 3672 to 3691, 3673 to 3692, 3674 to 3693, 3675 to 3694, 3676 to 3695, 3677 to 3696, 3678 to 3697, 3679 to 3698, 3680 to 3699, 3681 to 3700, 3682 to 3701, 3683 to 3702, 3684 to 3703, 3685 to 3704, 3686 to 3705, 3687 to 3706, 3688 to 3707, 3689 to 3708, 3690 to 3709, 3691 to 3710, 3692 to 3711, 3693 to 3712, 3694 to 3713, 3695 to 3714, 3696 to 3715, 3697 to 3716, 3698 to 3717, 3699 to 3718, 3700 to 3719, 3701 to 3720, 3702 to 3721, 3703 to 3722, 3704 to 3723, 3705 to 3724, 3706 to 3725, 3707 to 3726, 3708 to 3727, 3709 to 3728, 3739 to 3758, 3740 to 3759, 3741 to 3760, 3742 to 3761, 3743 to 3762, 3744 to 3763, 3745 to 3764, 3750 to 3769, 3877 to 3896, 3949 to 3968, 3964 to 3983, 3965 to 3984, 3966 to 3985, 3967 to 3986, 3968 to 3987, 3969 to 3988, 3970 to 3989, 3971 to 3990, 3972 to 3991, 3973 to 3992, 3974 to 3993, 3975 to 3994, 3976 to 3995, 3977 to 3996, 3978 to 3997, 3979 to 3998, 3980 to 3999, 3981 to 4000, 3982 to 4001, 3983 to 4002, 3984 to 4003, 3985 to 4004, 3986 to 4005, 3987 to 4006, 3988 to 4007, 3989 to 4008, 3990 to 4009, 3991 to 4010, 3992 to 4011, 3993 to 4012, 3994 to 4013, 3995 to 4014, 3996 to 4015, 3997 to 4016, 3998 to 4017, 3999 to 4018, 4011 to 4030, 4170 to 4189, 4173 to 4192, 4176 to 4195, 4177 to 4196, 4178 to 4197, 4179 to 4198, 4180 to 4199, 4181 to 4200, 4182 to 4201, 4183 to 4202, 4184 to 4203, 4185 to 4204, 4186 to 4205, 4187 to 4206, 4188 to 4207, 4189 to 4208, 4190 to 4209, 4191 to 4210, 4192 to 4211, 4193 to 4212, 4194 to 4213, 4195 to 4214, 4196 to 4215, 4197 to 4216, 4198 to 4217, 4199 to 4218, 4200 to 4219, 4201 to 4220, 4202 to 4221, 4203 to 4222, 4204 to 4223, 4205 to 4224, and 4206 to 4225 of the base sequence of SEQ ID NO: 471.

An example of the antisense oligonucleotide complementary to a nucleic acid comprising at least 15 consecutive bases in the target sequence includes an antisense oligonucleotide comprising or consisting of:
(i) a base sequence selected from the group consisting of SEQ ID NOs: 1 to 6, 8, 9, 11, 14 to 24, 26, 29, 31 to 60, 66 to 67, 73 to 75, 79 to 82, 86 to 89, 92 to 111, 115 to 136, 138 to 145, 147 to 158, and 160 to 464;
(ii) a base sequence having addition, deletion, or substitution of one or several base(s) in a base sequence selected from the group consisting of SEQ ID NOs: 1 to 6, 8, 9, 11, 14 to 24, 26, 29, 31 to 60, 66 to 67, 73 to 75, 79 to 82, 86 to 89, 92 to 111, 115 to 136, 138 to 145, 147 to 158, and 160 to 464; or
(iii) a base sequence having 90% or more sequence identity with a base sequence selected from the group consisting of SEQ ID NOs: 1 to 6, 8, 9, 11, 14 to 24, 26, 29, 31 to 60, 66 to 67, 73 to 75, 79 to 82, 86 to 89, 92 to 111, 115 to 136, 138 to 145, 147 to 158, and 160 to 464,
e.g., the base sequence (i).

As used herein, the term “several” used in the phrase of “base sequence having addition, deletion, or substitution of one or several base(s)” means, two, three, four, five, six, seven, eight, nine, or ten.

In one embodiment, the antisense oligonucleotide of the present invention is complementary to a nucleic acid comprising at least 15 consecutive bases in a target region selected from the group consisting of positions 14 to 33, 34 to 53, 398 to 427, 723 to 742, 747 to 766, 1148 to 1167, 1289 to 1308, 1484 to 1564, 1578 to 1624, 1686 to 1705, 1748 to 1767, 1768 to 1787, 1794 to 1813, 1877 to 1906, 2093 to 2113, 2151 to 2170, 2206 to 2225, 2239 to 2272, 2287 to 2336, 2337 to 2373, 2387 to 2406, 2613 to 2752, 2775 to 2809, 2826 to 2863, 2962 to 2982, 3032 to 3051, 3052 to 3071, 3168 to 3187, 3232 to 3270, 3302 to 3343, 3408 to 3427, 3590 to 3728, 3739 to 3764, 3877 to 3896, 3949 to 4030, and 4177 to 4225 of the base sequence of SEQ ID NO: 471.

In one embodiment, the antisense oligonucleotide of the present invention is complementary to a nucleic acid comprising at least 15 consecutive bases in a target region selected from the group consisting of positions 14 to 33, 34 to 53, 398 to 417, 399 to 418, 400 to 419, 401 to 420, 402 to 421, 403 to 422, 404 to 423, 405 to 424, 406 to 425, 407 to 426, 408 to 427, 723 to 742, 747 to 766, 1148 to 1167, 1289 to 1308, 1484 to 1503, 1488 to 1507, 1491 to 1510, 1493 to 1512, 1506 to 1525, 1517 to 1536, 1531 to 1550, 1545 to 1564, 1578 to 1597, 1579 to 1598, 1580 to 1599, 1581 to 1600, 1582 to 1601, 1583 to 1602, 1584 to 1603, 1585 to 1604, 1586 to 1605, 1587 to 1606, 1588 to 1607, 1589 to 1608, 1590 to 1609, 1591 to 1610, 1592 to 1611, 1593 to 1612, 1594 to 1613, 1595 to 1614, 1596 to 1615, 1597 to 1616, 1598 to 1617, 1599 to 1618, 1600 to 1619, 1601 to 1620, 1602 to 1621, 1603 to 1622, 1604 to 1623, 1605 to 1624, 1686 to 1705, 1748 to 1767, 1768 to 1787, 1794 to 1813, 1877 to 1896, 1878 to 1897, 1879 to 1898, 1880 to 1899, 1881 to 1900, 1882 to 1901, 1883 to 1902, 1884 to 1903, 1885 to 1904, 1886 to 1905, 1887 to 1906, 2093 to 2112, 2094 to 2113, 2151 to 2170, 2206 to 2225, 2239 to 2258, 2253 to 2272, 2287 to 2306, 2301 to 2320, 2317 to 2336, 2337 to 2356, 2354 to 2373, 2387 to 2406, 2613 to 2632, 2614 to 2633, 2615 to 2634, 2616 to 2635, 2617 to 2636, 2618 to 2637, 2619 to 2638, 2620 to 2639, 2621 to 2640, 2622 to 2641, 2623 to 2642, 2624 to 2643, 2625 to 2644, 2626 to 2645, 2627 to 2646, 2628 to 2647, 2629 to 2648, 2630 to 2649, 2631 to 2650, 2632 to 2651, 2633 to 2652, 2634 to 2653, 2635 to 2654, 2636 to 2655, 2637 to 2656, 2638 to 2657, 2639 to 2658, 2640 to 2659, 2641 to 2660, 2642 to 2661, 2643 to 2662, 2644 to 2663, 2645 to 2664, 2646 to 2665, 2647 to 2666, 2648 to 2667, 2649 to 2668, 2663 to 2682, 2672 to 2691, 2673 to 2692, 2674 to 2693, 2675 to 2694, 2676 to 2695, 2677 to 2696, 2678 to 2697, 2679 to 2698, 2680 to 2699, 2681 to 2700, 2682 to 2701, 2683 to 2702, 2684 to 2703, 2685 to 2704, 2686 to 2705, 2687 to 2706, 2688 to 2707, 2689 to 2708, 2690 to 2709, 2691 to 2710, 2692 to 2711, 2693 to 2712, 2694 to 2713, 2695 to 2714, 2696 to 2715, 2697 to 2716, 2698 to 2717, 2699 to 2718, 2700 to 2719, 2701 to 2720, 2702 to 2721, 2703 to 2722, 2704 to 2723, 2705 to 2724, 2706 to 2725, 2707 to 2726, 2708 to 2727, 2709 to 2728, 2710 to 2729, 2711 to 2730, 2712 to 2731, 2713 to 2732, 2714 to 2733, 2715 to 2734, 2716 to 2735, 2717 to 2736, 2718 to 2737, 2719 to 2738, 2720 to 2739, 2721 to 2740, 2722 to 2741, 2723 to 2742, 2724 to 2743, 2725 to 2744, 2726 to 2745, 2727 to 2746, 2728 to 2747, 2729 to 2748, 2730 to 2749, 2731 to 2750, 2732 to 2751, 2733 to 2752, 2775 to 2794, 2790 to 2809, 2826 to 2845, 2827 to 2846, 2828 to 2847, 2829 to 2848, 2830 to 2849, 2831 to 2850, 2832 to 2851, 2833 to 2852, 2834 to 2853, 2835 to 2854, 2836 to 2855, 2837 to 2856, 2838 to 2857, 2839 to 2858, 2840 to 2859, 2841 to 2860, 2842 to 2861, 2843 to 2862, 2844 to 2863, 2962 to 2981, 2963 to 2982, 3032 to 3051, 3052 to 3071, 3168 to 3187, 3232 to 3251, 3251 to 3270, 3302 to 3321, 3303 to 3322, 3304 to 3323, 3305 to 3324, 3306 to 3325, 3307 to 3326, 3308 to 3327, 3309 to 3328, 3310 to 3329, 3311 to 3330, 3312 to 3331, 3313 to 3332, 3314 to 3333, 3315 to 3334, 3316 to 3335, 3317 to 3336, 3318 to 3337, 3319 to 3338, 3320 to 3339, 3321 to 3340, 3322 to 3341, 3323 to 3342, 3324 to 3343, 3408 to 3427, 3590 to 3609, 3608 to 3627, 3617 to 3636, 3618 to 3637, 3619 to 3638, 3620 to 3639, 3621 to 3640, 3622 to 3641, 3623 to 3642, 3624 to 3643, 3625 to 3644, 3626 to 3645, 3627 to 3646, 3628 to 3647, 3629 to 3648, 3630 to 3649, 3631 to 3650, 3632 to 3651, 3633 to 3652, 3634 to 3653, 3635 to 3654, 3636 to 3655, 3637 to 3656, 3638 to 3657, 3639 to 3658, 3640 to 3659, 3641 to 3660, 3642 to 3661, 3643 to 3662, 3644 to 3663, 3645 to 3664, 3646 to 3665, 3647 to 3666, 3648 to 3667, 3649 to 3668, 3650 to 3669, 3651 to 3670, 3652 to 3671, 3653 to 3672, 3654 to 3673, 3671 to 3690, 3672 to 3691, 3673 to 3692, 3674 to 3693, 3675 to 3694, 3676 to 3695, 3677 to 3696, 3678 to 3697, 3679 to 3698, 3680 to 3699, 3681 to 3700, 3682 to 3701, 3683 to 3702, 3684 to 3703, 3685 to 3704, 3686 to 3705, 3687 to 3706, 3688 to 3707, 3689 to 3708, 3690 to 3709, 3691 to 3710, 3692 to 3711, 3693 to 3712, 3694 to 3713, 3695 to 3714, 3696 to 3715, 3697 to 3716, 3698 to 3717, 3699 to 3718, 3700 to 3719, 3701 to 3720, 3702 to 3721, 3703 to 3722, 3704 to 3723, 3705 to 3724, 3706 to 3725, 3707 to 3726, 3708 to 3727, 3709 to 3728, 3739 to 3758, 3740 to 3759, 3741 to 3760, 3742 to 3761, 3743 to 3762, 3744 to 3763, 3745 to 3764, 3877 to 3896, 3949 to 3968, 3964 to 3983, 3965 to 3984, 3966 to 3985, 3967 to 3986, 3968 to 3987, 3969 to 3988, 3970 to 3989, 3971 to 3990, 3972 to 3991, 3973 to 3992, 3974 to 3993, 3975 to 3994, 3976 to 3995, 3977 to 3996, 3978 to 3997, 3979 to 3998, 3980 to 3999, 3981 to 4000, 3982 to 4001, 3983 to 4002, 3984 to 4003, 3985 to 4004, 3986 to 4005, 3987 to 4006, 3988 to 4007, 3989 to 4008, 3990 to 4009, 3991 to 4010, 3992 to 4011, 3993 to 4012, 3994 to 4013, 3995 to 4014, 3996 to 4015, 3997 to 4016, 3998 to 4017, 3999 to 4018, 4011 to 4030, 4177 to 4196, 4178 to 4197, 4179 to 4198, 4180 to 4199, 4181 to 4200, 4182 to 4201, 4183 to 4202, 4184 to 4203, 4185 to 4204, 4186 to 4205, 4187 to 4206, 4188 to 4207, 4189 to 4208, 4190 to 4209, 4191 to 4210, 4192 to 4211, 4193 to 4212, 4194 to 4213, 4195 to 4214, 4196 to 4215, 4197 to 4216, 4198 to 4217, 4199 to 4218, 4200 to 4219, 4201 to 4220, 4202 to 4221, 4203 to 4222, 4204 to 4223, 4205 to 4224, and 4206 to 4225 of the base sequence of SEQ ID NO: 471.

An example of the antisense oligonucleotide complementary to a nucleic acid comprising at least 15 consecutive bases in the target sequence includes an antisense oligonucleotide comprising or consisting of:
(i) a base sequence selected from the group consisting of SEQ ID NOs: 1, 5, 6, 14 to 24, 31 to 35, 37 to 40, 42 to 45, 47 to 50, 52 to 59, 73, 92 to 100, 103, 107 to 110, 120 to 136, 138 to 143, 150 to 153, 155 to 158, and 163 to 464;
(ii) a base sequence having addition, deletion, or substitution of one or several base(s) in a base sequence selected from the group consisting of SEQ ID NOs: 1, 5, 6, 14 to 24, 31 to 35, 37 to 40, 42 to 45, 47 to 50, 52 to 59, 73, 92 to 100, 103, 107 to 110, 120 to 136, 138 to 143, 150 to 153, 155 to 158, and 163 to 464; or
(iii) a base sequence having 90% or more sequence identity with a base sequence selected from the group consisting of SEQ ID NOs: 1, 5, 6, 14 to 24, 31 to 35, 37 to 40, 42 to 45, 47 to 50, 52 to 59, 73, 92 to 100, 103, 107 to 110, 120 to 136, 138 to 143, 150 to 153, 155 to 158, and 163 to 464,
e.g., the base sequence (i).

In one embodiment, the antisense oligonucleotide of the present invention is complementary to a nucleic acid comprising at least 15 consecutive bases in a target region selected from the group consisting of positions 14 to 33, 34 to 53, 54 to 73, 148 to 167, 397 to 432, 747 to 778, 965 to 984, 1127 to 1146, 1148 to 1173, 1182 to 1213, 1228 to 1254, 1270 to 1308, 1484 to 1564, 1578 to 1629, 1686 to 1705, 1748 to 1767, 1768 to 1787, 1794 to 1813, 1870 to 1913, 2015 to 2034, 2086 to 2118, 2129 to 2148, 2151 to 2170, 2206 to 2225, 2239 to 2272, 2287 to 2336, 2337 to 2373, 2387 to 2406, 2598 to 2632, 2633 to 2668, 2672 to 2691, 2699 to 2757, 2775 to 2809, 2826 to 2866, 2959 to 2978, 3032 to 3051, 3052 to 3071, 3136 to 3155, 3168 to 3187, 3188 to 3207, 3232 to 3270, 3298 to 3317, 3325 to 3344, 3408 to 3427, 3590 to 3690, 3691 to 3728, 3739 to 3769, 3877 to 3896, 3949 to 3983, 3984 to 4030, and 4170 to 4198 of the base sequence of SEQ ID NO: 471.

In one embodiment, the antisense oligonucleotide of the present invention is complementary to a nucleic acid comprising at least 15 consecutive bases in a target region selected from the group consisting of positions 14 to 33, 34 to 53, 54 to 73, 148 to 167, 397 to 416, 413 to 432, 747 to 766, 756 to 775, 759 to 778, 965 to 984, 1127 to 1146, 1148 to 1167, 1151 to 1170, 1154 to 1173, 1182 to 1201, 1185 to 1204, 1188 to 1207, 1191 to 1210, 1194 to 1213, 1228 to 1247, 1231 to 1250, 1235 to 1254, 1270 to 1289, 1289 to 1308, 1484 to 1503, 1488 to 1507, 1491 to 1510, 1493 to 1512, 1506 to 1525, 1517 to 1536, 1531 to 1550, 1545 to 1564, 1578 to 1597, 1586 to 1605, 1589 to 1608, 1592 to 1611, 1595 to 1614, 1598 to 1617, 1601 to 1620, 1604 to 1623, 1607 to 1626, 1610 to 1629, 1686 to 1705, 1748 to 1767, 1768 to 1787, 1794 to 1813, 1870 to 1889, 1871 to 1890, 1874 to 1893, 1877 to 1896, 1880 to 1899, 1883 to 1902, 1886 to 1905, 1889 to 1908, 1894 to 1913, 2015 to 2034, 2086 to 2105, 2097 to 2116, 2099 to 2118, 2129 to 2148, 2151 to 2170, 2206 to 2225, 2239 to 2258, 2253 to 2272, 2287 to 2306, 2301 to 2320, 2317 to 2336, 2337 to 2356, 2354 to 2373, 2387 to 2406, 2598 to 2617, 2613 to 2632, 2633 to 2652, 2649 to 2668, 2672 to 2691, 2699 to 2718, 2702 to 2721, 2705 to 2724, 2708 to 2727, 2711 to 2730, 2714 to 2733, 2717 to 2736, 2720 to 2739, 2723 to 2742, 2726 to 2745, 2729 to 2748, 2738 to 2757, 2775 to 2794, 2790 to 2809, 2826 to 2845, 2829 to 2848, 2832 to 2851, 2835 to 2854, 2838 to 2857, 2841 to 2860, 2847 to 2866, 2959 to 2978, 3032 to 3051, 3052 to 3071, 3136 to 3155, 3168 to 3187, 3188 to 3207, 3232 to 3251, 3251 to 3270, 3298 to 3317, 3325 to 3344, 3408 to 3427, 3590 to 3609, 3608 to 3627, 3617 to 3636, 3635 to 3654, 3654 to 3673, 3671 to 3690, 3691 to 3710, 3709 to 3728, 3739 to 3758, 3750 to 3769, 3877 to 3896, 3949 to 3968, 3964 to 3983, 3984 to 4003, 3999 to 4018, 4011 to 4030, 4170 to 4189, 4173 to 4192, 4176 to 4195, and 4179 to 4198 of the base sequence of SEQ ID NO: 471.

An example of the antisense oligonucleotide complementary to a nucleic acid comprising at least 15 consecutive bases in the target sequence includes an antisense oligonucleotide comprising or consisting of:
(i) a base sequence selected from the group consisting of SEQ ID NOs: 1 to 4, 6, 8, 9, 11, 14 to 24, 26, 29, 31 to 37, 39 to 50, 52 to 60, 66 to 67, 73 to 75, 79 to 82, 86 to 89, 92 to 111, 115 to 118, 120 to 136, 138 to 145, 147 to 158, and 160 to 163;
(ii) a base sequence having addition, deletion, or substitution of one or several base(s) in a base sequence selected from the group consisting of SEQ ID NOs: 1 to 4, 6, 8, 9, 11, 14 to 24, 26, 29, 31 to 37, 39 to 50, 52 to 60, 66 to 67, 73 to 75, 79 to 82, 86 to 89, 92 to 111, 115 to 118, 120 to 136, 138 to 145, 147 to 158, and 160 to 163; or
(iii) a base sequence having 90% or more sequence identity with a base sequence selected from the group consisting of SEQ ID NOs: 1 to 4, 6, 8, 9, 11, 14 to 24, 26, 29, 31 to 37, 39 to 50, 52 to 60, 66 to 67, 73 to 75, 79 to 82, 86 to 89, 92 to 111, 115 to 118, 120 to 136, 138 to 145, 147 to 158, and 160 to 163,
e.g., the base sequence (i).

In one embodiment, the antisense oligonucleotide of the present invention is complementary to a nucleic acid comprising at least 15 consecutive bases in a target region selected from the group consisting of positions 14 to 33, 34 to 53, 747 to 766, 1148 to 1167, 1289 to 1308, 1484 to 1564, 1578 to 1623, 1686 to 1705, 1748 to 1767, 1768 to 1787, 1794 to 1813, 1877 to 1905, 2151 to 2170, 2206 to 2225, 2239 to 2272, 2287 to 2336, 2337 to 2373, 2387 to 2406, 2613 to 2632, 2633 to 2668, 2672 to 2691, 2699 to 2748, 2775 to 2809, 2826 to 2860, 3032 to 3051, 3052 to 3071, 3168 to 3187, 3232 to 3270, 3408 to 3427, 3590 to 3690, 3691 to 3728, 3739 to 3758, 3877 to 3896, 3949 to 3983, 3984 to 4030, and 4179 to 4198 of the base sequence of SEQ ID NO: 471.

In one embodiment, the antisense oligonucleotide of the present invention is complementary to a nucleic acid comprising at least 15 consecutive bases in a target region selected from the group consisting of positions 14 to 33, 34 to 53, 747 to 766, 1148 to 1167, 1289 to 1308, 1484 to 1503, 1488 to 1507, 1491 to 1510, 1493 to 1512, 1506 to 1525, 1517 to 1536, 1531 to 1550, 1545 to 1564, 1578 to 1597, 1586 to 1605, 1589 to 1608, 1592 to 1611, 1595 to 1614, 1598 to 1617, 1601 to 1620, 1604 to 1623, 1686 to 1705, 1748 to 1767, 1768 to 1787, 1794 to 1813, 1877 to 1896, 1880 to 1899, 1883 to 1902, 1886 to 1905, 2151 to 2170, 2206 to 2225, 2239 to 2258, 2253 to 2272, 2287 to 2306, 2301 to 2320, 2317 to 2336, 2337 to 2356, 2354 to 2373, 2387 to 2406, 2613 to 2632, 2633 to 2652, 2649 to 2668, 2672 to 2691, 2699 to 2718, 2702 to 2721, 2705 to 2724, 2708 to 2727, 2711 to 2730, 2714 to 2733, 2717 to 2736, 2720 to 2739, 2723 to 2742, 2726 to 2745, 2729 to 2748, 2775 to 2794, 2790 to 2809, 2826 to 2845, 2829 to 2848, 2832 to 2851, 2835 to 2854, 2838 to 2857, 2841 to 2860, 3032 to 3051, 3052 to 3071, 3168 to 3187, 3232 to 3251, 3251 to 3270, 3408 to 3427, 3590 to 3609, 3608 to 3627, 3617 to 3636, 3635 to 3654, 3654 to 3673, 3671 to 3690, 3691 to 3710, 3709 to 3728, 3739 to 3758, 3877 to 3896, 3949 to 3968, 3964 to 3983, 3984 to 4003, 3999 to 4018, 4011 to 4030, and 4179 to 4198 of the base sequence of SEQ ID NO: 471.

An example of the antisense oligonucleotide complementary to a nucleic acid comprising at least 15 consecutive bases in the target sequence includes an antisense oligonucleotide comprising or consisting of:
(i) a base sequence selected from the group consisting of SEQ ID NOs: 1, 6, 14 to 24, 31 to 35, 37, 39, 40, 42 to 45, 47 to 50, 52 to 59, 73, 92 to 100, 103, 107 to 110, 120 to 136, 138 to 143, 150 to 153, 155 to 158, and 163;
(ii) a base sequence having addition, deletion, or substitution of one or several base(s) in a base sequence selected from the group consisting of SEQ ID NOs: 1, 6, 14 to 24, 31 to 35, 37, 39, 40, 42 to 45, 47 to 50, 52 to 59, 73, 92 to 100, 103, 107 to 110, 120 to 136, 138 to 143, 150 to 153, 155 to 158, and 163; or
(iii) a base sequence having 90% or more sequence identity with a base sequence selected from the group consisting of SEQ ID NOs: 1, 6, 14 to 24, 31 to 35, 37, 39, 40, 42 to 45, 47 to 50, 52 to 59, 73, 92 to 100, 103, 107 to 110, 120 to 136, 138 to 143, 150 to 153, 155 to 158, and 163,
e.g., the base sequence (i).

In one embodiment, the antisense oligonucleotide of the present invention is selected from those having a ratio (average value) of the ATN-1 gene expression level obtained by administrating the antisense oligonucleotide to A204 cells to the ATN-1 gene expression level obtained by administrating OMe-6 or NRH-71, a positive control antisense oligonucleotide to A204 cells, of 1.0 or less in any of Examples, as measured by a method described in Examples of the present specification. Examples of such antisense oligonucleotides include antisense oligonucleotides consisting of 15 to 22 nucleotides complementary to a nucleic acid comprising at least 15 consecutive bases in a target region selected from the group consisting of positions 397 to 416, 759 to 778, 1127 to 1146, 1148 to 1173, 1182 to 1213, 1488 to 1525, 1586 to 1629, 1686 to 1705, 1768 to 1787, 1870 to 1908, 2097 to 2118, 2151 to 2170, 2239 to 2258, 2287 to 2306, 2317 to 2336, 2633 to 2652, 2702 to 2745, 2832 to 2866, 2959 to 2978, 3298 to 3317, 3635 to 3654, 3671 to 3690, 3691 to 3710, 3750 to 3983, 3984 to 4003, and 4170 to 4198 of the base sequence of SEQ ID NO: 471. Other examples include antisense oligonucleotides consisting of 15 to 22 nucleotides complementary to a nucleic acid comprising at least 15 consecutive bases in a target region selected from the group consisting of positions 397 to 416, 759 to 778, 1127 to 1146, 1148 to 1167, 1151 to 1170, 1154 to 1173, 1182 to 1201, 1185 to 1204, 1194 to 1213, 1488 to 1507, 1491 to 1510, 1506 to 1525, 1586 to 1605, 1589 to 1608, 1595 to 1614, 1598 to 1617, 1601 to 1620, 1604 to 1623, 1607 to 1626, 1610 to 1629, 1686 to 1705, 1768 to 1787, 1870 to 1889, 1871 to 1890, 1874 to 1893, 1877 to 1896, 1886 to 1905, 1889 to 1908, 2097 to 2116, 2099 to 2118, 2151 to 2170, 2239 to 2258, 2287 to 2306, 2317 to 2336, 2633 to 2652, 2702 to 2721, 2705 to 2724, 2708 to 2727, 2711 to 2730, 2714 to 2733, 2717 to 2736, 2720 to 2739, 2726 to 2745, 2832 to 2851, 2835 to 2854, 2841 to 2860, 2847 to 2866, 2959 to 2978, 3298 to 3317, 3635 to 3654, 3671 to 3690, 3691 to 3710, 3750 to 3769, 3964 to 3983, 3984 to 4003, 4170 to 4189, 4173 to 4192, 4176 to 4195, and 4179 to 4198 of the base sequence of SEQ ID NO: 471.

An example of the antisense oligonucleotide complementary to a nucleic acid comprising at least 15 consecutive bases in the target sequence includes an antisense oligonucleotide comprising or consisting of:
(i) a base sequence selected from the group consisting of SEQ ID NOs: 9, 11, 16, 17, 22, 31, 60, 67, 73, 74, 75, 79, 80, 92, 95 to 107, 110, 111, 116, 117, 120 to 122, 126, 128 to 134, 136, 140, 141, 143 to 145, 148, 151 to 154, 156, 157, and 160 to 163;
(ii) a base sequence having addition, deletion, or substitution of one or several base(s) in a base sequence selected from the group consisting of SEQ ID NOs: 9, 11, 16, 17, 22, 31, 60, 67, 73, 74, 75, 79, 80, 92, 95 to 107, 110, 111, 116, 117, 120 to 122, 126, 128 to 134, 136, 140, 141, 143 to 145, 148, 151 to 154, 156, 157, and 160 to 163; or
(iii) a base sequence having 90% or more sequence identity with a base sequence selected from the group consisting of SEQ ID NOs: 9, 11, 16, 17, 22, 31, 60, 67, 73, 74, 75, 79, 80, 92, 95 to 107, 110, 111, 116, 117, 120 to 122, 126, 128 to 134, 136, 140, 141, 143 to 145, 148, 151 to 154, 156, 157, and 160 to 163,
e.g., the base sequence (i).

In one embodiment, the antisense oligonucleotide of the present invention is selected from those having a ratio (average value) of the ATN-1 gene expression level obtained by administrating the antisense oligonucleotide to A204 cells to the ATN-1 gene expression level obtained by administrating OMe-6 or NRH-71, a positive control antisense oligonucleotide to A204 cells, of 0.75 or less in any of Examples, as measured by a method described in Examples of the present specification. Examples of such antisense oligonucleotides include antisense oligonucleotides consisting of 15 to 22 nucleotides complementary to a nucleic acid comprising at least 15 consecutive bases in a target region selected from the group consisting of positions 397 to 416, 1194 to 1213, 1506 to 1525, 1586 to 1626, 1870 to 1896, 2097 to 2116, 2317 to 2336, 2633 to 2652, 2702 to 2739, 2841 to 2978, 3298 to 3317, 3635 to 3654, 3691 to 3710, 3750 to 3769, 3984 to 4003, and 4170 to 4198 of the base sequence of SEQ ID NO: 471. Other examples include antisense oligonucleotides consisting of 15 to 22 nucleotides complementary to a nucleic acid comprising at least 15 consecutive bases in a target region selected from the group consisting of positions 397 to 416, 1194 to 1213, 1506 to 1525, 1586 to 1605, 1595 to 1614, 1598 to 1617, 1604 to 1623, 1607 to 1626, 1870 to 1889, 1871 to 1890, 1874 to 1893, 1877 to 1896, 2097 to 2116, 2317 to 2336, 2633 to 2652, 2702 to 2721, 2708 to 2727, 2711 to 2730, 2714 to 2733, 2717 to 2736, 2720 to 2739, 2841 to 2860, 2959 to 2978, 3298 to 3317, 3635 to 3654, 3691 to 3710, 3750 to 3769, 3984 to 4003, 4170 to 4189, 4173 to 4192, 4176 to 4195, and 4179 to 4198 of the base sequence of SEQ ID NO: 471.

An example of the antisense oligonucleotide complementary to a nucleic acid comprising at least 15 consecutive bases in the target sequence includes an antisense oligonucleotide comprising or consisting of:
(i) a base sequence selected from the group consisting of SEQ ID NOs: 11, 60, 92, 95, 97, 98, 100, 101, 104 to 107, 116, 122, 126, 128, 130 to 134, 143, 145, 148, 151, 153, 154, 157, and 160 to 163;
(ii) a base sequence having addition, deletion, or substitution of one or several base(s) in a base sequence selected from the group consisting of SEQ ID NOs: 11, 60, 92, 95, 97, 98, 100, 101, 104 to 107, 116, 122, 126, 128, 130 to 134, 143, 145, 148, 151, 153, 154, 157, and 160 to 163; or
(iii) a base sequence having 90% or more sequence identity with a base sequence selected from the group consisting of SEQ ID NOs: 11, 60, 92, 95, 97, 98, 100, 101, 104 to 107, 116, 122, 126, 128, 130 to 134, 143, 145, 148, 151, 153, 154, 157, and 160 to 163,
e.g., the base sequence (i).

In one embodiment, the antisense oligonucleotide of the present invention is selected from those having a ratio (average value) of the ATN-1 gene expression level obtained by administrating the antisense oligonucleotide to A204 cells to the ATN-1 gene expression level obtained by administrating OMe-6 or NRH-71, a positive control antisense oligonucleotide to A204 cells, of 0.5 or less in any of Examples, as measured by a method described in Examples of the present specification. Examples of such antisense oligonucleotides include antisense oligonucleotides consisting of 15 to 22 nucleotides complementary to a nucleic acid comprising at least 15 consecutive bases in a target region selected from the group consisting of positions 1598 to 1626, 1870 to 1890, 2097 to 2116, 2711 to 2736, 2841 to 2860, and 4170 to 4198 of the base sequence of SEQ ID NO: 471. Other examples include antisense oligonucleotides consisting of 15 to 22 nucleotides complementary to a nucleic acid comprising at least 15 consecutive bases in a target region selected from the group consisting of positions 1598 to 1617, 1604 to 1623, 1607 to 1626, 1870 to 1889, 1871 to 1890, 2097 to 2116, 2711 to 2730, 2714 to 2733, 2717 to 2736, 2841 to 2860, 4170 to 4189, 4173 to 4192, and 4179 to 4198 of the base sequence of SEQ ID NO: 471.

An example of the antisense oligonucleotide complementary to a nucleic acid comprising at least 15 consecutive bases in the target sequence includes an antisense oligonucleotide comprising or consisting of:
(i) a base sequence selected from the group consisting of SEQ ID NOs: 98, 100, 101, 104, 105, 116, 131 to 133, 143, 160, 161, and 163;
(ii) a base sequence having addition, deletion, or substitution of one or several base(s) in a base sequence selected from the group consisting of SEQ ID NOs: 98, 100, 101, 104, 105, 116, 131 to 133, 143, 160, 161, and 163; or
(iii) a base sequence having 90% or more sequence identity with a base sequence selected from the group consisting of SEQ ID NOs: 98, 100, 101, 104, 105, 116, 131 to 133, 143, 160, 161, and 163,
e.g., the base sequence (i).

In one embodiment, the antisense oligonucleotide of the present invention is complementary to a nucleic acid comprising at least 15 consecutive bases in a target region selected from the group consisting of positions 400 to 425, 1581 to 1624, 1878 to 1898, 2094 to 2113, 2629 to 2656, 2700 to 2741, 2839 to 2863, 2962 to 2981, 3302 to 3325, 3631 to 3658, 3687 to 3714, 3745 to 3764, 3980 to 4007, 4177 to 4199, and 4204 to 4223 of the base sequence of SEQ ID NO: 471.

In one embodiment, the antisense oligonucleotide of the present invention is complementary to a nucleic acid comprising at least 15 consecutive bases in a target region selected from the group consisting of positions 400 to 419, 403 to 422, 406 to 425, 1581 to 1600, 1582 to 1601, 1583 to 1602, 1584 to 1603, 1585 to 1604, 1587 to 1606, 1588 to 1607, 1590 to 1609, 1591 to 1610, 1592 to 1611, 1593 to 1612, 1594 to 1613, 1596 to 1615, 1597 to 1616, 1599 to 1618, 1600 to 1619, 1602 to 1621, 1603 to 1622, 1605 to 1624, 1878 to 1897, 1879 to 1898, 2094 to 2113, 2629 to 2648, 2637 to 2656, 2700 to 2719, 2701 to 2720, 2703 to 2722, 2704 to 2723, 2706 to 2725, 2707 to 2726, 2709 to 2728, 2710 to 2729, 2712 to 2731, 2713 to 2732, 2715 to 2734, 2716 to 2735, 2718 to 2737, 2719 to 2738, 2721 to 2740, 2722 to 2741, 2839 to 2858, 2840 to 2859, 2842 to 2861, 2843 to 2862, 2844 to 2863, 2962 to 2981, 3302 to 3321, 3306 to 3325, 3631 to 3650, 3632 to 3651, 3633 to 3652, 3634 to 3653, 3636 to 3655, 3637 to 3656, 3638 to 3657, 3639 to 3658, 3687 to 3706, 3695 to 3714, 3745 to 3764, 3980 to 3999, 3988 to 4007, 4177 to 4196, 4178 to 4197, 4180 to 4199, and 4204 to 4223 of the base sequence of SEQ ID NO: 471.

An example of the antisense oligonucleotide complementary to a nucleic acid comprising at least 15 consecutive bases in the target sequence includes an antisense oligonucleotide comprising or consisting of:
(i) a base sequence selected from the group consisting of SEQ ID NOs: 21, 164 to 222, 233, 234, and 350 to 355;
(ii) a base sequence having addition, deletion, or substitution of one or several base(s) in a base sequence selected from the group consisting of SEQ ID NOs: 21, 164 to 222, 233, 234, and 350 to 355; or
(iii) a base sequence having 90% or more sequence identity with a base sequence selected from the group consisting of SEQ ID NOs: 21, 164 to 222, 233, 234, and 350 to 355,
e.g., the base sequence (i).

In one embodiment, the antisense oligonucleotide of the present invention is selected from those having a ratio (average value) of the ATN-1 gene expression level obtained by administrating the antisense oligonucleotide to A204 cells to the ATN-1 gene expression level obtained by administrating OMe-6 or NRH-71, a positive control antisense oligonucleotide to A204 cells, of 1.0 or less in any of Examples, as measured by a method described in Examples of the present specification. Examples of such antisense oligonucleotides include antisense oligonucleotides consisting of 15 to 22 nucleotides complementary to a nucleic acid comprising at least 15 consecutive bases in a target region selected from the group consisting of positions 400 to 425, 1581 to 1624, 1878 to 1898, 2637 to 2656, 2700 to 2741, 2839 to 2863, 2962 to 2981, 3302 to 3325, 3631 to 3658, 3687 to 3706, 3745 to 3764, 4177 to 4199, and 4204 to 4223 of the base sequence of SEQ ID NO: 471. Other examples include antisense oligonucleotides consisting of 15 to 22 nucleotides complementary to a nucleic acid comprising at least 15 consecutive bases in a target region selected from the group consisting of positions 400 to 419, 403 to 422, 406 to 425, 1581 to 1600, 1582 to 1601, 1583 to 1602, 1584 to 1603, 1585 to 1604, 1587 to 1606, 1588 to 1607, 1591 to 1610, 1592 to 1611, 1593 to 1612, 1594 to 1613, 1596 to 1615, 1597 to 1616, 1600 to 1619, 1602 to 1621, 1603 to 1622, 1605 to 1624, 1878 to 1897, 1879 to 1898, 2637 to 2656, 2700 to 2719, 2701 to 2720, 2703 to 2722, 2704 to 2723, 2706 to 2725, 2707 to 2726, 2709 to 2728, 2710 to 2729, 2712 to 2731, 2713 to 2732, 2715 to 2734, 2716 to 2735, 2718 to 2737, 2719 to 2738, 2721 to 2740, 2722 to 2741, 2839 to 2858, 2840 to 2859, 2842 to 2861, 2843 to 2862, 2844 to 2863, 2962 to 2981, 3302 to 3321, 3306 to 3325, 3631 to 3650, 3632 to 3651, 3633 to 3652, 3634 to 3653, 3636 to 3655, 3637 to 3656, 3638 to 3657, 3639 to 3658, 3687 to 3706, 3745 to 3764, 4177 to 4196, 4178 to 4197, 4180 to 4199, and 4204 to 4223 of the base sequence of SEQ ID NO: 471.

An example of the antisense oligonucleotide complementary to a nucleic acid comprising at least 15 consecutive bases in the target sequence includes an antisense oligonucleotide comprising or consisting of:
(i) a base sequence selected from the group consisting of SEQ ID NOs: 21, 164 to 171, 173 to 177, 179 to 184, 187 to 214, 216, 219 to 222, 233 to 234, and 350 to 355;
(ii) a base sequence having addition, deletion, or substitution of one or several base(s) in a base sequence selected from the group consisting of SEQ ID NOs: 21, 164 to 171, 173 to 177, 179 to 184, 187 to 214, 216, 219 to 222, 233 to 234, and 350 to 355; or
(iii) a base sequence having 90% or more sequence identity with a base sequence selected from the group consisting of SEQ ID NOs: 21, 164 to 171, 173 to 177, 179 to 184, 187 to 214, 216, 219 to 222, 233 to 234, and 350 to 355,
e.g., the base sequence (i).

In one embodiment, the antisense oligonucleotide of the present invention is selected from those having a ratio (average value) of the ATN-1 gene expression level obtained by administrating the antisense oligonucleotide to A204 cells to the ATN-1 gene expression level obtained by administrating OMe-6 or NRH-71, a positive control antisense oligonucleotide to A204 cells, of 0.75 or less in any of Examples, as measured by a method described in Examples of the present specification. Examples of such antisense oligonucleotides include antisense oligonucleotides consisting of 15 to 22 nucleotides complementary to a nucleic acid comprising at least 15 consecutive bases in a target region selected from the group consisting of positions 1581 to 1624, 2703 to 2740, 3633 to 3655, and 4177 to 4199 of the base sequence of SEQ ID NO: 471. Other examples include antisense oligonucleotides consisting of 15 to 22 nucleotides complementary to a nucleic acid comprising at least 15 consecutive bases in a target region selected from the group consisting of positions 1581 to 1600, 1582 to 1601, 1584 to 1603, 1585 to 1604, 1587 to 1606, 1588 to 1607, 1594 to 1613, 1596 to 1615, 1597 to 1616, 1602 to 1621, 1603 to 1622, 1605 to 1624, 2703 to 2722, 2707 to 2726, 2709 to 2728, 2710 to 2729, 2712 to 2731, 2713 to 2732, 2715 to 2734, 2716 to 2735, 2718 to 2737, 2719 to 2738, 2721 to 2740, 3633 to 3652, 3634 to 3653, 3636 to 3655, 4177 to 4196, 4178 to 4197, and 4180 to 4199 of the base sequence of SEQ ID NO: 471.

An example of the antisense oligonucleotide complementary to a nucleic acid comprising at least 15 consecutive bases in the target sequence includes an antisense oligonucleotide comprising or consisting of:
(i) a base sequence selected from the group consisting of SEQ ID NOs: 167 to 171, 175 to 177, 180 to 182, 190, 193 to 202, 219 to 221, 233, and 351 to 353;
(ii) a base sequence having addition, deletion, or substitution of one or several base(s) in a base sequence selected from the group consisting of SEQ ID NOs: 167 to 171, 175 to 177, 180 to 182, 190, 193 to 202, 219 to 221, 233, and 351 to 353; or
(iii) a base sequence having 90% or more sequence identity with a base sequence selected from the group consisting of SEQ ID NOs: 167 to 171, 175 to 177, 180 to 182, 190, 193 to 202, 219 to 221, 233, and 351 to 353,
e.g., the base sequence (i).

In one embodiment, the antisense oligonucleotide of the present invention is selected from those having a ratio (average value) of the ATN-1 gene expression level obtained by administrating the antisense oligonucleotide to A204 cells to the ATN-1 gene expression level obtained by administrating OMe-6 or NRH-71, a positive control antisense oligonucleotide to A204 cells, of 0.5 or less in any of Examples, as measured by a method described in Examples of the present specification. Examples of such antisense oligonucleotides include antisense oligonucleotides consisting of 15 to 22 nucleotides complementary to a nucleic acid comprising at least 15 consecutive bases in a target region selected from the group consisting of positions 1587 to 1616, 2712 to 2735, and 4180 to 4199 of the base sequence of SEQ ID NO: 471. Other examples include antisense oligonucleotides consisting of 15 to 22 nucleotides complementary to a nucleic acid comprising at least 15 consecutive bases in a target region selected from the group consisting of positions 1587 to 1606, 1597 to 1616, 2712 to 2731, 2716 to 2735, and 4180 to 4199 of the base sequence of SEQ ID NO: 471.

An example of the antisense oligonucleotide complementary to a nucleic acid comprising at least 15 consecutive bases in the target sequence includes an antisense oligonucleotide comprising or consisting of:
(i) a base sequence selected from the group consisting of SEQ ID NOs: 170, 177, 196, 199, and 221;
(ii) a base sequence having addition, deletion, or substitution of one or several base(s) in a base sequence selected from the group consisting of SEQ ID NOs: 170, 177, 196, 199, and 221; or
(iii) a base sequence having 90% or more sequence identity with a base sequence selected from the group consisting of SEQ ID NOs: 170, 177, 196, 199, and 221,
e.g., the base sequence (i).

In one embodiment, the present invention relates to an antisense oligonucleotide consisting of 15 to 22 nucleotides complementary to a nucleic acid comprising at least 15 consecutive bases in a target region selected from the group consisting of positions 468 to 487, 474 to 493, 583 to 602, 586 to 605, 619 to 638, 770 to 789, 774 to 793, 775 to 794, 778 to 797, 804 to 823, 851 to 870, 1160 to 1179, 1162 to 1181, 1170 to 1189, 1173 to 1192, 1205 to 1224, 1210 to 1229, 1216 to 1235, 1217 to 1236, 1219 to 1238, 1385 to 1404, 1441 to 1460, 1818 to 1837, 1902 to 1921, 1905 to 1924, 1908 to 1927, 1914 to 1933, 1931 to 1950, 2117 to 2136, 2749 to 2768, 3100 to 3119, and 4125 to 4144 of a base sequence of SEQ ID NO: 471, or a pharmaceutically acceptable salt thereof, or a hydrate thereof.

An example of the antisense oligonucleotide complementary to a nucleic acid comprising at least 15 consecutive bases in the target sequence includes an antisense oligonucleotide comprising or consisting of:
(i) a base sequence selected from the group consisting of SEQ ID NOs: 7, 10, 12, 13, 25, 27, 28, 30, 61 to 65, 68 to 72, 76 to 78, 83 to 85, 90, 91, 112 to 114, 137, 146, and 159;
(ii) a base sequence having addition, deletion, or substitution of one or several base(s) in a base sequence selected from the group consisting of SEQ ID NOs: 7, 10, 12, 13, 25, 27, 28, 30, 61 to 65, 68 to 72, 76 to 78, 83 to 85, 90, 91, 112 to 114, 137, 146, and 159; or
(iii) a base sequence having 90% or more sequence identity with a base sequence selected from the group consisting of SEQ ID NOs: 7, 10, 12, 13, 25, 27, 28, 30, 61 to 65, 68 to 72, 76 to 78, 83 to 85, 90, 91, 112 to 114, 137, 146, and 159,
e.g., the base sequence (i).

In one embodiment, the antisense oligonucleotide of the present invention inhibits the function of a target region. As used herein, the phrase “inhibit the function of a target region” encompasses one or more of cleaving with RNaseH of a genome RNA comprising the target region in which a double strand is formed by binding of the antisense oligonucleotide to the target region, inhibiting replication of the genome RNA comprising the target region, inhibiting translation when the target region is to be translated, and inhibiting transcription of the genome RNA comprising the target region. As used herein, the term “sub-genome RNA” refers to an RNA that is shorter than a genome RNA synthesized with the RNA-dependent RNA polymerase using a part of a negative-strand RNA as a template, which is synthesized with an RNA-dependent RNA polymerase using a positive-strand genome RNA as a template, and means an RNA working as an mRNA for viral protein synthesis (translation).

The antisense oligonucleotide of the present invention may be easily synthesized using various automated synthesizers (e.g., AKTA oligopilot plus 10/100 (GE Healthcare)). Alternatively, the synthesis may also be entrusted to a third-party organization (e.g., Promega Corp. or Takara Co.).

In one embodiment, the present invention relates to a pharmaceutical composition comprising an antisense oligonucleotide, or a pharmaceutically acceptable salt thereof, or a hydrate thereof. When the antisense nucleotide of the present invention is to be administered to a subject, the pharmaceutical composition of the present invention may comprise a carrier to promote delivery of the antisense nucleotide. Such a carrier is not particularly limited as far as it is pharmaceutically acceptable, and examples thereof include cationic carriers such as cationic liposomes, or cationic polymers, or carriers using viral envelope. The cationic liposomes include, for example, liposomes composed of 2-O-(2-diethylaminoethyl)carbamoyl-1,3-O-dioleoylglycerol and phospholipids as the essential constituents (hereinafter referred to as “liposome A”), Oligofectamine (registered trademark) (manufactured by Invitrogen Corp.), Lipofectin (registered trademark) (manufactured by Invitrogen Corp.), Lipofectamine (registered trademark) (manufactured by Invitrogen Corp.), Lipofectamine 2000 (registered trademark) (manufactured by Invitrogen Corp.), DMRIE-C (registered trademark) (manufactured by Invitrogen Corp.), GeneSilencer (registered trademark) (manufactured by Gene Therapy Systems), TransMessenger (registered trademark) (manufactured by QIAGEN, Inc.), TransIT TKO (registered trademark) (manufactured by Mirus), and Nucleofector II (Lonza). Examples of the cationic polymers include, for example, JetSI (registered trademark) (manufactured by Qbiogene, Inc.), and Jet-PEI (registered trademark) (polyethylenimine, manufactured by Qbiogene, Inc.). An example of carriers using viral envelop includes GenomeOne (registered trademark) (HVJ-E liposome, manufactured by Ishihara Sangyo). Alternatively, the medical devices described in Japanese Patent No. 2924179, and the cationic carriers described in Japanese Domestic Re-Publication of PCT Application Nos. 2006/129594 and 2008/096690 may be used as well.

In one embodiment, the antisense oligonucleotide of the present invention may be in the form of a complex (conjugate) with a lipid or the like in the pharmaceutical composition to promote delivery of the antisense oligonucleotide. For example, as described in Bijsterbosch, M. K. et al., (2000) Nucleic Acid Res., 28, 2717-2725, the antisense oligonucleotide may be in the form of a conjugate with cholesterol.

The pharmaceutical composition of the present invention may comprise pharmaceutically acceptable additives in addition to the antisense oligonucleotide, or a pharmaceutically acceptable salt thereof, or a hydrate thereof and optionally the carrier described above. Examples of such additives are emulsification aids (e.g., fatty acids having 6 to 22 carbon atoms and their pharmaceutically acceptable salts, albumin and dextran), stabilizers (e.g., cholesterol, phosphatidic acid, mannitol, and sorbitol), isotonizing agents (e.g., sodium chloride, glucose, maltose, lactose, sucrose, and trehalose), and pH adjusting agents (e.g., hydrochloric acid, sulfuric acid, phosphoric acid, acetic acid, sodium hydroxide, potassium hydroxide, and triethanolamine). One or more of these additives can be used. The content of the additive in the composition of the present invention is appropriately 90 wt % or less, preferably 70 wt % or less, and more preferably 50 wt % or less.

The preparation method of the pharmaceutical composition of the present invention is not limited, and the preparation may be conducted by, for example, adding the antisense oligonucleotide of the present invention to a dispersion of the carrier, and appropriately stirring the resultant. The additive may be added in an appropriate step either before or after the addition of the antisense oligonucleotide of the present invention. An aqueous solvent used in adding the antisense oligonucleotide of the present invention is not particularly limited as long as it is pharmaceutically acceptable, and examples include injectable water, injectable distilled water, an electrolyte fluid such as physiological saline, and a sugar solution such as a glucose solution, or a maltose solution. Those skilled in the art can appropriately choose conditions for pH and temperature to be employed in this case.

The pharmaceutical composition of the present invention may be prepared into, for example, a liquid form or its lyophilized preparation. The lyophilized preparation can be prepared by lyophilizing the composition of the present invention in a liquid form in a conventional manner. The lyophilization can be performed, for example, by appropriately sterilizing the composition of the present invention in a liquid form, dispensing an aliquot into a vial container, performing preliminary freezing for 2 hours at conditions in a range of about −40° C. to −20° C., performing a primary drying in a range of about 0° C. to 10° C. under reduced pressure, and then performing a secondary drying in a range of about 15° C. to 25° C. under reduced pressure. In general, the lyophilized preparation of the composition of the present invention can be obtained by replacing the content of the vial with nitrogen gas and capping the resultant.

The lyophilized preparation of the pharmaceutical composition of the present invention can be used in general upon reconstitution by adding an optional suitable solution (reconstitution liquid). Examples of such a reconstitution liquid include injectable water, physiological saline and other general infusion fluids. A volume of the reconstitution liquid may vary depending on the intended use, etc., is not particularly limited, and is suitably 0.5-fold to 2-fold greater than the volume prior to the lyophilization or no more than 500 mL.

It is desired to control a dose of the pharmaceutical composition of the present invention to be administered by taking the following factors into account: the type and dosage form of the antisense oligonucleotide of the present invention contained; patients' conditions including age, body weight, etc.; administration route; and the characteristics and extent of the disease. A single dose for an adult calculated as the amount of the antisense oligonucleotide of the present invention can be 0.01 mg to 20 mg per kg body weight, preferably 0.03 mg to 10 mg per kg body weight, more preferably 0.05 mg to 4 mg per kg body weight, and further preferably 0.1 mg to 2 mg per kg body weight. The frequency of administration may be once per 1 to 3 days, once per week, or once per 2 to 3 weeks. This numerical range may vary occasionally depending on the type of the target disease, the administration form and the target molecule. Therefore, a dose or frequency of administration lower than these ranges may be sufficient in some occasion and conversely, a dose or frequency of administration higher than these ranges may be required occasionally.

The administration form of the pharmaceutical composition of the present invention is not particularly limited as long as it is pharmaceutically acceptable form of administration, and can be chosen depending upon method of treatment. Examples include intravenous administration, intraarterial administration, intramuscular administration, subcutaneous administration, oral administration, tissue administration, transdermal administration, pulmonary administration, nasal administration, and administration to central nerve. Examples of the administration to central nerve include intrathecal administration, intracranial administration, e.g., intracerebroventricular administration or lateral ventricle administration, intraparenchymal administration, and administration to leptomeninges (pia mater). Also, dosage forms which are available for the composition of the present invention are not particularly limited, and include, for example, various injections, oral agents, drips, inhalations, ointments, lotions, and poultices.

Examples of the subject to which the antisense oligonucleotide or the pharmaceutical composition of the present invention is administered include mammals, including primates such as a human, experimental animals such as a rat, a mouse, and a brown rat, and domestic animals such as a pig, a cow, a horse, and sheep, and the subject is preferably a human.

In one embodiment, the present invention relates to a method for treating and/or preventing dentatorubral-pallidoluysian atrophy (DRPLA), comprising administering, to a subject, the antisense oligonucleotide, or a pharmaceutically acceptable salt thereof, or a hydrate thereof, or the pharmaceutical composition of the present invention. The pharmaceutical composition, and the dose, the administration route and the like thereof in the present embodiment are the same as those described herein.

As used herein, treatment of dentatorubral-pallidoluysian atrophy (DRPLA) encompasses one or more of relief, improvement, and remission of dentatorubral-pallidoluysian atrophy or symptoms thereof (e.g., one or more of ataxia, myoclonus, epilepsy, and progressive intellectual deterioration in a child patient, and ataxia, choreoathetosis, dementia, and personality change in an adult patient). As used herein, prevention of DRPLA encompasses reduction of risks of causing DRPLA or the symptoms thereof.

### EXAMPLES

Hereinafter, the present invention will be described in more detail with reference to Examples and Test Examples below, but the present invention is not limited thereto.

The term “gapmer” is defined as an oligomer compound, an oligonucleotide in general, having a center region of 2′-deoxyoligonucleotide flanked by two adjacent segments of non-deoxyoligonucleotides. The center region is called “gap”, and the adjacent segments are called “wings”. The gapmers used in the present examples have a gap of 10 nucleotides flanked by two adjacent wings of 5 nucleotides. These are called 5-10-5 gapmers.

### Example 1: Synthesis of Gapmer (Oligonucleotide)

A gapmer (oligonucleotide) used in this example can be produced generally in accordance with a method described in W. Brad Wan et al., Nucleic Acid Research, Vol. 42, No. 22 13456 (2014), etc.

Among the gapmers used in the present example, some specific oligonucleotides having a 2′-methoxy (2′-OMe) group represented by the following formula (a) in a nucleoside of the wing were synthesized referring to the method described in W. Brad Wan et al., Nucleic Acid Research, Vol. 42, No. 22 13456 (2014) as described above. The others were obtained by entrusting the synthesis to GeneDesign Inc.

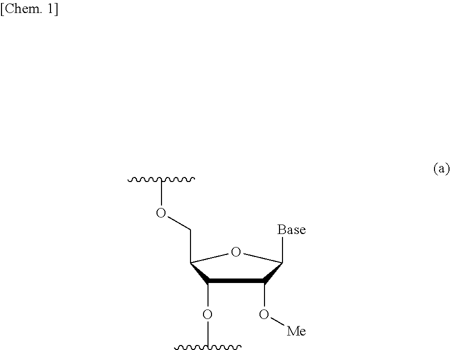

wherein Base represents cytosine (C), uracil (U), adenine (A), or guanine (G), and Me represents methyl.

Among the gapmers used in the present example, some specific oligonucleotides having a 2′-methoxyethyl (2′-O-MOE) group represented by the following formula (b) in a nucleoside of the wing were synthesized referring to the method described in W. Brad Wan et al., Nucleic Acid Research, Vol. 42, No. 22 13456 (2014) as described above.

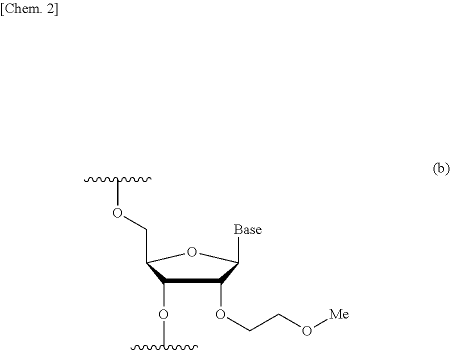

wherein Base represents 5-methylcytosine (C), thymine (T), adenine (A), or guanine (G), and Me represents methyl.

A 20 mer gapmer (oligonucleotide) to be tested having the 2′-OMe group represented by the formula (a) or the 2′-O-MOE group represented by the formula (b) in a nucleoside of the wing was synthesized at 1 μmol scale with an automated nucleic acid synthesizer, NTS M-8-MX DNA/RNA (NIHON TECHNO SERVICE CO., LTD.). The extension of chain length was performed in accordance with standard phosphoramidite protocol (solid-phase carrier: Glen UnySupport, DDTT ([(N,N-Dimethylaminomethylidene)amino]-3H-1,2,4-dithiazoline-3-thione), etc. was used for sulfuration, 0.02 M iodine/tetrahydrofuran:pyridine:water=7:1:2, etc. was used for oxidation). Thus, an oligonucleotide in which a hydroxyl group at the 5′-position at the end was protected with a dimethoxytrityl (DMTr) group, and the 3′-position was carried on a solid-phase was obtained. Subsequently, after removing the DMTr group by treatment with deblocking solution-1 obtained from FUJIFILM Wako Pure Chemical Corporation, the resultant was subjected to ammonia treatment, and thus, an objective product was cut out from the solid-phase carrier. The solvent was distilled off, and thus the obtained crude product was purified by reverse phase HPLC to give an objective product.

### Example 2: Design of Gapmer (Oligonucleotide) to be Tested

A gapmer (oligonucleotide) to be tested was designed as an antisense oligonucleotide (ASO) targeting human atrophin-1 (ATN1) (Gen Bank: NM_001007026, SEQ ID NO: 471). Sequences of oligonucleotides expressed by DNA bases are shown in Tables 1, 2, 3, and 4.

In Tables 1 and 2, all oligonucleotides are 5-10-5 gapmers, and bonds between nucleosides are all phosphorothioates (P═S). In gapmers having a 2′-OMe group of Table 1, T (thymine) in the wing region is actually U (uracil). In gapmers having a 2′-O-MOE group of Table 2, C (cytosine) in the gap region and the wing regions is actually methyl C, and T in the wing regions is actually T.

TABLE 1
Table 1 Gapmer (oligonucleotide) to be
tested having 2′-OMe group in wing nucleoside

| | | Start | End | | | |
|---|---|---|---|---|---|---|
| | | position | position | | | |
| | | in | in | | | SEQ |
| ASO base sequence | ASO | SEQ ID | SEQ ID | Target | ASO | ID |
| (DNA) 5′ to 3′ | No. | NO: 471 | NO: 471 | exon | name | NO: |
| TCCTCCCACTTGGCGTCCAG | 1 | 14 | 33 | 1 | OMe-24 | 1 |
| AGAAGCAGAGCGGGAGGCCC | 2 | 54 | 73 | 1 | OMe-25 | 2 |
| TTCCCCACAGCTGTGGTCTG | 3 | 148 | 167 | 2 | OMe-97 | 3 |
| GTGGAGGCTTCCTCTACTCG | 4 | 413 | 432 | 4 | OMe-112 | 4 |
| AGGAGGAAAGAGTGGAGGTG | 5 | 723 | 742 | 5 | OMe-37 | 5 |
| GGTGCTGTCTGGCGGTTGAG | 6 | 747 | 766 | 5 | OMe-98 | 6 |
| TTCAAAGCTAGCCTCTGGCT | 7 | 774 | 793 | 5 | OMe-38 | 7 |
| TTAGGGCCCCCCACTGATGA | 8 | 965 | 984 | 5 | OMe-129 | 8 |
| GGCAGGTTCGGTGTCACATG | 9 | 1127 | 1146 | 5 | OMe-43 | 9 |
| AGAGGCTGATGCATTGTTGA | 10 | 1170 | 1189 | 5 | OMe-44 | 10 |
| TGGTTGGGCCCCCAGGCCAG | 11 | 1194 | 1213 | 5 | OMe-105 | 11 |
| GCAGATGACCAGGTAGTGGT | 12 | 1210 | 1229 | 5 | OMe-45 | 12 |
| GGAGAGGGCAGATGACCAGG | 13 | 1217 | 1236 | 5 | OMe-106 | 13 |
| GGTGAAGGAGCCAGAGTTGG | 14 | 1289 | 1308 | 5 | OMe-47 | 14 |
| GAGACAGAGAGGCTTGTTGG | 15 | 1484 | 1503 | 5 | OMe-23 | 15 |
| ATTGGAGACAGAGAGGCTTG | 16 | 1488 | 1507 | 5 | OMe-22 | 16 |
| CTGATTGGAGACAGAGAGGC | 17 | 1491 | 1510 | 5 | OMe-14 | 17 |
| GGCTGATTGGAGACAGAGAG | 18 | 1493 | 1512 | 5 | OMe-21 | 18 |
| AGAGAAGGCTGAGTATACTT | 19 | 1517 | 1536 | 5 | OMe-5 | 19 |
| ACCCTGGCTCCACACAGCCT | 20 | 1545 | 1564 | 5 | OMe-15 | 20 |
| GCATTGCTGTTGGCTAAGAG | 21 | 1592 | 1611 | 5 | OMe-50 | 21 |
| CTGTTGCTGTTGCTGGTGGT | 22 | 1686 | 1705 | 5 | OMe-131 | 22 |
| GGCCCAGAGTTTCCGTGATG | 23 | 1748 | 1767 | 5 | OMe-52 | 23 |
| GGAGCTACCGCCCTCCAGTG | 24 | 1794 | 1813 | 5 | OMe-53 | 24 |
| CATGGCGTAAGGGTGTGCGT | 25 | 1818 | 1837 | 5 | OMe-107 | 25 |
| AGGACACCTGGCTGTGAGGT | 26 | 1894 | 1913 | 5 | OMe-110 | 26 |
| GCCTGCTTGGCTGTAGGACA | 27 | 1908 | 1927 | 5 | OMe-55 | 27 |
| GAAGAGACTGGAGGGCCATT | 28 | 1931 | 1950 | 5 | OMe-111 | 28 |
| CCACGGTGGCAATGACCGTG | 29 | 2086 | 2105 | 5 | OMe-116 | 29 |
| GAGGCCGTTTTGTAGCCTGC | 30 | 2117 | 2136 | 5 | OMe-117 | 30 |
| GGCTGTCTTTCCGTACGGTG | 31 | 2151 | 2170 | 5 | OMe-60 | 31 |
| GCGACCCGGGTTTGTATCCG | 32 | 2206 | 2225 | 5 | OMe-118 | 32 |
| CGAGGTTCCTCGATAGCCCG | 33 | 2253 | 2272 | 5 | OMe-62 | 33 |
| TCCCACGGTGGGCGAGCCCG | 34 | 2301 | 2320 | 5 | OMe-63 | 34 |
| GGTGGTGGCAGCGATGGCAG | 35 | 2354 | 2373 | 5 | OMe-64 | 35 |
| CAGCTTGGAGCCCTCCAGTG | 36 | 2598 | 2617 | 6 | OMe-68 | 36 |
| CTGCTCGGCCTCGCGCCGCA | 37 | 2649 | 2668 | 6 | OMe-69 | 37 |
| TCTTCGCGCGCGCGCTGCTC | 38 | 2663 | 2682 | 6 | OMe-96 | 38 |
| TCTTTCTCGCGTTCCCGCTC | 39 | 2699 | 2718 | 6 | OMe-70 | 39 |
| TCAAGCTCGCGCTCCTTCTC | 40 | 2729 | 2748 | 6 | OMe-132 | 40 |
| ACGCTGCGTTCAAGCTCGCG | 41 | 2738 | 2757 | 6 | OMe-71 | 41 |
| GCATTCCACCGGAGCACGGC | 42 | 2775 | 2794 | 7 | OMe-133 | 42 |
| CGCACTGCCCGGTTCAAATG | 43 | 2826 | 2845 | 7 | OMe-73 | 43 |
| GCTTCCCGTTCCCTCTCCCG | 44 | 3032 | 3051 | 7 | OMe-77 | 44 |
| CACGGAGGTCTCGTTCACGG | 45 | 3052 | 3071 | 7 | OMe-135 | 45 |
| GAAAGGGATCCAAGCCCGGC | 46 | 3136 | 3155 | 7 | OMe-79 | 46 |
| GCCAGGCTGCAGAGCCAGGC | 47 | 3168 | 3187 | 7 | OMe-80 | 47 |
| CTAGACGTTCTCGCTCCAGG | 48 | 3232 | 3251 | 7 | OMe-136 | 48 |
| GCTGGCCCAGCTGCCAGCGC | 49 | 3251 | 3270 | 7 | OMe-82 | 49 |
| GTGCAGGTGCGAGTGGATGT | 50 | 3408 | 3427 | 7 | OMe-83 | 50 |
| AGAACTTCGTTCTCGTGCAG | 51 | 3563 | 3582 | 8 | OMe-86 | 51 |
| GAGGCCGGCAGGTCCCGGTA | 52 | 3608 | 3627 | 9 | OMe-87 | 52 |
| GCAGAAAGGGAGGCCGGCAG | 53 | 3617 | 3636 | 9 | OMe-123 | 53 |
| GTGCATGGCCTGCAGCTGAT | 54 | 3654 | 3673 | 9 | OMe-88 | 54 |
| CATGCAGCCACTGCTGCTGT | 55 | 3709 | 3728 | 9 | OMe-89 | 55 |
| GCAGCGGCACACTGTGCAGC | 56 | 3739 | 3758 | 9 | OMe-90 | 56 |
| CCCTCCCTCCCCCACTCTCT | 57 | 3949 | 3968 | 10 | OMe-9 | 57 |
| GCCACCTCCCCACCTCTGCA | 58 | 3999 | 4018 | 10 | OMe-10 | 58 |

TABLE 2
Table 2 Gapmer (oligonucleotide) to be
tested having 2′-O-MOE group in wing nucleoside

| | | Start | End | | | |
|---|---|---|---|---|---|---|
| | | position | position | | | |
| | | in | in | | | SEQ |
| ASO base sequence | ASO | SEQ ID | SEQ ID | Target | ASO | ID |
| (DNA) 5′ to 3′ | No. | NO: 471 | NO: 471 | exon | name | NO: |
| GCAGGGGACAGCCTTGAAGT | 59 | 34 | 53 | 1 | DRA-3 | 59 |
| CTCGGGCCTTCTTGGCTGTC | 60 | 397 | 416 | 3 | DRA-29 | 60 |
| CTCACTTTCACTCTCTGAGA | 61 | 468 | 487 | 4 | DRA-9 | 61 |
| GGTCTCCTCACTTTCACTCT | 62 | 474 | 493 | 4 | DRA-96 | 62 |
| GGTCGCTGCTGCCATCATCA | 63 | 583 | 602 | 5 | DRA-105 | 63 |
| TAGGGTCGCTGCTGCCATCA | 64 | 586 | 605 | 5 | DRA-13 | 64 |
| GGGACGTGCTTCGGTTGTCC | 65 | 619 | 638 | 5 | DRA-14 | 65 |
| CTGTCGAGGGGTGCTGTCTG | 66 | 756 | 775 | 5 | DRA-108 | 66 |
| TGGCTGTCGAGGGGTGCTGT | 67 | 759 | 778 | 5 | DRA-109 | 67 |
| AAGCTAGCCTCTGGCTGTCG | 68 | 770 | 789 | 5 | DRA-112 | 68 |
| GTTCAAAGCTAGCCTCTGGC | 69 | 775 | 794 | 5 | NRH-156 | 69 |
| GGGGTTCAAAGCTAGCCTCT | 70 | 778 | 797 | 5 | DRA-113 | 70 |
| ATATCCAGTGGGTGTCACAG | 71 | 804 | 823 | 5 | DRA-16 | 71 |
| GGAGGAGCCTGGAACATTCG | 72 | 851 | 870 | 5 | DRA-17 | 72 |
| GGTCTCAGGGCAGGTGGGGG | 73 | 1148 | 1167 | 5 | DRA-128 | 73 |
| AGGGGTCTCAGGGCAGGTGG | 74 | 1151 | 1170 | 5 | DRA-129 | 74 |
| TTGAGGGGTCTCAGGGCAGG | 75 | 1154 | 1173 | 5 | DRA-130 | 75 |
| GCATTGTTGAGGGGTCTCAG | 76 | 1160 | 1179 | 5 | NRH-177 | 76 |
| ATGCATTGTTGAGGGGTCTC | 77 | 1162 | 1181 | 5 | NRH-171 | 77 |
| AGAGGCTGATGCATTGTTGA | 78 | 1170 | 1189 | 5 | NRH-178 | 10 |
| GGGAGAGGCTGATGCATTGT | 79 | 1173 | 1192 | 5 | NRH-174 | 78 |
| CAGGCCAGGGGGAGAGGCTG | 80 | 1182 | 1201 | 5 | NRH-175 | 79 |
| CCCCAGGCCAGGGGGAGAGG | 81 | 1185 | 1204 | 5 | NRH-176 | 80 |
| GGCCCCCAGGCCAGGGGGAG | 82 | 1188 | 1207 | 5 | NRH-181 | 81 |
| TTGGGCCCCCAGGCCAGGGG | 83 | 1191 | 1210 | 5 | NRH-182 | 82 |
| TGGTTGGGCCCCCAGGCCAG | 84 | 1194 | 1213 | 5 | NRH-179 | 11 |
| TGACCAGGTAGTGGTTGGGC | 85 | 1205 | 1224 | 5 | NRH-168 | 83 |
| GCAGATGACCAGGTAGTGGT | 86 | 1210 | 1229 | 5 | NRH-180 | 12 |
| GAGAGGGCAGATGACCAGGT | 88 | 1216 | 1235 | 5 | NRH-162 | 84 |
| GGAGAGGGCAGATGACCAGG | 89 | 1217 | 1236 | 5 | NRH-150 | 13 |
| GGGGAGAGGGCAGATGACCA | 90 | 1219 | 1238 | 5 | NRH-158 | 85 |
| CCATGGCGTGGGGAGAGGGC | 91 | 1228 | 1247 | 5 | DRA-133 | 85 |
| GTCCCATGGCGTGGGGAGAG | 92 | 1231 | 1250 | 5 | DRA-134 | 87 |
| CCCTGTCCCATGGCGTGGGG | 93 | 1235 | 1254 | 5 | DRA-21 | 88 |
| GGCCCTTCTCTGGGCCAGGA | 94 | 1270 | 1289 | 5 | DRA-31 | 89 |
| GAAGAGGAGGCTGCTGCAGA | 95 | 1385 | 1404 | 5 | DRA-24 | 90 |
| TGGGCAATGCCTGGGAAGCT | 96 | 1441 | 1460 | 5 | DRA-25 | 91 |
| AGTATACTTGGGGGGCTGAT | 97 | 1506 | 1525 | 5 | DRA-26 | 92 |
| CAGCCTGGGATGGGAGAGAA | 98 | 1531 | 1550 | 5 | DRA-32 | 93 |
| TAAGAGGCGGCCATAGGGAG | 99 | 1578 | 1597 | 5 | DRA-33 | 94 |
| CTGTTGGCTAAGAGGCGGCC | 100 | 1586 | 1605 | 5 | DRA-115 | 95 |
| TTGCTGTTGGCTAAGAGGCG | 101 | 1589 | 1608 | 5 | DRA-116 | 96 |
| TGGGCATTGCTGTTGGCTAA | 102 | 1595 | 1614 | 5 | DRA-117 | 97 |
| GGATGGGCATTGCTGTTGGC | 103 | 1598 | 1617 | 5 | DRA-118 | 98 |
| CCTGGATGGGCATTGCTGTT | 104 | 1601 | 1620 | 5 | DRA-119 | 99 |
| GGGCCTGGATGGGCATTGCT | 105 | 1604 | 1623 | 5 | DRA-34 | 100 |
| AAGGGGCCTGGATGGGCATT | 106 | 1607 | 1626 | 5 | DRA-120 | 101 |
| GGGAAGGGGCCTGGATGGGC | 107 | 1610 | 1629 | 5 | DRA-121 | 102 |
| GAAATGCTCCAGGAGGAGGG | 108 | 1768 | 1787 | 5 | DRA-36 | 103 |
| GCAGGTGTGCTGGCCCTGGT | 109 | 1870 | 1889 | 5 | NRH-154 | 104 |
| GGCAGGTGTGCTGGCCCTGG | 110 | 1871 | 1890 | 5 | NRH-160 | 105 |
| GGGGGCAGGTGTGCTGGCCC | 111 | 1874 | 1893 | 5 | DRA-140 | 106 |
| GGTGGGGGCAGGTGTGCTGG | 112 | 1877 | 1896 | 5 | DRA-141 | 107 |
| TGAGGTGGGGGCAGGTGTGC | 113 | 1880 | 1899 | 5 | DRA-142 | 108 |
| CTGTGAGGTGGGGGCAGGTG | 114 | 1883 | 1902 | 5 | DRA-143 | 109 |
| TGGCTGTGAGGTGGGGGCAG | 115 | 1886 | 1905 | 5 | DRA-144 | 110 |
| ACCTGGCTGTGAGGTGGGGG | 116 | 1889 | 1908 | 5 | DRA-145 | 111 |
| TTGGCTGTAGGACACCTGGC | 117 | 1902 | 1921 | 5 | DRA-148 | 112 |
| TGCTTGGCTGTAGGACACCT | 118 | 1905 | 1924 | 5 | DRA-149 | 113 |
| ATTGGGGCCTGCTTGGCTGT | 119 | 1914 | 1933 | 5 | DRA-152 | 114 |
| GGCGCCCCTTGAGGGCCCTG | 120 | 2015 | 2034 | 5 | DRA-42 | 115 |
| TGGCGAGGAAGCCACGGTGG | 121 | 2097 | 2116 | 5 | DRA-43 | 116 |
| GCTGGCGAGGAAGCCACGGT | 122 | 2099 | 2118 | 5 | DRA-185 | 117 |
| GAGGCCGTTTTGTAGCCTGC | 124 | 2117 | 2136 | 5 | DRA-191 | 30 |
| GGCCCAGGTGGGGAGGCCGT | 125 | 2129 | 2148 | 5 | DRA-195 | 118 |
| GCCCCCGGGGACGGGGCTCT | 126 | 2165 | 2184 | 5 | DRA-44 | 119 |
| AGCCCGGTGGGGTCCCCGTT | 127 | 2239 | 2258 | 5 | DRA-46 | 120 |
| AGCCCGGCTTGAAGGTCCCT | 128 | 2287 | 2306 | 5 | DRA-47 | 121 |
| GTGGCAGGGGCCCAGGTCCC | 129 | 2317 | 2336 | 5 | DRA-48 | 122 |
| CAGGCCTGAGGGCCCCGCAG | 130 | 2337 | 2356 | 5 | DRA-49 | 123 |
| CTCAGGGGCGGCCCTGAGGC | 131 | 2387 | 2406 | 5 | DRA-53 | 124 |
| GGCCCGCTTCTTGGCCAGCT | 132 | 2613 | 2632 | 6 | DRA-56 | 125 |
| CGCACCTTCTCCACCAGGTC | 133 | 2633 | 2652 | 6 | DRA-57 | 126 |
| CGCTCCTTTTCTTCGCGCGC | 134 | 2672 | 2691 | 6 | DRA-58 | 127 |
| CGCTCTTTCTCGCGTTCCCG | 135 | 2702 | 2721 | 6 | DRA-156 | 128 |
| TCGCGCTCTTTCTCGCGTTC | 136 | 2705 | 2724 | 6 | DRA-157 | 129 |
| CGCTCGCGCTCTTTCTCGCG | 137 | 2708 | 2727 | 6 | DRA-158 | 130 |
| TCGCGCTCGCGCTCTTTCTC | 138 | 2711 | 2730 | 6 | DRA-159 | 131 |
| TTCTCGCGCTCGCGCTCTTT | 139 | 2714 | 2733 | 6 | DRA-59 | 132 |
| TCCTTCTCGCGCTCGCGCTC | 140 | 2717 | 2736 | 6 | DRA-160 | 133 |
| CGCTCCTTCTCGCGCTCGCG | 141 | 2720 | 2739 | 6 | DRA-161 | 134 |
| TCGCGCTCCTTCTCGCGCTC | 142 | 2723 | 2742 | 6 | DRA-162 | 135 |
| AGCTCGCGCTCCTTCTCGCG | 143 | 2726 | 2745 | 6 | DRA-163 | 136 |
| GAGCCAACTTCACGCTGCGT | 144 | 2749 | 2768 | 6 | DRA-60 | 137 |
| TGGGCCCAGAGATGGGCATT | 145 | 2790 | 2809 | 7 | DRA-61 | 138 |
| CACCGCACTGCCCGGTTCAA | 146 | 2829 | 2848 | 7 | DRA-122 | 139 |
| AGCCACCGCACTGCCCGGTT | 147 | 2832 | 2851 | 7 | DRA-123 | 140 |
| TGTAGCCACCGCACTGCCCG | 148 | 2835 | 2854 | 7 | DRA-124 | 141 |
| CACTGTAGCCACCGCACTGC | 149 | 2838 | 2857 | 7 | DRA-125 | 142 |
| GGGCACTGTAGCCACCGCAC | 150 | 2841 | 2860 | 7 | DRA-62 | 143 |
| GTAGGGGGGCACTGTAGCCA | 151 | 2847 | 2866 | 7 | DRA-126 | 144 |
| GGTCCACTGCCCCCAGGGGC | 152 | 2959 | 2978 | 7 | DRA-64 | 145 |
| GGGGTTCCAGCTCACTAGGC | 153 | 3100 | 3119 | 7 | DRA-66 | 146 |
| AAAGGGTGCAGGCCAGGTGG | 154 | 3188 | 3207 | 7 | DRA-68 | 147 |
| GCCTCTCAGCTGCCAGCCGC | 155 | 3298 | 3317 | 7 | DRA-69 | 148 |
| CCAGGGCCGCCACCCTTTCT | 156 | 3325 | 3344 | 7 | DRA-70 | 149 |
| TAAGGGGCAGCAAAGAGCTG | 157 | 3590 | 3609 | 8 | DRA-74 | 150 |
| TGAGCTGCTGACATCGGGGC | 158 | 3635 | 3654 | 9 | DRA-75 | 151 |
| AGCTCAGCTGACTGTGCGTG | 159 | 3671 | 3690 | 9 | DRA-76 | 152 |
| GTTCCAGCGCCAAGCGCTGC | 160 | 3691 | 3710 | 9 | DRA-77 | 153 |
| CTCCTGGGCAGGCAGCGGCA | 161 | 3750 | 3769 | 9 | DRA-78 | 154 |
| CAGGCCAAGGGCACGGTGGG | 162 | 3877 | 3896 | 10 | DRA-81 | 155 |
| TGGCCTTCTGTCTGTCCCTC | 163 | 3964 | 3983 | 10 | DRA-84 | 156 |
| CTGCACACCACATCGGGCCT | 164 | 3984 | 4003 | 10 | DRA-85 | 157 |
| GTCCCCATCCTCGCCACCTC | 165 | 4011 | 4030 | 10 | DRA-86 | 158 |
| AATCACACCAACGGGGCAGG | 166 | 4125 | 4144 | 10 | DRA-87 | 159 |
| TGGCACACGATGCTACGCAA | 167 | 4170 | 4189 | 10 | DRA-172 | 160 |
| GGGTGGCACACGATGCTACG | 168 | 4173 | 4192 | 10 | DRA-89 | 161 |
| CAGGGGTGGCACACGATGCT | 169 | 4176 | 4195 | 10 | DRA-173 | 162 |
| GGGCAGGGGTGGCACACGAT | 170 | 4179 | 4198 | 10 | DRA-174 | 163 |
| CTACTCGGGCCTTCTTGGCT | 171 | 400 | 419 | 3 | | 164 |
| CCTCTACTCGGGCCTTCTTG | 172 | 403 | 422 | 3 | | 165 |
| CTTCCTCTACTCGGGCCTTC | 173 | 406 | 425 | 3 | | 166 |
| GGCTAAGAGGCGGCCATAGG | 174 | 1581 | 1600 | 5 | | 167 |
| GTTGGCTAAGAGGCGGCCAT | 175 | 1584 | 1603 | 5 | | 168 |
| TGTTGGCTAAGAGGCGGCCA | 176 | 1585 | 1604 | 5 | | 169 |
| GCTGTTGGCTAAGAGGCGGC | 177 | 1587 | 1606 | 5 | | 170 |
| TGCTGTTGGCTAAGAGGCGG | 178 | 1588 | 1607 | 5 | | 171 |
| ATTGCTGTTGGCTAAGAGGC | 179 | 1590 | 1609 | 5 | | 172 |
| CATTGCTGTTGGCTAAGAGG | 180 | 1591 | 1610 | 5 | | 173 |
| GCATTGCTGTTGGCTAAGAG | 181 | 1592 | 1611 | 5 | | 21 |
| GGCATTGCTGTTGGCTAAGA | 182 | 1593 | 1612 | 5 | | 174 |
| GGGCATTGCTGTTGGCTAAG | 183 | 1594 | 1613 | 5 | | 175 |
| ATGGGCATTGCTGTTGGCTA | 184 | 1596 | 1615 | 5 | | 176 |
| GATGGGCATTGCTGTTGGCT | 185 | 1597 | 1616 | 5 | | 177 |
| TGGATGGGCATTGCTGTTGG | 186 | 1599 | 1618 | 5 | | 178 |
| CTGGATGGGCATTGCTGTTG | 187 | 1600 | 1619 | 5 | | 179 |
| GCCTGGATGGGCATTGCTGT | 188 | 1602 | 1621 | 5 | | 180 |
| GGCCTGGATGGGCATTGCTG | 189 | 1603 | 1622 | 5 | | 181 |
| GGGGCCTGGATGGGCATTGC | 190 | 1605 | 1624 | 5 | | 182 |
| AGGTGGGGGCAGGTGTGCTG | 191 | 1878 | 1897 | 5 | | 183 |
| GAGGTGGGGGCAGGTGTGCT | 192 | 1879 | 1898 | 5 | | 184 |
| CGAGGAAGCCACGGTGGCAA | 193 | 2094 | 2113 | 5 | | 185 |
| CCTTCTCCACCAGGTCGGCC | 194 | 2629 | 2648 | 6 | | 186 |
| GCGCCGCACCTTCTCCACCA | 195 | 2637 | 2656 | 6 | | 187 |
| CTCTTTCTCGCGTTCCCGCT | 196 | 2700 | 2719 | 6 | | 188 |
| GCTCTTTCTCGCGTTCCCGC | 197 | 2701 | 2720 | 6 | | 189 |
| GCGCTCTTTCTCGCGTTCCC | 198 | 2703 | 2722 | 6 | | 190 |
| CGCGCTCTTTCTCGCGTTCC | 199 | 2704 | 2723 | 6 | | 191 |
| CTCGCGCTCTTTCTCGCGTT | 200 | 2706 | 2725 | 6 | | 192 |
| GCTCGCGCTCTTTCTCGCGT | 201 | 2707 | 2726 | 6 | | 193 |
| GCGCTCGCGCTCTTTCTCGC | 202 | 2709 | 2728 | 6 | | 194 |
| CGCGCTCGCGCTCTTTCTCG | 203 | 2710 | 2729 | 6 | | 195 |
| CTCGCGCTCGCGCTCTTTCT | 204 | 2712 | 2731 | 6 | | 196 |
| TCTCGCGCTCGCGCTCTTTC | 205 | 2713 | 2732 | 6 | | 197 |
| CTTCTCGCGCTCGCGCTCTT | 206 | 2715 | 2734 | 6 | | 198 |
| CCTTCTCGCGCTCGCGCTCT | 207 | 2716 | 2735 | 6 | | 199 |
| CTCCTTCTCGCGCTCGCGCT | 208 | 2718 | 2737 | 6 | | 200 |
| GCTCCTTCTCGCGCTCGCGC | 209 | 2719 | 2738 | 6 | | 201 |
| GCGCTCCTTCTCGCGCTCGC | 210 | 2721 | 2740 | 6 | | 202 |
| CGCGCTCCTTCTCGCGCTCG | 211 | 2722 | 2741 | 6 | | 203 |
| GCACTGTAGCCACCGCACTG | 212 | 2839 | 2858 | 7 | | 204 |
| GGCACTGTAGCCACCGCACT | 213 | 2840 | 2859 | 7 | | 205 |
| GGGGCACTGTAGCCACCGCA | 214 | 2842 | 2861 | 7 | | 206 |
| GGGGGCACTGTAGCCACCGC | 215 | 2843 | 2862 | 7 | | 207 |
| GGGGGGCACTGTAGCCACCG | 216 | 2844 | 2863 | 7 | | 208 |
| CCGGGTCCACTGCCCCCAGG | 217 | 2962 | 2981 | 7 | | 209 |
| TGCTGCCTCTCAGCTGCCAG | 218 | 3302 | 3321 | 7 | | 210 |
| TGCGTGCTGCCTCTCAGCTG | 219 | 3306 | 3325 | 7 | | 211 |
| CTGCTGACATCGGGGCAGAA | 220 | 3631 | 3650 | 9 | | 212 |
| CTGATGAGCTGCTGACATCG | 221 | 3639 | 3658 | 9 | | 213 |
| CAGCGCCAAGCGCTGCAGCT | 222 | 3687 | 3706 | 9 | | 214 |
| TGCTGTTCCAGCGCCAAGCG | 223 | 3695 | 3714 | 9 | | 215 |
| GGGCAGGCAGCGGCACACTG | 224 | 3745 | 3764 | 9 | | 216 |
| ACACCACATCGGGCCTTGGC | 225 | 3980 | 3999 | 10 | | 217 |
| ACCTCTGCACACCACATCGG | 226 | 3988 | 4007 | 10 | | 218 |
| GCAGGGGTGGCACACGATGC | 227 | 4177 | 4196 | 10 | | 219 |
| GGCAGGGGTGGCACACGATG | 228 | 4178 | 4197 | 10 | | 220 |
| GGGGCAGGGGTGGCACACGA | 229 | 4180 | 4199 | 10 | | 221 |
| GGGCGCGCACACAGGGATCG | 230 | 4204 | 4223 | 10 | | 222 |
| ACTCGGGCCTTCTTGGCTGT | 231 | 398 | 417 | 3 | | 223 |
| TACTCGGGCCTTCTTGGCTG | 232 | 399 | 418 | 3 | | 224 |
| TCTACTCGGGCCTTCTTGGC | 233 | 401 | 420 | 3 | | 225 |
| CTCTACTCGGGCCTTCTTGG | 234 | 402 | 421 | 3 | | 226 |
| TCCTCTACTCGGGCCTTCTT | 235 | 404 | 423 | 3 | | 227 |
| TTCCTCTACTCGGGCCTTCT | 236 | 405 | 424 | 3 | | 228 |
| GCTTCCTCTACTCGGGCCTT | 237 | 407 | 426 | 4 | | 229 |
| GGCTTCCTCTACTCGGGCCT | 238 | 408 | 427 | 4 | | 230 |
| CTAAGAGGCGGCCATAGGGA | 239 | 1579 | 1598 | 5 | | 231 |
| GCTAAGAGGCGGCCATAGGG | 240 | 1580 | 1599 | 5 | | 232 |
| TGGCTAAGAGGCGGCCATAG | 241 | 1582 | 1601 | 5 | | 233 |
| TTGGCTAAGAGGCGGCCATA | 242 | 1583 | 1602 | 5 | | 234 |
| GTGAGGTGGGGGCAGGTGTG | 243 | 1881 | 1900 | 5 | | 235 |
| TGTGAGGTGGGGGCAGGTGT | 244 | 1882 | 1901 | 5 | | 236 |
| GCTGTGAGGTGGGGGCAGGT | 245 | 1884 | 1903 | 5 | | 237 |
| GGCTGTGAGGTGGGGGCAGG | 246 | 1885 | 1904 | 5 | | 238 |
| CTGGCTGTGAGGTGGGGGCA | 247 | 1887 | 1906 | 5 | | 239 |
| GAGGAAGCCACGGTGGCAAT | 248 | 2093 | 2112 | 5 | | 240 |
| CGGCCCGCTTCTTGGCCAGC | 249 | 2614 | 2633 | 6 | | 241 |
| TCGGCCCGCTTCTTGGCCAG | 250 | 2615 | 2634 | 6 | | 242 |
| GTCGGCCCGCTTCTTGGCCA | 251 | 2616 | 2635 | 6 | | 243 |
| GGTCGGCCCGCTTCTTGGCC | 252 | 2617 | 2636 | 6 | | 244 |
| AGGTCGGCCCGCTTCTTGGC | 253 | 2618 | 2637 | 6 | | 245 |
| CAGGTCGGCCCGCTTCTTGG | 254 | 2619 | 2638 | 6 | | 246 |
| CCAGGTCGGCCCGCTTCTTG | 255 | 2620 | 2639 | 6 | | 247 |
| ACCAGGTCGGCCCGCTTCTT | 256 | 2621 | 2640 | 6 | | 248 |
| CACCAGGTCGGCCCGCTTCT | 257 | 2622 | 2641 | 6 | | 249 |
| CCACCAGGTCGGCCCGCTTC | 258 | 2623 | 2642 | 6 | | 250 |
| TCCACCAGGTCGGCCCGCTT | 259 | 2624 | 2643 | 6 | | 251 |
| CTCCACCAGGTCGGCCCGCT | 260 | 2625 | 2644 | 6 | | 252 |
| TCTCCACCAGGTCGGCCCGC | 261 | 2626 | 2645 | 6 | | 253 |
| TTCTCCACCAGGTCGGCCCG | 262 | 2627 | 2646 | 6 | | 254 |
| CTTCTCCACCAGGTCGGCCC | 263 | 2628 | 2647 | 6 | | 255 |
| ACCTTCTCCACCAGGTCGGC | 264 | 2630 | 2649 | 6 | | 256 |
| CACCTTCTCCACCAGGTCGG | 265 | 2631 | 2650 | 6 | | 257 |
| GCACCTTCTCCACCAGGTCG | 266 | 2632 | 2651 | 6 | | 258 |
| CCGCACCTTCTCCACCAGGT | 267 | 2634 | 2653 | 6 | | 259 |
| GCCGCACCTTCTCCACCAGG | 268 | 2635 | 2654 | 6 | | 260 |
| CGCCGCACCTTCTCCACCAG | 269 | 2636 | 2655 | 6 | | 261 |
| CGCGCCGCACCTTCTCCACC | 270 | 2638 | 2657 | 6 | | 262 |
| TCGCGCCGCACCTTCTCCAC | 271 | 2639 | 2658 | 6 | | 263 |
| CTCGCGCCGCACCTTCTCCA | 272 | 2640 | 2659 | 6 | | 264 |
| CCTCGCGCCGCACCTTCTCC | 273 | 2641 | 2660 | 6 | | 265 |
| GCCTCGCGCCGCACCTTCTC | 274 | 2642 | 2661 | 6 | | 266 |
| GGCCTCGCGCCGCACCTTCT | 275 | 2643 | 2662 | 6 | | 267 |
| CGGCCTCGCGCCGCACCTTC | 276 | 2644 | 2663 | 6 | | 268 |
| TCGGCCTCGCGCCGCACCTT | 277 | 2645 | 2664 | 6 | | 269 |
| CTCGGCCTCGCGCCGCACCT | 278 | 2646 | 2665 | 6 | | 270 |
| GCTCGGCCTCGCGCCGCACC | 279 | 2647 | 2666 | 6 | | 271 |
| TGCTCGGCCTCGCGCCGCAC | 280 | 2648 | 2667 | 6 | | 272 |
| GCGCTCCTTTTCTTCGCGCG | 281 | 2673 | 2692 | 6 | | 273 |
| CGCGCTCCTTTTCTTCGCGC | 282 | 2674 | 2693 | 6 | | 274 |
| TCGCGCTCCTTTTCTTCGCG | 283 | 2675 | 2694 | 6 | | 275 |
| CTCGCGCTCCTTTTCTTCGC | 284 | 2676 | 2695 | 6 | | 276 |
| GCTCGCGCTCCTTTTCTTCG | 285 | 2677 | 2696 | 6 | | 277 |
| CGCTCGCGCTCCTTTTCTTC | 286 | 2678 | 2697 | 6 | | 278 |
| GCGCTCGCGCTCCTTTTCTT | 287 | 2679 | 2698 | 6 | | 279 |
| CGCGCTCGCGCTCCTTTTCT | 288 | 2680 | 2699 | 6 | | 280 |
| TCGCGCTCGCGCTCCTTTTC | 289 | 2681 | 2700 | 6 | | 281 |
| CTCGCGCTCGCGCTCCTTTT | 290 | 2682 | 2701 | 6 | | 282 |
| GCTCGCGCTCGCGCTCCTTT | 291 | 2683 | 2702 | 6 | | 283 |
| CGCTCGCGCTCGCGCTCCTT | 292 | 2684 | 2703 | 6 | | 284 |
| CCGCTCGCGCTCGCGCTCCT | 293 | 2685 | 2704 | 6 | | 285 |
| CCCGCTCGCGCTCGCGCTCC | 294 | 2686 | 2705 | 6 | | 286 |
| TCCCGCTCGCGCTCGCGCTC | 295 | 2687 | 2706 | 6 | | 287 |
| TTCCCGCTCGCGCTCGCGCT | 296 | 2688 | 2707 | 6 | | 288 |
| GTTCCCGCTCGCGCTCGCGC | 297 | 2689 | 2708 | 6 | | 289 |
| CGTTCCCGCTCGCGCTCGCG | 298 | 2690 | 2709 | 6 | | 290 |
| GCGTTCCCGCTCGCGCTCGC | 299 | 2691 | 2710 | 6 | | 291 |
| CGCGTTCCCGCTCGCGCTCG | 300 | 2692 | 2711 | 6 | | 292 |
| TCGCGTTCCCGCTCGCGCTC | 301 | 2693 | 2712 | 6 | | 293 |
| CTCGCGTTCCCGCTCGCGCT | 302 | 2694 | 2713 | 6 | | 294 |
| TCTCGCGTTCCCGCTCGCGC | 303 | 2695 | 2714 | 6 | | 295 |
| TTCTCGCGTTCCCGCTCGCG | 304 | 2696 | 2715 | 6 | | 296 |
| TTTCTCGCGTTCCCGCTCGC | 305 | 2697 | 2716 | 6 | | 297 |
| CTTTCTCGCGTTCCCGCTCG | 306 | 2698 | 2717 | 6 | | 298 |
| CTCGCGCTCCTTCTCGCGCT | 307 | 2724 | 2743 | 6 | | 299 |
| GCTCGCGCTCCTTCTCGCGC | 308 | 2725 | 2744 | 6 | | 300 |
| AAGCTCGCGCTCCTTCTCGC | 309 | 2727 | 2746 | 6 | | 301 |
| CAAGCTCGCGCTCCTTCTCG | 310 | 2728 | 2747 | 6 | | 302 |
| TTCAAGCTCGCGCTCCTTCT | 311 | 2730 | 2749 | 6 | | 303 |
| GTTCAAGCTCGCGCTCCTTC | 312 | 2731 | 2750 | 6 | | 304 |
| CGTTCAAGCTCGCGCTCCTT | 313 | 2732 | 2751 | 6 | | 305 |
| GCGTTCAAGCTCGCGCTCCT | 314 | 2733 | 2752 | 6 | | 306 |
| CCGCACTGCCCGGTTCAAAT | 315 | 2827 | 2846 | 7 | | 307 |
| ACCGCACTGCCCGGTTCAAA | 316 | 2828 | 2847 | 7 | | 308 |
| CCACCGCACTGCCCGGTTCA | 317 | 2830 | 2849 | 7 | | 309 |
| GCCACCGCACTGCCCGGTTC | 318 | 2831 | 2850 | 7 | | 310 |
| TAGCCACCGCACTGCCCGGT | 319 | 2833 | 2852 | 7 | | 311 |
| GTAGCCACCGCACTGCCCGG | 320 | 2834 | 2853 | 7 | | 312 |
| CTGTAGCCACCGCACTGCCC | 321 | 2836 | 2855 | 7 | | 313 |
| ACTGTAGCCACCGCACTGCC | 322 | 2837 | 2856 | 7 | | 314 |
| CCCGGGTCCACTGCCCCCAG | 323 | 2963 | 2982 | 7 | | 315 |
| GTGCTGCCTCTCAGCTGCCA | 324 | 3303 | 3322 | 7 | | 316 |
| CGTGCTGCCTCTCAGCTGCC | 325 | 3304 | 3323 | 7 | | 317 |
| GCGTGCTGCCTCTCAGCTGC | 326 | 3305 | 3324 | 7 | | 318 |
| CTGCGTGCTGCCTCTCAGCT | 327 | 3307 | 3326 | 7 | | 319 |
| TCTGCGTGCTGCCTCTCAGC | 328 | 3308 | 3327 | 7 | | 320 |
| TTCTGCGTGCTGCCTCTCAG | 329 | 3309 | 3328 | 7 | | 321 |
| TTTCTGCGTGCTGCCTCTCA | 330 | 3310 | 3329 | 7 | | 322 |
| CTTTCTGCGTGCTGCCTCTC | 331 | 3311 | 3330 | 7 | | 323 |
| CCTTTCTGCGTGCTGCCTCT | 332 | 3312 | 3331 | 7 | | 324 |
| CCCTTTCTGCGTGCTGCCTC | 333 | 3313 | 3332 | 7 | | 325 |
| ACCCTTTCTGCGTGCTGCCT | 334 | 3314 | 3333 | 7 | | 326 |
| CACCCTTTCTGCGTGCTGCC | 335 | 3315 | 3334 | 7 | | 327 |
| CCACCCTTTCTGCGTGCTGC | 336 | 3316 | 3335 | 7 | | 328 |
| GCCACCCTTTCTGCGTGCTG | 337 | 3317 | 3336 | 7 | | 329 |
| CGCCACCCTTTCTGCGTGCT | 338 | 3318 | 3337 | 7 | | 330 |
| CCGCCACCCTTTCTGCGTGC | 339 | 3319 | 3338 | 7 | | 331 |
| GCCGCCACCCTTTCTGCGTG | 340 | 3320 | 3339 | 7 | | 332 |
| GGCCGCCACCCTTTCTGCGT | 341 | 3321 | 3340 | 7 | | 333 |
| GGGCCGCCACCCTTTCTGCG | 342 | 3322 | 3341 | 7 | | 334 |
| AGGGCCGCCACCCTTTCTGC | 343 | 3323 | 3342 | 7 | | 335 |
| CAGGGCCGCCACCCTTTCTG | 344 | 3324 | 3343 | 7 | | 336 |
| GGCAGAAAGGGAGGCCGGCA | 345 | 3618 | 3637 | 9 | | 337 |
| GGGCAGAAAGGGAGGCCGGC | 346 | 3619 | 3638 | 9 | | 338 |
| GGGGCAGAAAGGGAGGCCGG | 347 | 3620 | 3639 | 9 | | 339 |
| CGGGGCAGAAAGGGAGGCCG | 348 | 3621 | 3640 | 9 | | 340 |
| TCGGGGCAGAAAGGGAGGCC | 349 | 3622 | 3641 | 9 | | 341 |
| ATCGGGGCAGAAAGGGAGGC | 350 | 3623 | 3642 | 9 | | 342 |
| CATCGGGGCAGAAAGGGAGG | 351 | 3624 | 3643 | 9 | | 343 |
| ACATCGGGGCAGAAAGGGAG | 352 | 3625 | 3644 | 9 | | 344 |
| GACATCGGGGCAGAAAGGGA | 353 | 3626 | 3645 | 9 | | 345 |
| TGACATCGGGGCAGAAAGGG | 354 | 3627 | 3646 | 9 | | 346 |
| CTGACATCGGGGCAGAAAGG | 355 | 3628 | 3647 | 9 | | 347 |
| GCTGACATCGGGGCAGAAAG | 356 | 3629 | 3648 | 9 | | 348 |
| TGCTGACATCGGGGCAGAAA | 357 | 3630 | 3649 | 9 | | 349 |
| GCTGCTGACATCGGGGCAGA | 358 | 3632 | 3651 | 9 | | 350 |
| AGCTGCTGACATCGGGGCAG | 359 | 3633 | 3652 | 9 | | 351 |
| GAGCTGCTGACATCGGGGCA | 360 | 3634 | 3653 | 9 | | 352 |
| ATGAGCTGCTGACATCGGGG | 361 | 3636 | 3655 | 9 | | 353 |
| GATGAGCTGCTGACATCGGG | 362 | 3637 | 3656 | 9 | | 354 |
| TGATGAGCTGCTGACATCGG | 363 | 3638 | 3657 | 9 | | 355 |
| GCTGATGAGCTGCTGACATC | 364 | 3640 | 3659 | 9 | | 356 |
| AGCTGATGAGCTGCTGACAT | 365 | 3641 | 3660 | 9 | | 357 |
| CAGCTGATGAGCTGCTGACA | 366 | 3642 | 3661 | 9 | | 358 |
| GCAGCTGATGAGCTGCTGAC | 367 | 3643 | 3662 | 9 | | 359 |
| TGCAGCTGATGAGCTGCTGA | 368 | 3644 | 3663 | 9 | | 360 |
| CTGCAGCTGATGAGCTGCTG | 369 | 3645 | 3664 | 9 | | 361 |
| CCTGCAGCTGATGAGCTGCT | 370 | 3646 | 3665 | 9 | | 362 |
| GCCTGCAGCTGATGAGCTGC | 371 | 3647 | 3666 | 9 | | 363 |
| GGCCTGCAGCTGATGAGCTG | 372 | 3648 | 3667 | 9 | | 364 |
| TGGCCTGCAGCTGATGAGCT | 373 | 3649 | 3668 | 9 | | 365 |
| ATGGCCTGCAGCTGATGAGC | 374 | 3650 | 3669 | 9 | | 366 |
| CATGGCCTGCAGCTGATGAG | 375 | 3651 | 3670 | 9 | | 367 |
| GCATGGCCTGCAGCTGATGA | 376 | 3652 | 3671 | 9 | | 368 |
| TGCATGGCCTGCAGCTGATG | 377 | 3653 | 3672 | 9 | | 369 |
| CAGCTCAGCTGACTGTGCGT | 378 | 3672 | 3691 | 9 | | 370 |
| GCAGCTCAGCTGACTGTGCG | 379 | 3673 | 3692 | 9 | | 371 |
| TGCAGCTCAGCTGACTGTGC | 380 | 3674 | 3693 | 9 | | 372 |
| CTGCAGCTCAGCTGACTGTG | 381 | 3675 | 3694 | 9 | | 373 |
| GCTGCAGCTCAGCTGACTGT | 382 | 3676 | 3695 | 9 | | 374 |
| CGCTGCAGCTCAGCTGACTG | 383 | 3677 | 3696 | 9 | | 375 |
| GCGCTGCAGCTCAGCTGACT | 384 | 3678 | 3697 | 9 | | 376 |
| AGCGCTGCAGCTCAGCTGAC | 385 | 3679 | 3698 | 9 | | 377 |
| AAGCGCTGCAGCTCAGCTGA | 386 | 3680 | 3699 | 9 | | 378 |
| CAAGCGCTGCAGCTCAGCTG | 387 | 3681 | 3700 | 9 | | 379 |
| CCAAGCGCTGCAGCTCAGCT | 388 | 3682 | 3701 | 9 | | 380 |
| GCCAAGCGCTGCAGCTCAGC | 389 | 3683 | 3702 | 9 | | 381 |
| CGCCAAGCGCTGCAGCTCAG | 390 | 3684 | 3703 | 9 | | 382 |
| GCGCCAAGCGCTGCAGCTCA | 391 | 3685 | 3704 | 9 | | 383 |
| AGCGCCAAGCGCTGCAGCTC | 392 | 3686 | 3705 | 9 | | 384 |
| CCAGCGCCAAGCGCTGCAGC | 393 | 3688 | 3707 | 9 | | 385 |
| TCCAGCGCCAAGCGCTGCAG | 394 | 3689 | 3708 | 9 | | 386 |
| TTCCAGCGCCAAGCGCTGCA | 395 | 3690 | 3709 | 9 | | 387 |
| TGTTCCAGCGCCAAGCGCTG | 396 | 3692 | 3711 | 9 | | 388 |
| CTGTTCCAGCGCCAAGCGCT | 397 | 3693 | 3712 | 9 | | 389 |
| GCTGTTCCAGCGCCAAGCGC | 398 | 3694 | 3713 | 9 | | 390 |
| CTGCTGTTCCAGCGCCAAGC | 399 | 3696 | 3715 | 9 | | 391 |
| GCTGCTGTTCCAGCGCCAAG | 400 | 3697 | 3716 | 9 | | 392 |
| TGCTGCTGTTCCAGCGCCAA | 401 | 3698 | 3717 | 9 | | 393 |
| CTGCTGCTGTTCCAGCGCCA | 402 | 3699 | 3718 | 9 | | 394 |
| ACTGCTGCTGTTCCAGCGCC | 403 | 3700 | 3719 | 9 | | 395 |
| CACTGCTGCTGTTCCAGCGC | 404 | 3701 | 3720 | 9 | | 396 |
| CCACTGCTGCTGTTCCAGCG | 405 | 3702 | 3721 | 9 | | 397 |
| GCCACTGCTGCTGTTCCAGC | 406 | 3703 | 3722 | 9 | | 398 |
| AGCCACTGCTGCTGTTCCAG | 407 | 3704 | 3723 | 9 | | 399 |
| CAGCCACTGCTGCTGTTCCA | 408 | 3705 | 3724 | 9 | | 400 |
| GCAGCCACTGCTGCTGTTCC | 409 | 3706 | 3725 | 9 | | 401 |
| TGCAGCCACTGCTGCTGTTC | 410 | 3707 | 3726 | 9 | | 402 |
| ATGCAGCCACTGCTGCTGTT | 411 | 3708 | 3727 | 9 | | 403 |
| GGCAGCGGCACACTGTGCAG | 412 | 3740 | 3759 | 9 | | 404 |
| AGGCAGCGGCACACTGTGCA | 413 | 3741 | 3760 | 9 | | 405 |
| CAGGCAGCGGCACACTGTGC | 414 | 3742 | 3761 | 9 | | 406 |
| GCAGGCAGCGGCACACTGTG | 415 | 3743 | 3762 | 9 | | 407 |
| GGCAGGCAGCGGCACACTGT | 416 | 3744 | 3763 | 9 | | 408 |
| TTGGCCTTCTGTCTGTCCCT | 417 | 3965 | 3984 | 10 | | 409 |
| CTTGGCCTTCTGTCTGTCCC | 418 | 3966 | 3985 | 10 | | 410 |
| CCTTGGCCTTCTGTCTGTCC | 419 | 3967 | 3986 | 10 | | 411 |
| GCCTTGGCCTTCTGTCTGTC | 420 | 3968 | 3987 | 10 | | 412 |
| GGCCTTGGCCTTCTGTCTGT | 421 | 3969 | 3988 | 10 | | 413 |
| GGGCCTTGGCCTTCTGTCTG | 422 | 3970 | 3989 | 10 | | 414 |
| CGGGCCTTGGCCTTCTGTCT | 423 | 3971 | 3990 | 10 | | 415 |
| TCGGGCCTTGGCCTTCTGTC | 424 | 3972 | 3991 | 10 | | 416 |
| ATCGGGCCTTGGCCTTCTGT | 425 | 3973 | 3992 | 10 | | 417 |
| CATCGGGCCTTGGCCTTCTG | 426 | 3974 | 3993 | 10 | | 418 |
| ACATCGGGCCTTGGCCTTCT | 427 | 3975 | 3994 | 10 | | 419 |
| CACATCGGGCCTTGGCCTTC | 428 | 3976 | 3995 | 10 | | 420 |
| CCACATCGGGCCTTGGCCTT | 429 | 3977 | 3996 | 10 | | 421 |
| ACCACATCGGGCCTTGGCCT | 430 | 3978 | 3997 | 10 | | 422 |
| CACCACATCGGGCCTTGGCC | 431 | 3979 | 3998 | 10 | | 423 |
| CACACCACATCGGGCCTTGG | 432 | 3981 | 4000 | 10 | | 424 |
| GCACACCACATCGGGCCTTG | 433 | 3982 | 4001 | 10 | | 425 |
| TGCACACCACATCGGGCCTT | 434 | 3983 | 4002 | 10 | | 426 |
| TCTGCACACCACATCGGGCC | 435 | 3985 | 4004 | 10 | | 427 |
| CTCTGCACACCACATCGGGC | 436 | 3986 | 4005 | 10 | | 428 |
| CCTCTGCACACCACATCGGG | 437 | 3987 | 4006 | 10 | | 429 |
| CACCTCTGCACACCACATCG | 438 | 3989 | 4008 | 10 | | 430 |
| CCACCTCTGCACACCACATC | 439 | 3990 | 4009 | 10 | | 431 |
| CCCACCTCTGCACACCACAT | 440 | 3991 | 4010 | 10 | | 432 |
| CCCCACCTCTGCACACCACA | 441 | 3992 | 4011 | 10 | | 433 |
| TCCCCACCTCTGCACACCAC | 442 | 3993 | 4012 | 10 | | 434 |
| CTCCCCACCTCTGCACACCA | 443 | 3994 | 4013 | 10 | | 435 |
| CCTCCCCACCTCTGCACACC | 444 | 3995 | 4014 | 10 | | 436 |
| ACCTCCCCACCTCTGCACAC | 445 | 3996 | 4015 | 10 | | 437 |
| CACCTCCCCACCTCTGCACA | 446 | 3997 | 4016 | 10 | | 438 |
| CCACCTCCCCACCTCTGCAC | 447 | 3998 | 4017 | 10 | | 439 |
| AGGGGCAGGGGTGGCACACG | 448 | 4181 | 4200 | 10 | | 440 |
| GAGGGGCAGGGGTGGCACAC | 449 | 4182 | 4201 | 10 | | 441 |
| GGAGGGGCAGGGGTGGCACA | 450 | 4183 | 4202 | 10 | | 442 |
| GGGAGGGGCAGGGGTGGCAC | 451 | 4184 | 4203 | 10 | | 443 |
| GGGGAGGGGCAGGGGTGGCA | 452 | 4185 | 4204 | 10 | | 444 |
| CGGGGAGGGGCAGGGGTGGC | 453 | 4186 | 4205 | 10 | | 445 |
| TCGGGGAGGGGCAGGGGTGG | 454 | 4187 | 4206 | 10 | | 446 |
| ATCGGGGAGGGGCAGGGGTG | 455 | 4188 | 4207 | 10 | | 447 |
| GATCGGGGAGGGGCAGGGGT | 456 | 4189 | 4208 | 10 | | 448 |
| GGATCGGGGAGGGGCAGGGG | 457 | 4190 | 4209 | 10 | | 449 |
| GGGATCGGGGAGGGGCAGGG | 458 | 4191 | 4210 | 10 | | 450 |
| AGGGATCGGGGAGGGGCAGG | 459 | 4192 | 4211 | 10 | | 451 |
| CAGGGATCGGGGAGGGGCAG | 460 | 4193 | 4212 | 10 | | 452 |
| ACAGGGATCGGGGAGGGGCA | 461 | 4194 | 4213 | 10 | | 453 |
| CACAGGGATCGGGGAGGGGC | 462 | 4195 | 4214 | 10 | | 454 |
| ACACAGGGATCGGGGAGGGG | 463 | 4196 | 4215 | 10 | | 455 |
| CACACAGGGATCGGGGAGGG | 464 | 4197 | 4216 | 10 | | 456 |
| GCACACAGGGATCGGGGAGG | 465 | 4198 | 4217 | 10 | | 457 |
| CGCACACAGGGATCGGGGAG | 466 | 4199 | 4218 | 10 | | 458 |
| GCGCACACAGGGATCGGGGA | 467 | 4200 | 4219 | 10 | | 459 |
| CGCGCACACAGGGATCGGGG | 468 | 4201 | 4220 | 10 | | 460 |
| GCGCGCACACAGGGATCGGG | 469 | 4202 | 4221 | 10 | | 461 |
| GGCGCGCACACAGGGATCGG | 470 | 4203 | 4222 | 10 | | 462 |
| GGGGCGCGCACACAGGGATC | 471 | 4205 | 4224 | 10 | | 463 |
| GGGGGCGCGCACACAGGGAT | 472 | 4206 | 4225 | 10 | | 464 |

In Table 3, all oligonucleotides are 5-10-5 gapmers having a 2′-O-MOE group, bonds between nucleosides in the 5′ wing region and the 3′ wing region are phosphorothioate (P═S) or phosphodiester (P═O), and bonds between nucleosides in the gap region are all phosphorothioate (P═S). In Table 3, each phosphodiester (P═O) bond is shown as “\hat{ }”. Besides, C in the gap region and the wing region is actually methyl C, and T in the wing region is actually T.

TABLE 3
Table 3 Gapmer (oligonucleotide) to be tested containing
phosphodiester bond in bonds between wing nucleosides

| | | Start position | End position | | | SEQ |
|---|---|---|---|---|---|---|
| ASO base sequence | ASO | in SEQ ID | in SEQ ID | Target | ASO | ID |
| (DNA) 5′ to 3′ | No. | NO: 471 | NO: 471 | exon | name | NO: |
| AG^{∧}A^{∧}G^{∧}ACCTGGTCCAAG^{∧}A^{∧}T^{∧}TC | 473 | 4043 | 4062 | 10 | DRA-274 | 472 |
| AG^{∧}A^{∧}GACCTGGTCCAAGA^{∧}T^{∧}TC | 474 | 4043 | 4062 | 10 | DRA-275 | 472 |
| AG^{∧}AG^{∧}ACCTGGTCCAAG^{∧}AT^{∧}TC | 475 | 4043 | 4062 | 10 | DRA-276 | 472 |
| AGA^{∧}G^{∧}ACCTGGTCCAAG^{∧}A^{∧}TTC | 476 | 4043 | 4062 | 10 | DRA-277 | 472 |
| AG^{∧}AGACCTGGTCCAAGAT^{∧}TC | 477 | 4043 | 4062 | 10 | DRA-278 | 472 |
| AGA^{∧}GACCTGGTCCAAGA^{∧}TTC | 478 | 4043 | 4062 | 10 | DRA-279 | 472 |
| AGAG^{∧}ACCTGGTCCAAG^{∧}ATTC | 479 | 4043 | 4062 | 10 | DRA-280 | 472 |
| GA^{∧}T^{∧}G^{∧}GGCATTGCTGTT^{∧}G^{∧}G^{∧}CT | 480 | 1597 | 1616 | 5 | DRA-281 | 177 |
| GA^{∧}T^{∧}GGGCATTGCTGTTG^{∧}G^{∧}CT | 481 | 1597 | 1616 | 5 | DRA-282 | 177 |
| GA^{∧}TG^{∧}GGCATTGCTGTT^{∧}GG^{∧}CT | 482 | 1597 | 1616 | 5 | DRA-283 | 177 |
| GAT^{∧}G^{∧}GGCATTGCTGTT^{∧}G^{∧}GCT | 483 | 1597 | 1616 | 5 | DRA-284 | 177 |
| GA^{∧}TGGGCATTGCTGTTGG^{∧}CT | 484 | 1597 | 1616 | 5 | DRA-285 | 177 |
| GAT^{∧}GGGCATTGCTGTTG^{∧}GCT | 485 | 1597 | 1616 | 5 | DRA-286 | 177 |
| GATG^{∧}GGCATTGCTGTT^{∧}GGCT | 486 | 1597 | 1616 | 5 | DRA-287 | 177 |
| TC^{∧}C^{∧}T^{∧}TCTCGCGCTCGC^{∧}G^{∧}C^{∧}TC | 487 | 2717 | 2736 | 6 | DRA-298 | 133 |
| TC^{∧}C^{∧}TTCTCGCGCTCGCG^{∧}C^{∧}TC | 488 | 2717 | 2736 | 6 | DRA-308 | 133 |
| TC^{∧}CT^{∧}TCTCGCGCTCGC^{∧}GC^{∧}TC | 489 | 2717 | 2736 | 6 | DRA-299 | 133 |
| TCC^{∧}T^{∧}TCTCGCGCTCGC^{∧}G^{∧}CTC | 490 | 2717 | 2736 | 6 | DRA-309 | 133 |
| TC^{∧}CTTCTCGCGCTCGCGC^{∧}TC | 491 | 2717 | 2736 | 6 | DRA-310 | 133 |
| TCC^{∧}TTCTCGCGCTCGCG^{∧}CTC | 492 | 2717 | 2736 | 6 | DRA-311 | 133 |
| TCCT^{∧}TCTCGCGCTCGC^{∧}GCTC | 493 | 2717 | 2736 | 6 | DRA-312 | 133 |
| CT^{∧}C^{∧}G^{∧}CGCTCGCGCTCT^{∧}T^{∧}T^{∧}CT | 494 | 2712 | 2731 | 6 | DRA-300 | 196 |
| CT^{∧}CG^{∧}CGCTCGCGCTCT^{∧}TT^{∧}CT | 495 | 2712 | 2731 | 6 | DRA-301 | 196 |
| GC^{∧}T^{∧}G^{∧}TTGGCTAAGAGG^{∧}C^{∧}G^{∧}GC | 496 | 1587 | 1606 | 5 | DRA-302 | 170 |

\hat{ }: Phosphodiester bond

In Table 4, oligonucleotides are all 5-10-5 gapmers having a 2′-O-MOE group, bonds between nucleosides in the 5′ wing region and the 3′ wing region are phosphorothioate (P═S) or phosphodiester (P═O), and bonds between nucleosides in the gap region are all phosphorothioate (P═S). In Table 4, each phosphodiester (P═O) bond is shown as “\hat{ }”. Besides, C in the gap region and the wing region is actually methyl C, T in the wing region is actually T, and underlined bases of the 3′ wing region are bases different from those of SEQ ID NO: 196.

TABLE 4
Table 4 Gapmer (oligonucleotide) to
be tested different in one nucleotide in
3′ wing from SEQ ID NO: 196

| | | Start | End | | | |
|---|---|---|---|---|---|---|
| ASO base | | position | position | | | |
| sequence | | in | in | | | SEQ |
| (DNA) | ASO | SEQ ID | SEQ ID | Target | ASO | ID |
| 5′ to 3′ | No. | NO: 471 | NO: 471 | exon | name | NO: |
| CTCGCGCTCGCG | 497 | 2712 | 2731 | 10 | DRA-303 | 473 |
| CTCCTTCT | | | | | | |
| CT^{∧}C^{∧}G^{∧}CGCTCG | 498 | 2712 | 2731 | 10 | DRA-304 | 473 |
| CGCTCC^{∧}T^{∧}T^{∧}CT | | | | | | |
| CT^{∧}CG^{∧}CGCTCG | 499 | 2712 | 2731 | 10 | DRA-305 | 473 |
| CGCTCC^{∧}TT^{∧}CT | | | | | | |

\hat{ }: Phosphodiester bond
underlined: bases different from SEQ ID NO: 196

### [Test Example 1] Measurement of ATN-1 Gene Knockdown Activity (Inhibitory Activity) of Gapmer Using A204 Human Rhabdomyosarcoma Cell Line

Into 3.0×10⁵ A204 cells (obtained from ATCC), each of gapmers to be tested shown in Tables 1 and 2, or a positive control gapmer was introduced in a concentration of 1 μM or 2 μM with 4D-Nucleofector™ using SF Cell Line 4D-Nucleotector™ X KitS in accordance with the protocol attached to the kit. DS-130 was used as the program. After the introduction, the cells were cultured overnight in a 10% fetal bovine serum (FBS) (NICHIREI) and McCoy's 5A Medium (SIGMA) containing 1 mM L-glutamine (Gibco) under conditions of 37° C. and 5% CO₂. As the positive control gapmer, OMe-6 (5′-AGAGACCTGGTCCAAGATTC-3′, SEQ ID NO: 472) having a 2′-OMe group in a nucleoside of the wing, or NRH-71 (5′-AGAGACCTGGTCCAAGATTC-3′, SEQ ID NO: 472) having a 2′-O-MOE group in a nucleoside of the wing, both of which had been confirmed to have activity through a preliminary test (data not shown), was used. Both the oligonucleotides were 5-10-5 gapmers, and bonds between nucleosides were all phosphorothioate (P═S). Besides, in OMe-6, T (thymine) in the wing region was actually U (uracil). In NRH-71, C (cytosine) in the gap region and the wing region was actually methyl C, and T in the wing region was actually T. These positive control gapmers were synthesized in the same manner as the gapmers to be tested. On the next day, after washing the resultant cells with PBS (Nacalai Tesque, Inc.), RA1 (Takara Bio Inc.) containing 1% 2-mercaptoethanol (Nacalai Tesque, Inc.) was added thereto to dissolve the cells, which were collected on NucleoSpin® Filter (Takara Bio Inc.). After removing an insoluble fraction by centrifugation, total RNA was extracted in accordance with the protocol attached to NucleoSpin® RNA (Takara Bio Inc.). The concentration of the extracted total RNA was measured with NanoDrop (Thermo Fisher). 200 ng of the extracted total RNA was subjected to a reverse transcription (RT) reaction with High Capacity cDNA Reverse Transcription Kit (Applied Biosystems) and a random primer attached to the kit. Specifically, in accordance with the protocol attached to the kit, a reaction solution was prepared. TaKaRa PCR Thermal Cycler Dice Touch (Takara Bio Inc.) was used as the thermal cycler. The program of the RT reaction used was as follows.
25° C., 10 minutes: primer annealing
37° C., 120 minutes: reverse transcription reaction
85° C., 5 minutes: reverse transcriptase inactivation

Thereafter, qPCR was performed with Fast SYBR Green Master Mix (Thermo Fisher Scientific) using the RT reaction solution as a template, and the ATN1 RNA expression level endogenously expressed in the A204 cells was measured. In the qPCR reaction, sequences of 2 portions in ATN1 (exon 4 to exon 5, and exon 6 to exon 7) were used as templates. Specifically, in accordance with the protocol attached to the kit, a reaction solution was prepared. For the qPCR, QuantStudio 6 Flex Real-Time PCR System (Applied Biosystems) was used. The program of the qPCR used was as follows.
50° C., 2 minutes; and 95° C., 10 minutes: polymerase activation, cDNA thermal denaturation
[95° C., 15 seconds; and 60° C., 1 minute]×40 cycles: PCR Amplification
95° C., 15 seconds; and 60° C., 1 minute to 95° C., 15 seconds (Melting Curve 0.05° C./s): melting curve analysis

Primers used in the detection of the ATN1 RNA are shown in Table 4. The ATN1 RNA expression level was corrected with β-actin RNA expression level of house keeping gene. Primers used in the detection of β-actin RNA are shown in Table 4. An inhibition ratio of ATN1 expression of the gapmers to the ATN1 RNA expression level of cells having been subjected to the introduction operation only with 4D-Nucleofector™ without adding the gapmers was calculated based on a ratio of the ATN1 RNA expression level obtained by introducing the gapmers to be tested, and thus, the inhibitory activity of the gapmers to be tested was analyzed. Results are shown in Tables 5 to 9.

TABLE 5
Table 5-1 Endogenous ATN1 Primer used
for RNA detection and recognized exon site

| | Primer set for detection | | | |
|---|---|---|---|---|
| | | SEQ | Reverse | SEQ |
| Recognized | Sense primer | ID | primer | ID |
| exon | 5′ to 3′ | NO: | 5′ to 3′ | NO: |
| exon4-5 | TCAACAAGCAGGG | 465 | CTTCGGTT | 467 |
| | TCGGAGT | | GTCCTGGT | |
| | | | CGATA | |
| exon6-7 | TTGAACGCAGCGT | 466 | TTGCGATT | 468 |
| | GAAGTTG | | GCCAGGAG | |
| | | | ACAT | |

Table 5-2 β-Actin used as gene for
correction Primer used for RNA detection

| | Primer set for detection | | | |
|---|---|---|---|---|
| | | SEQ | Reverse | SEQ |
| Gene for | Sense primer | ID | primer | ID |
| correction | 5′ to 3′ | NO: | 5′ to 3′ | NO: |
| β-actin | GAAGATCAAGATC | 469 | TACTCCTG | 470 |
| | ATTGCTCCT | | CTTGCTGA | |
| | | | TCCA | |

TABLE 6
Ratio of ATN-1 gene expression level in A204 cell administered with
gapmer (2.0 μM) having 2′-OMe group in wing nucleoside

| | | Ratio of | | Ratio of | Average | |
|---|---|---|---|---|---|---|
| | | mRNA | | mRNA | of | |
| | mRNA | expression | mRNA | expression | ratio of | |
| | expression | level/ | expression | level/ | mRNA | SEQ |
| ASO | level | OMe-6 | level | OMe-6 | expression | ID |
| No. | (exon4-5) | (exon4-5) | (exon6-7) | (exon6-7) | level | NO: |
| 1 | 0.94 | 2.09 | 1.00 | 2.29 | 2.19 | 1 |
| 2 | 0.97 | 2.15 | 1.00 | 2.30 | 2.23 | 2 |
| 3 | 0.75 | 1.64 | 0.76 | 1.61 | 1.62 | 3 |
| 4 | 0.44 | 1.27 | 0.47 | 1.28 | 1.27 | 4 |
| 6 | 0.61 | 1.56 | 0.69 | 1.60 | 1.58 | 6 |
| 7 | 0.11 | 0.26 | 0.14 | 0.30 | 0.28 | 7 |
| 8 | 0.66 | 1.90 | 0.67 | 1.80 | 1.85 | 8 |
| 9 | 0.33 | 0.73 | 0.40 | 0.80 | 0.76 | 9 |
| 10 | 0.20 | 0.47 | 0.24 | 0.52 | 0.49 | 10 |
| 11 | 0.30 | 0.77 | 0.34 | 0.78 | 0.77 | 11 |
| 12 | 0.15 | 0.35 | 0.18 | 0.39 | 0.37 | 12 |
| 13 | 0.18 | 0.46 | 0.17 | 0.39 | 0.42 | 13 |
| 14 | 0.75 | 1.59 | 0.83 | 1.69 | 1.64 | 14 |
| 15 | 0.62 | 1.37 | 0.53 | 1.23 | 1.30 | 15 |
| 16 | 0.47 | 1.03 | 0.40 | 0.93 | 0.98 | 16 |
| 17 | 0.39 | 0.71 | 0.40 | 0.80 | 0.76 | 17 |
| 18 | 0.72 | 1.61 | 0.67 | 1.54 | 1.57 | 18 |
| 19 | 0.74 | 1.33 | 0.77 | 1.37 | 1.35 | 19 |
| 20 | 0.70 | 1.01 | 0.72 | 1.27 | 1.14 | 20 |
| 21 | 0.71 | 1.50 | 0.84 | 1.71 | 1.60 | 21 |
| 22 | 0.29 | 0.82 | 0.29 | 0.77 | 0.80 | 22 |
| 23 | 0.78 | 1.65 | 0.85 | 1.73 | 1.69 | 23 |
| 24 | 0.52 | 1.09 | 0.62 | 1.26 | 1.17 | 24 |
| 25 | 0.16 | 0.41 | 0.17 | 0.38 | 0.40 | 25 |
| 26 | 0.59 | 1.50 | 0.63 | 1.44 | 1.47 | 26 |
| 27 | 0.18 | 0.42 | 0.18 | 0.39 | 0.41 | 27 |
| 28 | 0.19 | 0.49 | 0.21 | 0.48 | 0.49 | 28 |
| 29 | 0.39 | 1.10 | 0.47 | 1.27 | 1.19 | 29 |
| 30 | 0.11 | 0.32 | 0.13 | 0.34 | 0.33 | 30 |
| 31 | 0.43 | 0.94 | 0.49 | 1.02 | 0.98 | 31 |
| 32 | 0.79 | 2.28 | 0.76 | 2.05 | 2.16 | 32 |
| 33 | 0.69 | 1.51 | 0.71 | 1.49 | 1.50 | 33 |
| 34 | 0.79 | 1.72 | 0.92 | 1.93 | 1.82 | 34 |
| 35 | 0.79 | 1.72 | 0.89 | 1.87 | 1.80 | 35 |
| 36 | 0.84 | 1.82 | 0.80 | 1.68 | 1.75 | 36 |
| 37 | 0.74 | 1.62 | 0.77 | 1.62 | 1.62 | 37 |
| 39 | 0.75 | 1.63 | 0.84 | 1.76 | 1.69 | 39 |
| 40 | 0.82 | 2.35 | 0.80 | 2.15 | 2.25 | 40 |
| 41 | 0.64 | 1.39 | 0.55 | 1.15 | 1.27 | 41 |
| 42 | 0.58 | 1.66 | 0.53 | 1.43 | 1.54 | 42 |
| 43 | 0.80 | 1.75 | 0.90 | 1.90 | 1.82 | 43 |
| 44 | 0.61 | 1.33 | 0.63 | 1.47 | 1.40 | 44 |
| 45 | 0.38 | 1.09 | 0.36 | 0.96 | 1.03 | 45 |
| 46 | 0.92 | 2.00 | 0.92 | 2.14 | 2.07 | 46 |
| 47 | 0.86 | 1.87 | 0.86 | 2.00 | 1.94 | 47 |
| 48 | 0.38 | 1.09 | 0.44 | 1.18 | 1.13 | 48 |
| 49 | 0.56 | 1.21 | 0.53 | 1.23 | 1.22 | 49 |
| 50 | 0.72 | 1.56 | 0.69 | 1.60 | 1.58 | 50 |
| 52 | 0.44 | 1.03 | 0.47 | 1.08 | 1.06 | 52 |
| 53 | 0.80 | 2.03 | 0.83 | 1.90 | 1.97 | 53 |
| 54 | 0.75 | 1.63 | 0.78 | 1.81 | 1.72 | 54 |
| 55 | 0.77 | 1.67 | 0.76 | 1.78 | 1.72 | 55 |
| 56 | 0.62 | 1.34 | 0.63 | 1.46 | 1.40 | 56 |
| 57 | 0.98 | 2.14 | 1.00 | 2.47 | 2.30 | 57 |
| 58 | 0.68 | 1.26 | 0.70 | 1.21 | 1.23 | 58 |

TABLE 7
Ratio of ATN-1 gene expression level in A204 cell administered with
gapmer (1.0 μM) having 2′-OMe group in wing nucleoside

| | | Ratio of | | Ratio of | Average | |
|---|---|---|---|---|---|---|
| | | mRNA | | mRNA | of | |
| | mRNA | expression | mRNA | expression | ratio of | |
| | expression | level/ | expression | level/ | mRNA | SEQ |
| ASO | level | OMe-6 | level | OMe-6 | expression | ID |
| No. | (exon4-5) | (exon4-5) | (exon6-7) | (exon6-7) | level | NO: |
| 1 | 0.91 | 1.25 | 1.00 | 1.21 | 1.23 | 1 |
| 2 | 0.88 | 1.21 | 1.00 | 1.22 | 1.21 | 2 |
| 7 | 0.15 | 0.30 | 0.15 | 0.29 | 0.30 | 7 |
| 10 | 0.23 | 0.46 | 0.25 | 0.49 | 0.47 | 10 |
| 12 | 0.21 | 0.42 | 0.21 | 0.40 | 0.41 | 12 |
| 27 | 0.25 | 0.48 | 0.25 | 0.48 | 0.48 | 27 |

TABLE 8
Ratio of ATN-1 gene expression level in A204
cell administered with gapmer (2.0 μM) having
2′-O—MOE group in wing nucleoside

| | | Ratio of | | Ratio of | Average | |
|---|---|---|---|---|---|---|
| | | mRNA | | mRNA | of | |
| | mRNA | expression | mRNA | expression | ratio of | |
| | expression | level/ | expression | level/ | mRNA | SEQ |
| ASO | level | NRH-71 | level | NRH-71 | expression | ID |
| No. | (exon4-5) | (exon4-5) | (exon6-7) | (exon6-7) | level | NO: |
| 59 | 1.00 | 3.18 | 0.88 | 2.44 | 2.81 | 59 |
| 60 | 0.23 | 0.46 | 0.23 | 0.58 | 0.52 | 60 |
| 61 | 0.04 | 0.11 | 0.07 | 0.20 | 0.15 | 61 |
| 64 | 0.04 | 0.12 | 0.13 | 0.35 | 0.23 | 64 |
| 65 | 0.11 | 0.34 | 0.22 | 0.62 | 0.48 | 65 |
| 69 | 0.05 | 0.18 | 0.04 | 0.24 | 0.21 | 69 |
| 71 | 0.11 | 0.32 | 0.12 | 0.34 | 0.33 | 71 |
| 72 | 0.15 | 0.32 | 0.13 | 0.31 | 0.31 | 72 |
| 88 | 0.03 | 0.09 | 0.03 | 0.16 | 0.12 | 84 |
| 89 | 0.06 | 0.19 | 0.06 | 0.33 | 0.26 | 13 |
| 93 | 0.63 | 1.33 | 0.64 | 1.50 | 1.42 | 88 |
| 94 | 0.87 | 1.77 | 0.89 | 2.27 | 2.02 | 89 |
| 95 | 0.12 | 0.26 | 0.14 | 0.33 | 0.30 | 90 |
| 96 | 0.16 | 0.33 | 0.14 | 0.32 | 0.32 | 91 |
| 97 | 0.35 | 0.75 | 0.23 | 0.55 | 0.65 | 92 |
| 98 | 0.67 | 1.36 | 0.71 | 1.82 | 1.59 | 93 |
| 99 | 0.65 | 1.31 | 0.63 | 1.62 | 1.46 | 94 |
| 105 | 0.09 | 0.19 | 0.09 | 0.23 | 0.21 | 100 |
| 108 | 0.45 | 0.92 | 0.43 | 1.09 | 1.00 | 103 |
| 109 | 0.07 | 0.24 | 0.06 | 0.35 | 0.29 | 104 |
| 110 | 0.10 | 0.34 | 0.09 | 0.51 | 0.43 | 105 |
| 120 | 1.00 | 2.05 | 0.89 | 2.26 | 2.16 | 115 |
| 121 | 0.22 | 0.45 | 0.21 | 0.53 | 0.49 | 116 |
| 127 | 0.44 | 0.89 | 0.41 | 1.06 | 0.97 | 120 |
| 128 | 0.35 | 0.83 | 0.39 | 0.93 | 0.88 | 121 |
| 129 | 0.30 | 0.71 | 0.34 | 0.80 | 0.75 | 122 |
| 130 | 0.53 | 1.28 | 0.56 | 1.33 | 1.31 | 123 |
| 131 | 0.89 | 2.14 | 0.76 | 1.81 | 1.98 | 124 |
| 132 | 0.42 | 1.00 | 0.48 | 1.13 | 1.07 | 125 |
| 133 | 0.23 | 0.56 | 0.24 | 0.57 | 0.56 | 126 |
| 134 | 0.43 | 1.04 | 0.43 | 1.02 | 1.03 | 127 |
| 139 | 0.13 | 0.31 | 0.10 | 0.24 | 0.27 | 132 |
| 144 | 0.23 | 0.56 | 0.06 | 0.14 | 0.35 | 137 |
| 145 | 0.65 | 1.55 | 0.56 | 1.34 | 1.44 | 138 |
| 150 | 0.14 | 0.35 | 0.05 | 0.12 | 0.24 | 143 |
| 152 | 0.23 | 0.56 | 0.23 | 0.53 | 0.55 | 145 |
| 153 | 0.17 | 0.41 | 0.23 | 0.54 | 0.48 | 146 |
| 154 | 0.51 | 1.35 | 0.51 | 1.35 | 1.35 | 147 |
| 155 | 0.24 | 0.65 | 0.22 | 0.59 | 0.62 | 148 |
| 156 | 0.36 | 0.96 | 0.41 | 1.09 | 1.03 | 149 |
| 157 | 0.58 | 1.55 | 0.51 | 1.35 | 1.45 | 150 |
| 158 | 0.22 | 0.59 | 0.20 | 0.52 | 0.56 | 151 |
| 159 | 0.32 | 0.86 | 0.27 | 0.72 | 0.79 | 152 |
| 160 | 0.23 | 0.61 | 0.21 | 0.54 | 0.58 | 153 |
| 161 | 0.20 | 0.53 | 0.20 | 0.53 | 0.53 | 154 |
| 162 | 0.45 | 1.19 | 0.44 | 1.17 | 1.18 | 155 |
| 163 | 0.38 | 1.00 | 0.33 | 0.87 | 0.93 | 156 |
| 164 | 0.24 | 0.63 | 0.21 | 0.55 | 0.59 | 157 |
| 165 | 0.58 | 1.53 | 0.59 | 1.57 | 1.55 | 158 |
| 166 | 0.12 | 0.31 | 0.11 | 0.28 | 0.29 | 159 |
| 168 | 0.15 | 0.39 | 0.14 | 0.37 | 0.38 | 161 |

TABLE 9
Ratio of ATN-1 gene expression level in A204
cell administered with gapmer (1.0 μM) having
2′-O—MOE group in wing nucleoside

| | | Ratio of | | Ratio of | Average | |
|---|---|---|---|---|---|---|
| | | mRNA | | mRNA | of | |
| | mRNA | expression | mRNA | expression | ratio of | |
| | expression | level/ | expression | level/ | mRNA | SEQ |
| ASO | level | NRH-71 | level | NRH-71 | expression | ID |
| No. | (exon4-5) | (exon4-5) | (exon6-7) | (exon6-7) | level | NO: |
| 69 | 0.16 | 0.23 | 0.16 | 0.24 | 0.23 | 69 |
| 76 | 0.27 | 0.39 | 0.25 | 0.37 | 0.38 | 76 |
| 77 | 0.23 | 0.34 | 0.18 | 0.27 | 0.31 | 77 |
| 78 | 0.26 | 0.39 | 0.24 | 0.36 | 0.37 | 10 |
| 79 | 0.26 | 0.39 | 0.27 | 0.40 | 0.39 | 78 |
| 80 | 0.64 | 0.92 | 0.70 | 1.01 | 0.97 | 79 |
| 81 | 0.66 | 0.95 | 0.48 | 0.69 | 0.82 | 80 |
| 82 | 0.93 | 1.35 | 1.00 | 1.54 | 1.44 | 81 |
| 83 | 0.82 | 1.19 | 0.79 | 1.15 | 1.17 | 82 |
| 84 | 0.44 | 0.64 | 0.46 | 0.67 | 0.66 | 11 |
| 85 | 0.26 | 0.38 | 0.27 | 0.40 | 0.39 | 83 |
| 86 | 0.16 | 0.23 | 0.17 | 0.25 | 0.24 | 12 |
| 88 | 0.07 | 0.10 | 0.08 | 0.11 | 0.11 | 84 |
| 89 | 0.21 | 0.32 | 0.21 | 0.33 | 0.33 | 13 |
| 90 | 0.27 | 0.39 | 0.28 | 0.42 | 0.41 | 85 |
| 109 | 0.31 | 0.44 | 0.28 | 0.43 | 0.44 | 104 |
| 110 | 0.33 | 0.48 | 0.35 | 0.54 | 0.51 | 105 |
| 121 | 0.44 | 0.65 | 0.39 | 0.63 | 0.64 | 116 |
| 139 | 0.31 | 0.46 | 0.26 | 0.41 | 0.43 | 132 |
| 144 | 0.41 | 0.61 | 0.09 | 0.14 | 0.38 | 137 |
| 166 | 0.20 | 0.31 | 0.19 | 0.33 | 0.32 | 159 |
| 168 | 0.30 | 0.46 | 0.26 | 0.45 | 0.45 | 161 |

TABLE 10
Ratio of ATN-1 gene expression level in A204
cell administered with gapmer (0.5 μM) having
2′-O—MOE group in wing nucleoside

| | | Ratio of | | Ratio of | Average | |
|---|---|---|---|---|---|---|
| | | mRNA | | mRNA | of | |
| | mRNA | expression | mRNA | expression | ratio of | |
| | expression | level/ | expression | level/ | mRNA | SEQ |
| ASO | level | NRH-71 | level | NRH-71 | expression | ID |
| No. | (exon4-5) | (exon4-5) | (exon6-7) | (exon6-7) | level | NO: |
| 62 | 0.24 | 0.31 | 0.39 | 0.52 | 0.41 | 62 |
| 63 | 0.30 | 0.38 | 0.42 | 0.56 | 0.47 | 63 |
| 66 | 0.88 | 1.12 | 0.72 | 0.94 | 1.03 | 66 |
| 67 | 0.79 | 1.00 | 0.72 | 0.96 | 0.98 | 67 |
| 68 | 0.16 | 0.19 | 0.20 | 0.23 | 0.21 | 68 |
| 70 | 0.26 | 0.31 | 0.29 | 0.33 | 0.32 | 70 |
| 73 | 0.72 | 0.87 | 0.77 | 0.89 | 0.88 | 73 |
| 74 | 0.83 | 1.00 | 0.87 | 1.00 | 1.00 | 74 |
| 75 | 0.82 | 0.99 | 0.87 | 0.99 | 0.99 | 75 |
| 91 | 0.93 | 1.18 | 0.96 | 1.34 | 1.26 | 86 |
| 92 | 0.79 | 1.01 | 0.84 | 1.16 | 1.09 | 87 |
| 100 | 0.50 | 0.60 | 0.58 | 0.67 | 0.63 | 95 |
| 101 | 0.72 | 0.87 | 0.69 | 0.79 | 0.83 | 96 |
| 102 | 0.55 | 0.66 | 0.55 | 0.63 | 0.64 | 97 |
| 103 | 0.41 | 0.50 | 0.42 | 0.48 | 0.49 | 98 |
| 104 | 0.73 | 0.88 | 0.80 | 0.92 | 0.90 | 99 |
| 106 | 0.28 | 0.33 | 0.33 | 0.37 | 0.35 | 101 |
| 107 | 0.72 | 0.87 | 0.72 | 0.82 | 0.85 | 102 |
| 111 | 0.52 | 0.66 | 0.51 | 0.72 | 0.69 | 106 |
| 112 | 0.46 | 0.58 | 0.47 | 0.66 | 0.62 | 107 |
| 113 | 0.89 | 1.10 | 0.84 | 0.95 | 1.03 | 108 |
| 114 | 0.94 | 1.16 | 0.86 | 0.97 | 1.07 | 109 |
| 115 | 0.73 | 0.91 | 0.73 | 0.83 | 0.87 | 110 |
| 116 | 0.76 | 0.94 | 0.68 | 0.77 | 0.86 | 111 |
| 117 | 0.11 | 0.14 | 0.11 | 0.13 | 0.13 | 112 |
| 118 | 0.18 | 0.22 | 0.20 | 0.23 | 0.22 | 113 |
| 119 | 0.35 | 0.43 | 0.37 | 0.42 | 0.43 | 114 |
| 122 | 0.67 | 0.80 | 0.66 | 0.90 | 0.85 | 117 |
| 124 | 0.31 | 0.37 | 0.29 | 0.39 | 0.38 | 30 |
| 125 | 0.97 | 1.17 | 0.80 | 1.09 | 1.13 | 118 |
| 135 | 0.56 | 0.69 | 0.51 | 0.58 | 0.64 | 128 |
| 136 | 0.79 | 0.98 | 0.74 | 0.83 | 0.91 | 129 |
| 137 | 0.47 | 0.58 | 0.47 | 0.53 | 0.56 | 130 |
| 138 | 0.38 | 0.46 | 0.38 | 0.43 | 0.45 | 131 |
| 140 | 0.25 | 0.30 | 0.22 | 0.25 | 0.28 | 133 |
| 141 | 0.45 | 0.56 | 0.47 | 0.54 | 0.55 | 134 |
| 142 | 0.92 | 1.14 | 0.86 | 0.97 | 1.05 | 135 |
| 143 | 0.65 | 0.84 | 0.67 | 0.79 | 0.82 | 136 |
| 146 | 0.96 | 1.16 | 0.95 | 1.09 | 1.12 | 139 |
| 147 | 0.64 | 0.77 | 0.72 | 0.83 | 0.80 | 140 |
| 148 | 0.85 | 1.02 | 0.85 | 0.98 | 1.00 | 141 |
| 149 | 0.90 | 1.13 | 0.91 | 1.12 | 1.13 | 142 |
| 151 | 0.80 | 0.97 | 0.82 | 0.95 | 0.96 | 144 |
| 161 | 0.50 | 0.72 | 0.59 | 0.73 | 0.73 | 154 |
| 167 | 0.25 | 0.32 | 0.25 | 0.30 | 0.31 | 160 |
| 169 | 0.49 | 0.63 | 0.51 | 0.60 | 0.61 | 162 |
| 170 | 0.39 | 0.50 | 0.42 | 0.50 | 0.50 | 163 |

TABLE 11
Ratio of ATN-1 gene expression level in A204
cell administered with gapmer (0.5 μM) having
2′-O—MOE group in wing nucleoside

| | | Ratio of | | Ratio of | Average | |
|---|---|---|---|---|---|---|
| | | mRNA | | mRNA | of | |
| | mRNA | expression | mRNA | expression | ratio of | |
| | expression | level/ | expression | level/ | mRNA | SEQ |
| ASO | level | NRH-71 | level | NRH-71 | expression | ID |
| No. | (exon4-5) | (exon4-5) | (exon6-7) | (exon6-7) | level | NO: |
| 171 | 0.63 | 0.89 | 0.68 | 0.83 | 0.86 | 164 |
| 172 | 0.73 | 1.03 | 0.73 | 0.89 | 0.96 | 165 |
| 173 | 0.68 | 0.96 | 0.64 | 0.78 | 0.87 | 166 |
| 174 | 0.61 | 0.72 | 0.65 | 0.79 | 0.75 | 167 |
| 175 | 0.50 | 0.59 | 0.48 | 0.58 | 0.58 | 168 |
| 176 | 0.52 | 0.58 | 0.57 | 0.64 | 0.61 | 169 |
| 177 | 0.32 | 0.39 | 0.35 | 0.39 | 0.39 | 170 |
| 178 | 0.57 | 0.78 | 0.61 | 0.71 | 0.74 | 171 |
| 179 | 1.01 | 1.42 | 1.10 | 1.35 | 1.39 | 172 |
| 180 | 0.57 | 0.81 | 0.68 | 0.83 | 0.82 | 173 |
| 181 | 0.86 | 0.87 | 0.85 | 1.00 | 0.93 | 21 |
| 182 | 0.73 | 0.74 | 0.67 | 0.78 | 0.76 | 174 |
| 183 | 0.60 | 0.62 | 0.57 | 0.63 | 0.63 | 175 |
| 184 | 0.50 | 0.52 | 0.53 | 0.59 | 0.56 | 176 |
| 185 | 0.31 | 0.35 | 0.33 | 0.38 | 0.37 | 177 |
| 186 | 0.93 | 0.95 | 0.92 | 1.07 | 1.01 | 178 |
| 187 | 0.82 | 0.83 | 0.75 | 0.88 | 0.86 | 179 |
| 188 | 0.63 | 0.64 | 0.59 | 0.69 | 0.67 | 180 |
| 189 | 0.52 | 0.54 | 0.58 | 0.64 | 0.59 | 181 |
| 190 | 0.48 | 0.46 | 0.57 | 0.55 | 0.51 | 182 |
| 191 | 0.96 | 0.97 | 0.88 | 1.03 | 1.00 | 183 |
| 192 | 0.79 | 0.80 | 0.76 | 0.89 | 0.84 | 184 |
| 193 | 0.77 | 1.09 | 0.84 | 1.03 | 1.06 | 185 |
| 194 | 0.81 | 0.96 | 0.88 | 1.05 | 1.01 | 186 |
| 195 | 0.73 | 0.87 | 0.81 | 0.97 | 0.92 | 187 |
| 196 | 0.79 | 0.76 | 0.81 | 0.79 | 0.78 | 188 |
| 197 | 0.86 | 0.86 | 0.84 | 0.87 | 0.87 | 189 |
| 198 | 0.60 | 0.59 | 0.71 | 0.69 | 0.64 | 190 |
| 199 | 1.03 | 1.03 | 0.92 | 0.95 | 0.99 | 191 |
| 200 | 0.82 | 0.83 | 0.80 | 0.94 | 0.88 | 192 |
| 201 | 0.55 | 0.56 | 0.56 | 0.66 | 0.61 | 193 |
| 202 | 0.60 | 0.62 | 0.63 | 0.69 | 0.66 | 194 |
| 203 | 0.62 | 0.60 | 0.69 | 0.67 | 0.64 | 195 |
| 204 | 0.31 | 0.34 | 0.32 | 0.34 | 0.34 | 196 |
| 205 | 0.45 | 0.51 | 0.46 | 0.52 | 0.52 | 197 |
| 206 | 0.65 | 0.74 | 0.67 | 0.76 | 0.75 | 198 |
| 207 | 0.36 | 0.41 | 0.41 | 0.42 | 0.41 | 199 |
| 208 | 0.55 | 0.57 | 0.62 | 0.68 | 0.63 | 200 |
| 209 | 0.53 | 0.55 | 0.63 | 0.69 | 0.62 | 201 |
| 210 | 0.67 | 0.65 | 0.75 | 0.73 | 0.69 | 202 |
| 211 | 0.91 | 0.88 | 0.91 | 0.88 | 0.88 | 203 |
| 212 | 1.00 | 0.97 | 0.92 | 0.90 | 0.93 | 204 |
| 213 | 0.80 | 0.90 | 0.74 | 0.77 | 0.83 | 205 |
| 214 | 0.76 | 0.84 | 0.78 | 0.80 | 0.82 | 206 |
| 215 | 0.81 | 0.78 | 0.81 | 0.79 | 0.79 | 207 |
| 216 | 0.90 | 0.87 | 0.77 | 0.75 | 0.81 | 208 |
| 217 | 0.67 | 0.95 | 0.78 | 0.96 | 0.95 | 209 |
| 218 | 0.65 | 0.92 | 0.79 | 0.96 | 0.94 | 210 |
| 219 | 0.62 | 0.87 | 0.68 | 0.83 | 0.85 | 211 |
| 220 | 0.69 | 0.86 | 0.70 | 0.80 | 0.83 | 212 |
| 221 | 0.75 | 0.89 | 0.85 | 1.02 | 0.95 | 213 |
| 222 | 0.79 | 0.93 | 0.81 | 0.97 | 0.95 | 214 |
| 223 | 0.82 | 0.97 | 0.88 | 1.06 | 1.02 | 215 |
| 224 | 0.67 | 0.95 | 0.74 | 0.90 | 0.93 | 216 |
| 225 | 0.80 | 1.13 | 0.88 | 1.08 | 1.11 | 217 |
| 226 | 0.86 | 1.21 | 0.96 | 1.18 | 1.19 | 218 |
| 227 | 0.48 | 0.56 | 0.49 | 0.54 | 0.55 | 219 |
| 228 | 0.51 | 0.63 | 0.53 | 0.61 | 0.62 | 220 |
| 229 | 0.39 | 0.47 | 0.39 | 0.45 | 0.46 | 221 |
| 230 | 0.81 | 0.96 | 0.85 | 1.02 | 0.99 | 222 |
| 241 | 0.69 | 0.67 | 0.69 | 0.67 | 0.67 | 233 |
| 242 | 0.80 | 0.77 | 0.90 | 0.87 | 0.82 | 234 |
| 358 | 0.72 | 0.81 | 0.72 | 0.74 | 0.78 | 350 |
| 359 | 0.73 | 0.71 | 0.67 | 0.65 | 0.68 | 351 |
| 360 | 0.75 | 0.72 | 0.71 | 0.69 | 0.71 | 352 |
| 361 | 0.73 | 0.71 | 0.69 | 0.67 | 0.69 | 353 |
| 362 | 0.82 | 0.80 | 0.80 | 0.78 | 0.79 | 354 |
| 363 | 0.88 | 0.85 | 0.90 | 0.88 | 0.86 | 355 |
| 473 | 0.45 | 0.58 | 0.45 | 0.58 | 0.58 | 472 |
| 474 | 0.48 | 0.62 | 0.41 | 0.54 | 0.58 | 472 |
| 475 | 0.55 | 0.71 | 0.44 | 0.58 | 0.64 | 472 |
| 476 | 0.43 | 0.56 | 0.47 | 0.61 | 0.59 | 472 |
| 477 | 0.64 | 0.83 | 0.61 | 0.80 | 0.82 | 472 |
| 478 | 0.58 | 0.74 | 0.51 | 0.66 | 0.70 | 472 |
| 479 | 0.57 | 0.74 | 0.48 | 0.62 | 0.68 | 472 |
| 480 | 0.09 | 0.11 | 0.10 | 0.13 | 0.12 | 177 |
| 481 | 0.10 | 0.13 | 0.14 | 0.18 | 0.16 | 177 |
| 482 | 0.13 | 0.17 | 0.13 | 0.17 | 0.17 | 177 |
| 483 | 0.07 | 0.09 | 0.07 | 0.09 | 0.09 | 177 |
| 484 | 0.19 | 0.25 | 0.17 | 0.23 | 0.24 | 177 |
| 485 | 0.16 | 0.20 | 0.13 | 0.16 | 0.18 | 177 |
| 486 | 0.10 | 0.13 | 0.08 | 0.10 | 0.12 | 177 |
| 487 | 0.44 | 0.44 | 0.41 | 0.42 | 0.43 | 133 |
| 488 | 0.26 | 0.26 | 0.28 | 0.29 | 0.28 | 133 |
| 489 | 0.39 | 0.39 | 0.44 | 0.45 | 0.42 | 133 |
| 490 | 0.32 | 0.32 | 0.24 | 0.24 | 0.28 | 133 |
| 491 | 0.43 | 0.43 | 0.43 | 0.45 | 0.44 | 133 |
| 492 | 0.36 | 0.36 | 0.40 | 0.41 | 0.39 | 133 |
| 493 | 0.37 | 0.37 | 0.36 | 0.37 | 0.37 | 133 |
| 494 | 0.13 | 0.14 | 0.10 | 0.11 | 0.13 | 196 |
| 495 | 0.14 | 0.16 | 0.11 | 0.12 | 0.14 | 196 |
| 497 | 0.29 | 0.31 | 0.30 | 0.32 | 0.31 | 473 |
| 498 | 0.15 | 0.16 | 0.13 | 0.14 | 0.15 | 473 |
| 499 | 0.20 | 0.21 | 0.18 | 0.19 | 0.20 | 473 |

1. An antisense oligonucleotide consisting of 15 to 22 nucleotides complementary to a nucleic acid comprising at least 15 consecutive bases in a region selected from the group consisting of positions 1 to 84, 142 to 168, 197 to 224, 286 to 317, 324 to 370, 391 to 434, 519 to 540, 623 to 643, 690 to 780, 824 to 855, 860 to 897, 948 to 987, 1044 to 1072, 1125 to 1174, 1181 to 1213, 1228 to 1255, 1265 to 1327, 1334 to 1356, 1416 to 1440, 1447 to 1631, 1638 to 1667, 1675 to 1705, 1748 to 1823, 1838 to 1861, 1870 to 1913, 1920 to 1941, 2000 to 2040, 2047 to 2075, 2086 to 2120, 2129 to 2187, 2194 to 2415, 2451 to 2497, 2592 to 2759, 2766 to 2870, 2928 to 2948, 2955 to 2989, 3021 to 3086, 3133 to 3209, 3217 to 3284, 3295 to 3350, 3384 to 3436, 3562 to 3771, 3858 to 3905, 3931 to 4039, 4067 to 4134, 4170 to 4232, 4241 to 4283, and 4286 to 4355 of a base sequence of SEQ ID NO: 471, or a pharmaceutically acceptable salt thereof, or a hydrate thereof.

2. The antisense oligonucleotide or a pharmaceutically acceptable salt thereof, or a hydrate thereof according to claim 1, wherein the antisense oligonucleotide is complementary to a nucleic acid comprising at least 15 consecutive bases in a target region selected from the group consisting of positions 1 to 69, 331 to 363, 398 to 427, 697 to 773, 867 to 890, 955 to 980, 1132 to 1167, 1272 to 1320, 1454 to 1624, 1682 to 1705, 1748 to 1816, 1877 to 1906, 2007 to 2033, 2093 to 2113, 2136 to 2180, 2201 to 2408, 2458 to 2490, 2599 to 2752, 2773 to 2863, 2962 to 2982, 3028 to 3079, 3140 to 3202, 3224 to 3277, 3302 to 3343, 3391 to 3429, 3569 to 3764, 3865 to 3898, 3938 to 4032, 4074 to 4127, 4177 to 4225, 4248 to 4276, and 4293 to 4355 of the base sequence of SEQ ID NO: 471.

3. The antisense oligonucleotide or a pharmaceutically acceptable salt thereof, or a hydrate thereof according to claim 1, wherein the antisense oligonucleotide is complementary to a nucleic acid comprising at least 15 consecutive bases in a target region selected from the group consisting of positions 14 to 33, 34 to 53, 54 to 73, 148 to 167, 397 to 432, 723 to 742, 747 to 778, 965 to 984, 1127 to 1146, 1148 to 1173, 1182 to 1213, 1228 to 1254, 1270 to 1308, 1484 to 1564, 1578 to 1629, 1686 to 1705, 1748 to 1767, 1768 to 1787, 1794 to 1813, 1870 to 1913, 2015 to 2034, 2086 to 2118, 2129 to 2148, 2151 to 2184, 2206 to 2225, 2239 to 2272, 2287 to 2336, 2337 to 2373, 2387 to 2406, 2598 to 2757, 2775 to 2809, 2826 to 2866, 2959 to 2982, 3032 to 3051, 3052 to 3071, 3136 to 3155, 3168 to 3187, 3188 to 3207, 3232 to 3270, 3298 to 3344, 3408 to 3427, 3563 to 3582, 3590 to 3728, 3739 to 3769, 3877 to 3896, 3949 to 4030, and 4170 to 4225 of the base sequence of SEQ ID NO: 471.

4. The antisense oligonucleotide or a pharmaceutically acceptable salt thereof, or a hydrate thereof according to claim 3, wherein the antisense oligonucleotide is complementary to a nucleic acid comprising at least 15 consecutive bases in a target region selected from the group consisting of positions 14 to 33, 34 to 53, 54 to 73, 148 to 167, 397 to 416, 398 to 417, 399 to 418, 400 to 419, 401 to 420, 402 to 421, 403 to 422, 404 to 423, 405 to 424, 406 to 425, 407 to 426, 408 to 427, 413 to 432, 723 to 742, 747 to 766, 756 to 775, 759 to 778, 965 to 984, 1127 to 1146, 1148 to 1167, 1151 to 1170, 1154 to 1173, 1182 to 1201, 1185 to 1204, 1188 to 1207, 1191 to 1210, 1194 to 1213, 1228 to 1247, 1231 to 1250, 1235 to 1254, 1270 to 1289, 1289 to 1308, 1484 to 1503, 1488 to 1507, 1491 to 1510, 1493 to 1512, 1506 to 1525, 1517 to 1536, 1531 to 1550, 1545 to 1564, 1578 to 1597, 1579 to 1598, 1580 to 1599, 1581 to 1600, 1582 to 1601, 1583 to 1602, 1584 to 1603, 1585 to 1604, 1586 to 1605, 1587 to 1606, 1588 to 1607, 1589 to 1608, 1590 to 1609, 1591 to 1610, 1592 to 1611, 1593 to 1612, 1594 to 1613, 1595 to 1614, 1596 to 1615, 1597 to 1616, 1598 to 1617, 1599 to 1618, 1600 to 1619, 1601 to 1620, 1602 to 1621, 1603 to 1622, 1604 to 1623, 1605 to 1624, 1607 to 1626, 1610 to 1629, 1686 to 1705, 1748 to 1767, 1768 to 1787, 1794 to 1813, 1870 to 1889, 1871 to 1890, 1874 to 1893, 1877 to 1896, 1878 to 1897, 1879 to 1898, 1880 to 1899, 1881 to 1900, 1882 to 1901, 1883 to 1902, 1884 to 1903, 1885 to 1904, 1886 to 1905, 1887 to 1906, 1889 to 1908, 1894 to 1913, 2015 to 2034, 2086 to 2105, 2093 to 2112, 2094 to 2113, 2097 to 2116, 2099 to 2118, 2129 to 2148, 2151 to 2170, 2165 to 2184, 2206 to 2225, 2239 to 2258, 2253 to 2272, 2287 to 2306, 2301 to 2320, 2317 to 2336, 2337 to 2356, 2354 to 2373, 2387 to 2406, 2598 to 2617, 2613 to 2632, 2614 to 2633, 2615 to 2634, 2616 to 2635, 2617 to 2636, 2618 to 2637, 2619 to 2638, 2620 to 2639, 2621 to 2640, 2622 to 2641, 2623 to 2642, 2624 to 2643, 2625 to 2644, 2626 to 2645, 2627 to 2646, 2628 to 2647, 2629 to 2648, 2630 to 2649, 2631 to 2650, 2632 to 2651, 2633 to 2652, 2634 to 2653, 2635 to 2654, 2636 to 2655, 2637 to 2656, 2638 to 2657, 2639 to 2658, 2640 to 2659, 2641 to 2660, 2642 to 2661, 2643 to 2662, 2644 to 2663, 2645 to 2664, 2646 to 2665, 2647 to 2666, 2648 to 2667, 2649 to 2668, 2663 to 2682, 2672 to 2691, 2673 to 2692, 2674 to 2693, 2675 to 2694, 2676 to 2695, 2677 to 2696, 2678 to 2697, 2679 to 2698, 2680 to 2699, 2681 to 2700, 2682 to 2701, 2683 to 2702, 2684 to 2703, 2685 to 2704, 2686 to 2705, 2687 to 2706, 2688 to 2707, 2689 to 2708, 2690 to 2709, 2691 to 2710, 2692 to 2711, 2693 to 2712, 2694 to 2713, 2695 to 2714, 2696 to 2715, 2697 to 2716, 2698 to 2717, 2699 to 2718, 2700 to 2719, 2701 to 2720, 2702 to 2721, 2703 to 2722, 2704 to 2723, 2705 to 2724, 2706 to 2725, 2707 to 2726, 2708 to 2727, 2709 to 2728, 2710 to 2729, 2711 to 2730, 2712 to 2731, 2713 to 2732, 2714 to 2733, 2715 to 2734, 2716 to 2735, 2717 to 2736, 2718 to 2737, 2719 to 2738, 2720 to 2739, 2721 to 2740, 2722 to 2741, 2723 to 2742, 2724 to 2743, 2725 to 2744, 2726 to 2745, 2727 to 2746, 2728 to 2747, 2729 to 2748, 2730 to 2749, 2731 to 2750, 2732 to 2751, 2733 to 2752, 2738 to 2757, 2775 to 2794, 2790 to 2809, 2826 to 2845, 2827 to 2846, 2828 to 2847, 2829 to 2848, 2830 to 2849, 2831 to 2850, 2832 to 2851, 2833 to 2852, 2834 to 2853, 2835 to 2854, 2836 to 2855, 2837 to 2856, 2838 to 2857, 2839 to 2858, 2840 to 2859, 2841 to 2860, 2842 to 2861, 2843 to 2862, 2844 to 2863, 2847 to 2866, 2959 to 2978, 2962 to 2981, 2963 to 2982, 3032 to 3051, 3052 to 3071, 3136 to 3155, 3168 to 3187, 3188 to 3207, 3232 to 3251, 3251 to 3270, 3298 to 3317, 3302 to 3321, 3303 to 3322, 3304 to 3323, 3305 to 3324, 3306 to 3325, 3307 to 3326, 3308 to 3327, 3309 to 3328, 3310 to 3329, 3311 to 3330, 3312 to 3331, 3313 to 3332, 3314 to 3333, 3315 to 3334, 3316 to 3335, 3317 to 3336, 3318 to 3337, 3319 to 3338, 3320 to 3339, 3321 to 3340, 3322 to 3341, 3323 to 3342, 3324 to 3343, 3325 to 3344, 3408 to 3427, 3563 to 3582, 3590 to 3609, 3608 to 3627, 3617 to 3636, 3618 to 3637, 3619 to 3638, 3620 to 3639, 3621 to 3640, 3622 to 3641, 3623 to 3642, 3624 to 3643, 3625 to 3644, 3626 to 3645, 3627 to 3646, 3628 to 3647, 3629 to 3648, 3630 to 3649, 3631 to 3650, 3632 to 3651, 3633 to 3652, 3634 to 3653, 3635 to 3654, 3636 to 3655, 3637 to 3656, 3638 to 3657, 3639 to 3658, 3640 to 3659, 3641 to 3660, 3642 to 3661, 3643 to 3662, 3644 to 3663, 3645 to 3664, 3646 to 3665, 3647 to 3666, 3648 to 3667, 3649 to 3668, 3650 to 3669, 3651 to 3670, 3652 to 3671, 3653 to 3672, 3654 to 3673, 3671 to 3690, 3672 to 3691, 3673 to 3692, 3674 to 3693, 3675 to 3694, 3676 to 3695, 3677 to 3696, 3678 to 3697, 3679 to 3698, 3680 to 3699, 3681 to 3700, 3682 to 3701, 3683 to 3702, 3684 to 3703, 3685 to 3704, 3686 to 3705, 3687 to 3706, 3688 to 3707, 3689 to 3708, 3690 to 3709, 3691 to 3710, 3692 to 3711, 3693 to 3712, 3694 to 3713, 3695 to 3714, 3696 to 3715, 3697 to 3716, 3698 to 3717, 3699 to 3718, 3700 to 3719, 3701 to 3720, 3702 to 3721, 3703 to 3722, 3704 to 3723, 3705 to 3724, 3706 to 3725, 3707 to 3726, 3708 to 3727, 3709 to 3728, 3739 to 3758, 3740 to 3759, 3741 to 3760, 3742 to 3761, 3743 to 3762, 3744 to 3763, 3745 to 3764, 3750 to 3769, 3877 to 3896, 3949 to 3968, 3964 to 3983, 3965 to 3984, 3966 to 3985, 3967 to 3986, 3968 to 3987, 3969 to 3988, 3970 to 3989, 3971 to 3990, 3972 to 3991, 3973 to 3992, 3974 to 3993, 3975 to 3994, 3976 to 3995, 3977 to 3996, 3978 to 3997, 3979 to 3998, 3980 to 3999, 3981 to 4000, 3982 to 4001, 3983 to 4002, 3984 to 4003, 3985 to 4004, 3986 to 4005, 3987 to 4006, 3988 to 4007, 3989 to 4008, 3990 to 4009, 3991 to 4010, 3992 to 4011, 3993 to 4012, 3994 to 4013, 3995 to 4014, 3996 to 4015, 3997 to 4016, 3998 to 4017, 3999 to 4018, 4011 to 4030, 4170 to 4189, 4173 to 4192, 4176 to 4195, 4177 to 4196, 4178 to 4197, 4179 to 4198, 4180 to 4199, 4181 to 4200, 4182 to 4201, 4183 to 4202, 4184 to 4203, 4185 to 4204, 4186 to 4205, 4187 to 4206, 4188 to 4207, 4189 to 4208, 4190 to 4209, 4191 to 4210, 4192 to 4211, 4193 to 4212, 4194 to 4213, 4195 to 4214, 4196 to 4215, 4197 to 4216, 4198 to 4217, 4199 to 4218, 4200 to 4219, 4201 to 4220, 4202 to 4221, 4203 to 4222, 4204 to 4223, 4205 to 4224, and 4206 to 4225 of the base sequence of SEQ ID NO: 471.

5. The antisense oligonucleotide or a pharmaceutically acceptable salt thereof, or a hydrate thereof according to claim 2 or 3, wherein the antisense oligonucleotide is complementary to a nucleic acid comprising at least 15 consecutive bases in a target region selected from the group consisting of positions 14 to 33, 34 to 53, 398 to 427, 723 to 742, 747 to 766, 1148 to 1167, 1289 to 1308, 1484 to 1564, 1578 to 1624, 1686 to 1705, 1748 to 1767, 1768 to 1787, 1794 to 1813, 1877 to 1906, 2093 to 2113, 2151 to 2170, 2206 to 2225, 2239 to 2272, 2287 to 2336, 2337 to 2373, 2387 to 2406, 2613 to 2752, 2775 to 2809, 2826 to 2863, 2962 to 2982, 3032 to 3051, 3052 to 3071, 3168 to 3187, 3232 to 3270, 3302 to 3343, 3408 to 3427, 3590 to 3728, 3739 to 3764, 3877 to 3896, 3949 to 4030, and 4177 to 4225 of the base sequence of SEQ ID NO: 471.

6. The antisense oligonucleotide or a pharmaceutically acceptable salt thereof, or a hydrate thereof according to claim 5, wherein the antisense oligonucleotide is complementary to a nucleic acid comprising at least 15 consecutive bases in a target region selected from the group consisting of positions 14 to 33, 34 to 53, 398 to 417, 399 to 418, 400 to 419, 401 to 420, 402 to 421, 403 to 422, 404 to 423, 405 to 424, 406 to 425, 407 to 426, 408 to 427, 723 to 742, 747 to 766, 1148 to 1167, 1289 to 1308, 1484 to 1503, 1488 to 1507, 1491 to 1510, 1493 to 1512, 1506 to 1525, 1517 to 1536, 1531 to 1550, 1545 to 1564, 1578 to 1597, 1579 to 1598, 1580 to 1599, 1581 to 1600, 1582 to 1601, 1583 to 1602, 1584 to 1603, 1585 to 1604, 1586 to 1605, 1587 to 1606, 1588 to 1607, 1589 to 1608, 1590 to 1609, 1591 to 1610, 1592 to 1611, 1593 to 1612, 1594 to 1613, 1595 to 1614, 1596 to 1615, 1597 to 1616, 1598 to 1617, 1599 to 1618, 1600 to 1619, 1601 to 1620, 1602 to 1621, 1603 to 1622, 1604 to 1623, 1605 to 1624, 1686 to 1705, 1748 to 1767, 1768 to 1787, 1794 to 1813, 1877 to 1896, 1878 to 1897, 1879 to 1898, 1880 to 1899, 1881 to 1900, 1882 to 1901, 1883 to 1902, 1884 to 1903, 1885 to 1904, 1886 to 1905, 1887 to 1906, 2093 to 2112, 2094 to 2113, 2151 to 2170, 2206 to 2225, 2239 to 2258, 2253 to 2272, 2287 to 2306, 2301 to 2320, 2317 to 2336, 2337 to 2356, 2354 to 2373, 2387 to 2406, 2613 to 2632, 2614 to 2633, 2615 to 2634, 2616 to 2635, 2617 to 2636, 2618 to 2637, 2619 to 2638, 2620 to 2639, 2621 to 2640, 2622 to 2641, 2623 to 2642, 2624 to 2643, 2625 to 2644, 2626 to 2645, 2627 to 2646, 2628 to 2647, 2629 to 2648, 2630 to 2649, 2631 to 2650, 2632 to 2651, 2633 to 2652, 2634 to 2653, 2635 to 2654, 2636 to 2655, 2637 to 2656, 2638 to 2657, 2639 to 2658, 2640 to 2659, 2641 to 2660, 2642 to 2661, 2643 to 2662, 2644 to 2663, 2645 to 2664, 2646 to 2665, 2647 to 2666, 2648 to 2667, 2649 to 2668, 2663 to 2682, 2672 to 2691, 2673 to 2692, 2674 to 2693, 2675 to 2694, 2676 to 2695, 2677 to 2696, 2678 to 2697, 2679 to 2698, 2680 to 2699, 2681 to 2700, 2682 to 2701, 2683 to 2702, 2684 to 2703, 2685 to 2704, 2686 to 2705, 2687 to 2706, 2688 to 2707, 2689 to 2708, 2690 to 2709, 2691 to 2710, 2692 to 2711, 2693 to 2712, 2694 to 2713, 2695 to 2714, 2696 to 2715, 2697 to 2716, 2698 to 2717, 2699 to 2718, 2700 to 2719, 2701 to 2720, 2702 to 2721, 2703 to 2722, 2704 to 2723, 2705 to 2724, 2706 to 2725, 2707 to 2726, 2708 to 2727, 2709 to 2728, 2710 to 2729, 2711 to 2730, 2712 to 2731, 2713 to 2732, 2714 to 2733, 2715 to 2734, 2716 to 2735, 2717 to 2736, 2718 to 2737, 2719 to 2738, 2720 to 2739, 2721 to 2740, 2722 to 2741, 2723 to 2742, 2724 to 2743, 2725 to 2744, 2726 to 2745, 2727 to 2746, 2728 to 2747, 2729 to 2748, 2730 to 2749, 2731 to 2750, 2732 to 2751, 2733 to 2752, 2775 to 2794, 2790 to 2809, 2826 to 2845, 2827 to 2846, 2828 to 2847, 2829 to 2848, 2830 to 2849, 2831 to 2850, 2832 to 2851, 2833 to 2852, 2834 to 2853, 2835 to 2854, 2836 to 2855, 2837 to 2856, 2838 to 2857, 2839 to 2858, 2840 to 2859, 2841 to 2860, 2842 to 2861, 2843 to 2862, 2844 to 2863, 2962 to 2981, 2963 to 2982, 3032 to 3051, 3052 to 3071, 3168 to 3187, 3232 to 3251, 3251 to 3270, 3302 to 3321, 3303 to 3322, 3304 to 3323, 3305 to 3324, 3306 to 3325, 3307 to 3326, 3308 to 3327, 3309 to 3328, 3310 to 3329, 3311 to 3330, 3312 to 3331, 3313 to 3332, 3314 to 3333, 3315 to 3334, 3316 to 3335, 3317 to 3336, 3318 to 3337, 3319 to 3338, 3320 to 3339, 3321 to 3340, 3322 to 3341, 3323 to 3342, 3324 to 3343, 3408 to 3427, 3590 to 3609, 3608 to 3627, 3617 to 3636, 3618 to 3637, 3619 to 3638, 3620 to 3639, 3621 to 3640, 3622 to 3641, 3623 to 3642, 3624 to 3643, 3625 to 3644, 3626 to 3645, 3627 to 3646, 3628 to 3647, 3629 to 3648, 3630 to 3649, 3631 to 3650, 3632 to 3651, 3633 to 3652, 3634 to 3653, 3635 to 3654, 3636 to 3655, 3637 to 3656, 3638 to 3657, 3639 to 3658, 3640 to 3659, 3641 to 3660, 3642 to 3661, 3643 to 3662, 3644 to 3663, 3645 to 3664, 3646 to 3665, 3647 to 3666, 3648 to 3667, 3649 to 3668, 3650 to 3669, 3651 to 3670, 3652 to 3671, 3653 to 3672, 3654 to 3673, 3671 to 3690, 3672 to 3691, 3673 to 3692, 3674 to 3693, 3675 to 3694, 3676 to 3695, 3677 to 3696, 3678 to 3697, 3679 to 3698, 3680 to 3699, 3681 to 3700, 3682 to 3701, 3683 to 3702, 3684 to 3703, 3685 to 3704, 3686 to 3705, 3687 to 3706, 3688 to 3707, 3689 to 3708, 3690 to 3709, 3691 to 3710, 3692 to 3711, 3693 to 3712, 3694 to 3713, 3695 to 3714, 3696 to 3715, 3697 to 3716, 3698 to 3717, 3699 to 3718, 3700 to 3719, 3701 to 3720, 3702 to 3721, 3703 to 3722, 3704 to 3723, 3705 to 3724, 3706 to 3725, 3707 to 3726, 3708 to 3727, 3709 to 3728, 3739 to 3758, 3740 to 3759, 3741 to 3760, 3742 to 3761, 3743 to 3762, 3744 to 3763, 3745 to 3764, 3877 to 3896, 3949 to 3968, 3964 to 3983, 3965 to 3984, 3966 to 3985, 3967 to 3986, 3968 to 3987, 3969 to 3988, 3970 to 3989, 3971 to 3990, 3972 to 3991, 3973 to 3992, 3974 to 3993, 3975 to 3994, 3976 to 3995, 3977 to 3996, 3978 to 3997, 3979 to 3998, 3980 to 3999, 3981 to 4000, 3982 to 4001, 3983 to 4002, 3984 to 4003, 3985 to 4004, 3986 to 4005, 3987 to 4006, 3988 to 4007, 3989 to 4008, 3990 to 4009, 3991 to 4010, 3992 to 4011, 3993 to 4012, 3994 to 4013, 3995 to 4014, 3996 to 4015, 3997 to 4016, 3998 to 4017, 3999 to 4018, 4011 to 4030, 4177 to 4196, 4178 to 4197, 4179 to 4198, 4180 to 4199, 4181 to 4200, 4182 to 4201, 4183 to 4202, 4184 to 4203, 4185 to 4204, 4186 to 4205, 4187 to 4206, 4188 to 4207, 4189 to 4208, 4190 to 4209, 4191 to 4210, 4192 to 4211, 4193 to 4212, 4194 to 4213, 4195 to 4214, 4196 to 4215, 4197 to 4216, 4198 to 4217, 4199 to 4218, 4200 to 4219, 4201 to 4220, 4202 to 4221, 4203 to 4222, 4204 to 4223, 4205 to 4224, and 4206 to 4225 of the base sequence of SEQ ID NO: 471.

7. The antisense oligonucleotide or a pharmaceutically acceptable salt thereof, or a hydrate thereof according to claim 3, wherein the antisense oligonucleotide is complementary to a nucleic acid comprising at least 15 consecutive bases in a target region selected from the group consisting of positions 14 to 33, 34 to 53, 54 to 73, 148 to 167, 397 to 432, 747 to 778, 965 to 984, 1127 to 1146, 1148 to 1173, 1182 to 1213, 1228 to 1254, 1270 to 1308, 1484 to 1564, 1578 to 1629, 1686 to 1705, 1748 to 1767, 1768 to 1787, 1794 to 1813, 1870 to 1913, 2015 to 2034, 2086 to 2118, 2129 to 2148, 2151 to 2170, 2206 to 2225, 2239 to 2272, 2287 to 2336, 2337 to 2373, 2387 to 2406, 2598 to 2632, 2633 to 2668, 2672 to 2691, 2699 to 2757, 2775 to 2809, 2826 to 2866, 2959 to 2978, 3032 to 3051, 3052 to 3071, 3136 to 3155, 3168 to 3187, 3188 to 3207, 3232 to 3270, 3298 to 3317, 3325 to 3344, 3408 to 3427, 3590 to 3690, 3691 to 3728, 3739 to 3769, 3877 to 3896, 3949 to 3983, 3984 to 4030, and 4170 to 4198 of the base sequence of SEQ ID NO: 471.

8. The antisense oligonucleotide or a pharmaceutically acceptable salt thereof, or a hydrate thereof according to claim 7, wherein the antisense oligonucleotide is complementary to a nucleic acid comprising at least 15 consecutive bases in a target region selected from the group consisting of positions 14 to 33, 34 to 53, 54 to 73, 148 to 167, 397 to 416, 413 to 432, 747 to 766, 756 to 775, 759 to 778, 965 to 984, 1127 to 1146, 1148 to 1167, 1151 to 1170, 1154 to 1173, 1182 to 1201, 1185 to 1204, 1188 to 1207, 1191 to 1210, 1194 to 1213, 1228 to 1247, 1231 to 1250, 1235 to 1254, 1270 to 1289, 1289 to 1308, 1484 to 1503, 1488 to 1507, 1491 to 1510, 1493 to 1512, 1506 to 1525, 1517 to 1536, 1531 to 1550, 1545 to 1564, 1578 to 1597, 1586 to 1605, 1589 to 1608, 1592 to 1611, 1595 to 1614, 1598 to 1617, 1601 to 1620, 1604 to 1623, 1607 to 1626, 1610 to 1629, 1686 to 1705, 1748 to 1767, 1768 to 1787, 1794 to 1813, 1870 to 1889, 1871 to 1890, 1874 to 1893, 1877 to 1896, 1880 to 1899, 1883 to 1902, 1886 to 1905, 1889 to 1908, 1894 to 1913, 2015 to 2034, 2086 to 2105, 2097 to 2116, 2099 to 2118, 2129 to 2148, 2151 to 2170, 2206 to 2225, 2239 to 2258, 2253 to 2272, 2287 to 2306, 2301 to 2320, 2317 to 2336, 2337 to 2356, 2354 to 2373, 2387 to 2406, 2598 to 2617, 2613 to 2632, 2633 to 2652, 2649 to 2668, 2672 to 2691, 2699 to 2718, 2702 to 2721, 2705 to 2724, 2708 to 2727, 2711 to 2730, 2714 to 2733, 2717 to 2736, 2720 to 2739, 2723 to 2742, 2726 to 2745, 2729 to 2748, 2738 to 2757, 2775 to 2794, 2790 to 2809, 2826 to 2845, 2829 to 2848, 2832 to 2851, 2835 to 2854, 2838 to 2857, 2841 to 2860, 2847 to 2866, 2959 to 2978, 3032 to 3051, 3052 to 3071, 3136 to 3155, 3168 to 3187, 3188 to 3207, 3232 to 3251, 3251 to 3270, 3298 to 3317, 3325 to 3344, 3408 to 3427, 3590 to 3609, 3608 to 3627, 3617 to 3636, 3635 to 3654, 3654 to 3673, 3671 to 3690, 3691 to 3710, 3709 to 3728, 3739 to 3758, 3750 to 3769, 3877 to 3896, 3949 to 3968, 3964 to 3983, 3984 to 4003, 3999 to 4018, 4011 to 4030, 4170 to 4189, 4173 to 4192, 4176 to 4195, and 4179 to 4198 of the base sequence of SEQ ID NO: 471.

9. The antisense oligonucleotide or a pharmaceutically acceptable salt thereof, or a hydrate thereof according to claim 5 or 7, wherein the antisense oligonucleotide is complementary to a nucleic acid comprising at least 15 consecutive bases in a target region selected from the group consisting of positions 14 to 33, 34 to 53, 747 to 766, 1148 to 1167, 1289 to 1308, 1484 to 1564, 1578 to 1623, 1686 to 1705, 1748 to 1767, 1768 to 1787, 1794 to 1813, 1877 to 1905, 2151 to 2170, 2206 to 2225, 2239 to 2272, 2287 to 2336, 2337 to 2373, 2387 to 2406, 2613 to 2632, 2633 to 2668, 2672 to 2691, 2699 to 2748, 2775 to 2809, 2826 to 2860, 3032 to 3051, 3052 to 3071, 3168 to 3187, 3232 to 3270, 3408 to 3427, 3590 to 3690, 3691 to 3728, 3739 to 3758, 3877 to 3896, 3949 to 3983, 3984 to 4030, and 4179 to 4198 of the base sequence of SEQ ID NO: 471.

10. The antisense oligonucleotide or a pharmaceutically acceptable salt thereof, or a hydrate thereof according to claim 9, wherein the antisense oligonucleotide is complementary to a nucleic acid comprising at least 15 consecutive bases in a target region selected from the group consisting of positions 14 to 33, 34 to 53, 747 to 766, 1148 to 1167, 1289 to 1308, 1484 to 1503, 1488 to 1507, 1491 to 1510, 1493 to 1512, 1506 to 1525, 1517 to 1536, 1531 to 1550, 1545 to 1564, 1578 to 1597, 1586 to 1605, 1589 to 1608, 1592 to 1611, 1595 to 1614, 1598 to 1617, 1601 to 1620, 1604 to 1623, 1686 to 1705, 1748 to 1767, 1768 to 1787, 1794 to 1813, 1877 to 1896, 1880 to 1899, 1883 to 1902, 1886 to 1905, 2151 to 2170, 2206 to 2225, 2239 to 2258, 2253 to 2272, 2287 to 2306, 2301 to 2320, 2317 to 2336, 2337 to 2356, 2354 to 2373, 2387 to 2406, 2613 to 2632, 2633 to 2652, 2649 to 2668, 2672 to 2691, 2699 to 2718, 2702 to 2721, 2705 to 2724, 2708 to 2727, 2711 to 2730, 2714 to 2733, 2717 to 2736, 2720 to 2739, 2723 to 2742, 2726 to 2745, 2729 to 2748, 2775 to 2794, 2790 to 2809, 2826 to 2845, 2829 to 2848, 2832 to 2851, 2835 to 2854, 2838 to 2857, 2841 to 2860, 3032 to 3051, 3052 to 3071, 3168 to 3187, 3232 to 3251, 3251 to 3270, 3408 to 3427, 3590 to 3609, 3608 to 3627, 3617 to 3636, 3635 to 3654, 3654 to 3673, 3671 to 3690, 3691 to 3710, 3709 to 3728, 3739 to 3758, 3877 to 3896, 3949 to 3968, 3964 to 3983, 3984 to 4003, 3999 to 4018, 4011 to 4030, and 4179 to 4198 of the base sequence of SEQ ID NO: 471.

11. The antisense oligonucleotide or a pharmaceutically acceptable salt thereof, or a hydrate thereof according to claim 7, wherein the antisense oligonucleotide is complementary to a nucleic acid comprising at least 15 consecutive bases in a target region selected from the group consisting of positions 1598 to 1626, 1870 to 1890, 2097 to 2116, 2711 to 2736, 2841 to 2860, and 4170 to 4198 of the base sequence of SEQ ID NO: 471.

12. The antisense oligonucleotide or a pharmaceutically acceptable salt thereof, or a hydrate thereof according to claim 7 or 8, wherein the antisense oligonucleotide is complementary to a nucleic acid comprising at least 15 consecutive bases in a target region selected from the group consisting of positions 1598 to 1617, 1604 to 1623, 1607 to 1626, 1870 to 1889, 1871 to 1890, 2097 to 2116, 2711 to 2730, 2714 to 2733, 2717 to 2736, 2841 to 2860, 4170 to 4189, 4173 to 4192, and 4179 to 4198 of the base sequence of SEQ ID NO: 471.

13. The antisense oligonucleotide or a pharmaceutically acceptable salt thereof, or a hydrate thereof according to any one of claims 1 to 12, wherein the antisense oligonucleotide is complementary to a nucleic acid comprising at least 20 consecutive bases in the target region.

14. The antisense oligonucleotide or a pharmaceutically acceptable salt thereof, or a hydrate thereof according to claim 3 or 4, comprising:
(i) a base sequence selected from the group consisting of SEQ ID NOs: 1 to 6, 8, 9, 11, 14 to 24, 26, 29, 31 to 60, 66 to 67, 73 to 75, 79 to 82, 86 to 89, 92 to 111, 115 to 136, 138 to 145, 147 to 158, and 160 to 464;
(ii) a base sequence having addition, deletion, or substitution of one or several base(s) in a base sequence selected from the group consisting of SEQ ID NOs: 1 to 6, 8, 9, 11, 14 to 24, 26, 29, 31 to 60, 66 to 67, 73 to 75, 79 to 82, 86 to 89, 92 to 111, 115 to 136, 138 to 145, 147 to 158, and 160 to 464; or
(iii) a base sequence having 90% or more sequence identity with a base sequence selected from the group consisting of SEQ ID NOs: 1 to 6, 8, 9, 11, 14 to 24, 26, 29, 31 to 60, 66 to 67, 73 to 75, 79 to 82, 86 to 89, 92 to 111, 115 to 136, 138 to 145, 147 to 158, and 160 to 464.

15. The antisense oligonucleotide or a pharmaceutically acceptable salt thereof, or a hydrate thereof according to claim 5 or 6, comprising:
(i) a base sequence selected from the group consisting of SEQ ID NOs: 1, 5, 6, 14 to 24, 31 to 35, 37 to 40, 42 to 45, 47 to 50, 52 to 59, 73, 92 to 100, 103, 107 to 110, 120 to 136, 138 to 143, 150 to 153, 155 to 158, and 163 to 464;
(ii) a base sequence having addition, deletion, or substitution of one or several base(s) in a base sequence selected from the group consisting of SEQ ID NOs: 1, 5, 6, 14 to 24, 31 to 35, 37 to 40, 42 to 45, 47 to 50, 52 to 59, 73, 92 to 100, 103, 107 to 110, 120 to 136, 138 to 143, 150 to 153, 155 to 158, and 163 to 464; or
(iii) a base sequence having 90% or more sequence identity with a base sequence selected from the group consisting of SEQ ID NOs: 1, 5, 6, 14 to 24, 31 to 35, 37 to 40, 42 to 45, 47 to 50, 52 to 59, 73, 92 to 100, 103, 107 to 110, 120 to 136, 138 to 143, 150 to 153, 155 to 158, and 163 to 464.

16. The antisense oligonucleotide or a pharmaceutically acceptable salt thereof, or a hydrate thereof according to claim 7 or 8, comprising:
(i) a base sequence selected from the group consisting of SEQ ID NOs: 1 to 4, 6, 8, 9, 11, 14 to 24, 26, 29, 31 to 37, 39 to 50, 52 to 60, 66 to 67, 73 to 75, 79 to 82, 86 to 89, 92 to 111, 115 to 118, 120 to 136, 138 to 145, 147 to 158, and 160 to 163;
(ii) a base sequence having addition, deletion, or substitution of one or several base(s) in a base sequence selected from the group consisting of SEQ ID NOs: 1 to 4, 6, 8, 9, 11, 14 to 24, 26, 29, 31 to 37, 39 to 50, 52 to 60, 66 to 67, 73 to 75, 79 to 82, 86 to 89, 92 to 111, 115 to 118, 120 to 136, 138 to 145, 147 to 158, and 160 to 163; or
(iii) a base sequence having 90% or more sequence identity with a base sequence selected from the group consisting of SEQ ID NOs: 1 to 4, 6, 8, 9, 11, 14 to 24, 26, 29, 31 to 37, 39 to 50, 52 to 60, 66 to 67, 73 to 75, 79 to 82, 86 to 89, 92 to 111, 115 to 118, 120 to 136, 138 to 145, 147 to 158, and 160 to 163.

17. The antisense oligonucleotide or a pharmaceutically acceptable salt thereof, or a hydrate thereof according to claim 9 or 10, comprising:
(i) a base sequence selected from the group consisting of SEQ ID NOs: 1, 6, 14 to 24, 31 to 35, 37, 39, 40, 42 to 45, 47 to 50, 52 to 59, 73, 92 to 100, 103, 107 to 110, 120 to 136, 138 to 143, 150 to 153, 155 to 158, and 163;
(ii) a base sequence having addition, deletion, or substitution of one or several base(s) in a base sequence selected from the group consisting of SEQ ID NOs: 1, 6, 14 to 24, 31 to 35, 37, 39, 40, 42 to 45, 47 to 50, 52 to 59, 73, 92 to 100, 103, 107 to 110, 120 to 136, 138 to 143, 150 to 153, 155 to 158, and 163; or
(iii) a base sequence having 90% or more sequence identity with a base sequence selected from the group consisting of SEQ ID NOs: 1, 6, 14 to 24, 31 to 35, 37, 39, 40, 42 to 45, 47 to 50, 52 to 59, 73, 92 to 100, 103, 107 to 110, 120 to 136, 138 to 143, 150 to 153, 155 to 158, and 163.

18. The antisense oligonucleotide or a pharmaceutically acceptable salt thereof, or a hydrate thereof according to claim 11 or 12, comprising:
(i) a base sequence selected from the group consisting of SEQ ID NOs: 98, 100, 101, 104, 105, 116, 131 to 133, 143, 160, 161, and 163;
(ii) a base sequence having addition, deletion, or substitution of one or several base(s) in a base sequence selected from the group consisting of SEQ ID NOs: 98, 100, 101, 104, 105, 116, 131 to 133, 143, 160, 161, and 163; or
(iii) a base sequence having 90% or more sequence identity with a base sequence selected from the group consisting of SEQ ID NOs: 98, 100, 101, 104, 105, 116, 131 to 133, 143, 160, 161, and 163.

19. The antisense oligonucleotide or a pharmaceutically acceptable salt thereof, or a hydrate thereof according to claim 11 or 12, comprising:
(i) a base sequence selected from the group consisting of SEQ ID NOs: 21, 164 to 222, 233, 234, and 350 to 355;
(ii) a base sequence having addition, deletion, or substitution of one or several base(s) in a base sequence selected from the group consisting of SEQ ID NOs: 21, 164 to 222, 233, 234, and 350 to 355; or
(iii) a base sequence having 90% or more sequence identity with a base sequence selected from the group consisting of SEQ ID NOs: 21, 164 to 222, 233, 234, and 350 to 355.

20. The antisense oligonucleotide or a pharmaceutically acceptable salt thereof, or a hydrate thereof according to claim 11 or 12, comprising:
(i) a base sequence selected from the group consisting of SEQ ID NOs: 170, 177, 196, 199, and 221;
(ii) a base sequence having addition, deletion, or substitution of one or several base(s) in a base sequence selected from the group consisting of SEQ ID NOs: 170, 177, 196, 199, and 221; or
(iii) a base sequence having 90% or more sequence identity with a base sequence selected from the group consisting of SEQ ID NOs: 170, 177, 196, 199, and 221.

21. The antisense oligonucleotide or a pharmaceutically acceptable salt thereof, or a hydrate thereof according to any one of claims 14 to 20, comprising the base sequence (i).

22. An antisense oligonucleotide consisting of 15 to 22 nucleotides complementary to a nucleic acid comprising at least 15 consecutive bases in a target region selected from the group consisting of positions 468 to 487, 474 to 493, 583 to 602, 586 to 605, 619 to 638, 770 to 789, 774 to 793, 775 to 794, 778 to 797, 804 to 823, 851 to 870, 1160 to 1179, 1162 to 1181, 1170 to 1189, 1173 to 1192, 1205 to 1224, 1210 to 1229, 1216 to 1235, 1217 to 1236, 1219 to 1238, 1385 to 1404, 1441 to 1460, 1818 to 1837, 1902 to 1921, 1905 to 1924, 1908 to 1927, 1914 to 1933, 1931 to 1950, 2117 to 2136, 2749 to 2768, 3100 to 3119, and 4125 to 4144 of a base sequence of SEQ ID NO: 471, or a pharmaceutically acceptable salt thereof, or a hydrate thereof.

23. The antisense oligonucleotide or a pharmaceutically acceptable salt thereof, or a hydrate thereof according to claim 22, comprising:
(i) a base sequence selected from the group consisting of SEQ ID NOs: 7, 10, 12, 13, 25, 27, 28, 30, 61 to 65, 68 to 72, 76 to 78, 83 to 85, 90, 91, 112 to 114, 137, 146, and 159;
(ii) a base sequence having addition, deletion, or substitution of one or several base(s) in a base sequence selected from the group consisting of SEQ ID NOs: 7, 10, 12, 13, 25, 27, 28, 30, 61 to 65, 68 to 72, 76 to 78, 83 to 85, 90, 91, 112 to 114, 137, 146, and 159; or
(iii) a base sequence having 90% or more sequence identity with a base sequence selected from the group consisting of SEQ ID NOs: 7, 10, 12, 13, 25, 27, 28, 30, 61 to 65, 68 to 72, 76 to 78, 83 to 85, 90, 91, 112 to 114, 137, 146, and 159.

24. The antisense oligonucleotide or a pharmaceutically acceptable salt thereof, or a hydrate thereof according to claim 23, comprising the base sequence (i).

25. The antisense oligonucleotide or a pharmaceutically acceptable salt thereof, or a hydrate thereof according to any one of claims 1 to 24, wherein the antisense oligonucleotide consists of 20 nucleotides.

26. The antisense oligonucleotide or a pharmaceutically acceptable salt thereof, or a hydrate thereof according to any one of claims 1 to 25, wherein a sugar moiety and/or a phosphate-binding region of at least one nucleotide constituting the oligonucleotide is modified.

27. The antisense oligonucleotide or a pharmaceutically acceptable salt thereof, or a hydrate thereof according to claim 26, wherein the antisense oligonucleotide is a gapmer comprising a gap region at the center, and two wing regions adjacent to the gap region on the 5′ end side and the 3′ end side (a 5′ wing region and a 3′ wing region).

28. The antisense oligonucleotide or a pharmaceutically acceptable salt thereof, or a hydrate thereof according to claim 27, wherein bonds between nucleosides are all phosphorothioate bonds.

29. The antisense oligonucleotide or a pharmaceutically acceptable salt thereof, or a hydrate thereof according to claim 27, wherein one or more of a bond between the 2nd and 3rd nucleosides, a bond between the 3rd and 4th nucleosides, and a bond between the 4th and 5th nucleosides from the 5′ side of the 5′ wing region are phosphodiester bonds, and/or one or more of a bond between the 1st and 2nd nucleosides, a bond between the 2nd and 3rd nucleosides, and a bond between the 3rd and 4th nucleosides from the 5′ side of the 3′ wing region are phosphodiester bonds, and bonds between the other nucleosides are all phosphorothioate bonds.

30. The antisense oligonucleotide or a pharmaceutically acceptable salt thereof, or a hydrate thereof according to claim 29, wherein the bond between the 2nd and 3rd nucleosides, and the bond between the 4th and 5th nucleosides from the 5′ side of the 5′ wing region are phosphodiester bonds, and/or the bond between the 1st and 2nd nucleosides, and the bond between the 3rd and 4th nucleosides from the 5′ side of the 3′ wing region are phosphodiester bonds, and bonds between the other nucleosides are all phosphorothioate bonds.

31. The antisense oligonucleotide or a pharmaceutically acceptable salt thereof, or a hydrate thereof according to claim 29, wherein the bond between the 2nd and 3rd nucleosides, the bond between the 3rd and 4th nucleosides, and the bond between the 4th and 5th nucleosides from the 5′ side of the 5′ wing region are phosphodiester bonds, and/or the bond between the 1st and 2nd nucleosides, the bond between the 2nd and 3rd nucleosides, and the bond between the 3rd and 4th nucleosides from the 5′ side of the 3′ wing region are phosphodiester bonds, and bonds between the other nucleosides are all phosphorothioate bonds.

32. The antisense oligonucleotide or a pharmaceutically acceptable salt thereof, or a hydrate thereof according to any one of claims 27 to 31, wherein the wing region comprises a 2′-OMe (2′-O—CH₃) group and/or a 2′-O-MOE (2′-O—CH₂CH₂OCH₃) group.

33. A pharmaceutical composition comprising the antisense oligonucleotide or a pharmaceutically acceptable salt thereof, or a hydrate thereof according to any one of claims 1 to 32.

34. The antisense oligonucleotide or a pharmaceutically acceptable salt thereof, or a hydrate thereof according to any one of claims 1 to 32, or the pharmaceutical composition according to claim 33, for treating and/or preventing dentatorubral-pallidoluysian atrophy (DRPLA).

35. A method for treating and/or preventing dentatorubral-pallidoluysian atrophy (DRPLA), comprising administering, to a subject, the antisense oligonucleotide or a pharmaceutically acceptable salt thereof, or a hydrate thereof according to any one of claims 1 to 32, or the pharmaceutical composition according to claim 33 or 34.
